(19) United States
(12) Patent Application Publication
(10) Pub. No.: US 2014/0357557 A1
(43) Pub. Date: Dec. 4, 2014

(54) CYCLODEXTRIN-BASED POLYMERS FOR THERAPEUTIC DELIVERY

(71) Applicants: Roderic O. Cole, Plaistow, NH (US); Derek Gregory van der Poll, Somerville, MA (US)

(72) Inventors: Roderic O. Cole, Plaistow, NH (US); Derek Gregory van der Poll, Somerville, MA (US)

(73) Assignee: CERULEAN PHARMA INC., Cambridge, MA (US)

(21) Appl. No.: 14/291,745

(22) Filed: May 30, 2014

Related U.S. Application Data
(60) Provisional application No. 61/829,797, filed on May 31, 2013.

(51) Int. Cl. A61K 47/48; A61K 31/519; A61K 45/06; A61K 38/05

(52) U.S. Cl. CPC A61K 47/4823; A61K 38/05; A61K 31/519; A61K 45/06
USPC 514/7.3; 536/17.4; 514/19.3; 514/16.6; 514/6.9; 514/16.8

(57) ABSTRACT

Methods and compositions relating to CDP-JAK inhibitor conjugates are described herein.

This application claims priority to U.S. Ser. No. 61/829,797 filed May 31, 2013, the entire contents of which is incorporated herein by reference.

### BACKGROUND

Drug delivery of some small molecule therapeutic agents has been problematic due to their poor pharmacological profiles. These therapeutic agents often have low aqueous solubility, their bioactive forms exist in equilibrium with an inactive form, or high systemic concentrations of the agents lead to toxic side-effects.

### SUMMARY

In one aspect, the disclosure features a CDP-janus kinase (JAK) inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, and methods of making the CDP-JAK inhibitor conjugates, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate.

In one embodiment, CDP is not biodegradable.

In one embodiment, CDP is biocompatible.

In one embodiment, the CDP-CDP-JAK inhibitor conjugate, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, includes an inclusion complex between a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), attached or conjugated to the CDP, e.g., via a covalent linkage or via a linker such as a linker described herein, and another molecule in the CDP. In one embodiment, the CDP-JAK inhibitor conjugate forms a nanoparticle. In one embodiment, the CDP-JAK inhibitor conjugate including an inclusion complex forms a nanoparticle. The nanoparticle ranges in size from 10 to 300 nm in diameter, e.g., 10 to 280, 20 to 280, 30 to 250, 30 to 200, 20 to 150, 30 to 100, 20 to 80, 10 to 80, 10 to 70, 20 to 60 or 20 to 50 nm 10 to 70, 10 to 60 or 10 to 50 nm diameter. In one embodiment, the nanoparticle is 20 to 60 nm in diameter. In one embodiment, the composition comprises a population or a plurality of nanoparticles with an average diameter from 10 to 300 nm, e.g., 20 to 280, 15 to 250, 15 to 200, 20 to 150, 15 to 100, 20 to 80, 15 to 80, 15 to 70, 15 to 60, 15 to 50, or 20 to 50 nm. In one embodiment, the average nanoparticle diameter is from 15 to 60 nm (e.g., 20-60). In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

In an embodiment, the CDP-JAK inhibitor conjugate complex forms a particle or nanoparticle having a conjugate number described herein. By way of example, a CDP-JAK inhibitor conjugate described herein, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-JAK inhibitor conjugate described herein is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one embodiment, the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), conjugated to the CDP is more soluble when conjugated to the CDP, than when not conjugated to the CDP.

In one embodiment, the composition comprises a population, mixture or plurality of CDP-JAK inhibitor conjugates. In one embodiment, the population, mixture or plurality of CDP-JAK inhibitor conjugates comprises a plurality of different JAK inhibitors conjugated to a CDP (e.g., two different JAK inhibitors are in the composition such that two different JAK inhibitors are attached to a single CDP; or a first JAK inhibitor is attached to a first CDP and a second JAK inhibitor is attached to a second CDP and both CDP-JAK inhibitor conjugates are present in the composition). In one embodiment, the population, mixture or plurality of CDP-JAK inhibitor conjugates comprises a CDP having a single JAK inhibitor attached thereto in a plurality of positions (e.g., a CDP has a single JAK inhibitor attached thereto such that the single JAK inhibitor for some occurrences is attached to the CDP through the N-5 of the JAK inhibitor, e.g., pyrrolopyrimidine of ruxolitinib, baricitinib, tofacitinib, GLPG0634).

In some embodiments, the JAK inhibitor is attached to the CDP through a hydroxyl group, e.g., a primary or secondary hydroxyl group. In some embodiment, the JAK inhibitor is attached to the CDP through the primary hydroxyl group of a JAK inhibitor, e.g., lestaurtinib. In some embodiment, the JAK inhibitor is attached to the CDP through the secondary hydroxyl group of a JAK inhibitor, e.g., lestaurtinib.

In some embodiments, the JAK inhibitor is attached to the CDP through a nitrogen atom on the JAK inhibitor, e.g., a primary or secondary nitrogen. In some embodiments, the JAK inhibitor is attached to the CDP through the pyrrole nitrogen of a pyrrolopyrimidine moiety on the JAK inhibitor, e.g., ruxolitinib, baricitinib, tofacitinib, or GLPG0634.

In some embodiments, the JAK inhibitor is attached to the CDP through an aniline nitrogen on the JAK inhibitor. In some embodiments, the JAK inhibitor is attached to the CDP through the imidazole pyrazole nitrogen on the JAK inhibitor. In some embodiments, the JAK inhibitor is attached to the CDP through a secondary nitrogen of the JAK inhibitor, e.g., through the azepine and/or the imidazole nitrogen of INCB16562. In some embodiments, the JAK inhibitor is attached to the CDP through one or both of the secondary nitrogens pyrimidinoamine, and/or the pyrazole nitrogen of AZD1480. In some embodiments, the JAK inhibitor is attached to the CDP through the aniline nitrogen and/or the pyrrolidine nitrogen on the JAK inhibitor, e.g., XL019. In some embodiments, the JAK inhibitor is attached to the CDP through the aniline nitrogen on the JAK inhibitor, e.g., pacritinib, CYT387, CEP33779, and TG101348. In some embodiments, the JAK inhibitor is attached to the CDP through the piperidinyl nitrogen to the JAK inhibitor, e.g., NVP-BSK805.

In an embodiment, the CDP-JAK inhibitor conjugate comprises a JAK inhibitor, e.g., a JAK inhibitor described herein, e.g., a JAK1, JAK2, JAK3 and/or Tyk2 inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein. In an embodiment, the CDP-JAK inhibitor conjugate comprises a JAK inhibitor, coupled via a linker as disclosed herein to a CDP moiety, e.g., a CDP described herein. In an embodiment, the CDP-JAK inhibitor conjugate is a CDP-JAK inhibitor conjugate disclosed herein and in FIGS. 1-11.

In one aspect, the disclosure features a method of treating a disorder, e.g., an inflammatory disorder, an autoimmune disorder, or a proliferative disorder, e.g., a cancer, in a subject, e.g., a human, the method comprises: administering a composition that comprises a CDP-JAK inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the disorder, e.g., the inflammatory disorder, the autoimmune disorder, or the proliferative disorder, e.g., cancer, to thereby treat the disorder, e.g., the inflammatory disorder, the autoimmune disorder, or the proliferative disorder, e.g., cancer.

In one embodiment, the CDP-JAK inhibitor conjugate, e.g., the CDP-JAK inhibitor conjugate described herein, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate) is administered by subcutaneous administration. In one embodiment, the CDP-JAK inhibitor conjugate, e.g., the CDP-JAK inhibitor conjugate described herein, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate) is administered by intravenous administration.

In one embodiment, the CDP-JAK inhibitor, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), is administered by subcutaneous administration, and one or more subsequent doses of the CDP-JAK inhibitor, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate) is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), is administered at a dose of 1 mg/kg to 25 mg/kg (e.g., 1 mg/kg, 2 mg/kg, 3 mg/kg, 4 mg/kg, 5 mg/kg for a CDP-tofacitinib conjugate, or e.g., 1 mg/kg, 5 mg/kg, 10 mg/kg, 15 mg/kg, 20 mg/kg, 25 mg/kg for a CDP-ruxolitinib conjugate) wherein the dosage is expressed in mg of drug, as opposed to mg of conjugate. In one embodiment, the method further comprises administering one or more subsequent doses of the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), e.g., at a dose of 1 mg/kg to 5 mg/kg for a CDP-tofacitinib conjugate (e.g., 1 mg/kg, 2 mg/kg, 3 mg/kg, 4 mg/kg, 5 mg/kg), or at a dose of 1 mg/kg to 25 mg/kg for a CDP-ruxolitinib conjugate (e.g., 1 mg/kg, 5 mg/kg, 10 mg/kg, 15 mg/kg, 20 mg/kg, 25 mg/kg) wherein the dosage is expressed in mg of drug, as opposed to mg of conjugate. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the disorder. In one embodiment, each subsequent dose is one week, two weeks, three weeks or four weeks after the previous dose.

In one embodiment, the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), is administered at a dose of 0.01 mg/kg to 0.50 mg/kg (e.g., 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg) of tofacitinib (wherein the dosage is expressed in mg of drug, as opposed to mg of conjugate). In one embodiment, the method further comprises administering one or more subsequent doses of the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the disorder. In one embodiment, each subsequent dose is one week, two weeks, three weeks or four weeks after the previous dose.

In one embodiment, the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), is administered at a dose of 0.05 mg/kg to 2 mg/kg (e.g., 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-pyrrolopyrimidine-containing JAK inhibitor conjugate (e.g., the CDP-tofacitinib conjugate, the CDP-ruxolitinib conjugate, the CDP-baricitinib conjugate, the CDP-GLPG0634 conjugate), e.g., at a dose of 0.05 mg/kg to 2 mg/kg (e.g., 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/k. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the disorder. In one embodiment, each subsequent dose is one week, two weeks, three weeks or four weeks after the previous dose.

In one embodiment, the composition includes a CDP-INCB16562 conjugate, e.g., a CDP-INCB16562 conjugate described herein, e.g., a CDP-INCB16562 conjugate comprising INCB16562 molecules, coupled, e.g., via linkers, to a CDP described herein. In one embodiment, the CDP-INCB16562 conjugate is administered at a dose and/or dosing schedule described herein.

In one embodiment, the composition includes a CDP-XL019 conjugate, e.g., a CDP-XL019 conjugate described herein, e.g., a CDP-XL019 conjugate comprising lestaurtinib molecules, coupled, e.g., via linkers, to a CDP described herein. In one embodiment, the CDP-XL019 conjugate is administered at a dose and/or dosing schedule described herein.

In one embodiment, the composition includes a CDP-pacritinib conjugate, e.g., a CDP-pacritinib conjugate described herein, e.g., a CDP-pacritinib conjugate comprising pacritinib molecules, coupled, e.g., via linkers, to a CDP described herein. In one embodiment, the CDP-pacritinib conjugate is administered at a dose and/or dosing schedule described herein.

In one embodiment, the composition includes a CDP-CYT387 conjugate, e.g., a CDP-CYT387 conjugate described herein, e.g., a CDP-CYT387 conjugate comprising CYT387 molecules, coupled, e.g., via linkers, to a CDP described herein. In one embodiment, the CDP-CYT387 conjugate is administered at a dose and/or dosing schedule described herein.

In one embodiment, the composition includes a CDP-AZD1480 conjugate, e.g., a CDP-AZD1480 conjugate described herein, e.g., a CDP-AZD1480 conjugate comprising AZD1480 molecules, coupled, e.g., via linkers, to a CDP described herein. In one embodiment, the CDP-AZD1480 conjugate is administered at a dose and/or dosing schedule described herein.

In one embodiment, the composition includes a CDP-TG101348 conjugate, e.g., a CDP-TG101348 conjugate described herein, e.g., a CDP-TG101348 conjugate comprising TG101348 molecules, coupled, e.g., via linkers, to a CDP described herein. In one embodiment, the CDP-TG101348 conjugate is administered at a dose and/or dosing schedule described herein.

In one embodiment, the composition includes a CDP-NVP-BSK805 conjugate, e.g., a CDP-NVP-BSK805 conjugate described herein, e.g., a CDP-NVP-BSK805 conjugate comprising NVP-BSK805 molecules, coupled, e.g., via linkers, to a CDP described herein. In one embodiment, the CDP-NVP-BSK805 conjugate is administered at a dose and/or dosing schedule described herein.

In one embodiment, the composition includes a CDP-CEP33779 conjugate, e.g., a CDP-CEP33779 conjugate described herein, e.g., a CDP-CEP33779 conjugate comprising CEP33779 molecules, coupled, e.g., via linkers, to a CDP described herein. In one embodiment, the CDP-CEP33779 conjugate is administered at a dose and/or dosing schedule described herein.

Methods described herein also include the selection of a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia, e.g., a lymphocyte count less than about 500 cells/mm³, neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis). Thus, in one embodiment, the subject, e.g., human subject, is selected on the basis of having or is at risk of developing a malignancy, e.g., other than a successfully treated non-melanoma skin cancer (NMSC), and is administered a CDP-JAK inhibitor conjugate described herein. In another embodiment, the subject, e.g., human subject, is selected on the basis of having or is at risk of developing a renal and/or hepatic impairment, and is administered a CDP-JAK inhibitor conjugate described herein. In another embodiment, the subject, e.g., human subject, is selected on the basis of having or is at risk of developing lymphopenia, e.g., a lymphocyte count less than about 500 cells/mm³, and is administered a CDP-JAK inhibitor conjugate described herein. In another embodiment, the subject, e.g., human subject, is selected on the basis of having or is at risk of developing neutropenia, e.g., an absolute neutrophil count (ANC) of less than 500 cells/mm³, and is administered a CDP-JAK inhibitor conjugate described herein. In another embodiment, the subject, e.g., human subject, is selected on the basis of having or is at risk of developing anemia, e.g., a greater than 2 g/dL decrease or less than 8.0 g/dL in hemoglobin levels, and is administered a CDP-JAK inhibitor conjugate described herein. In another embodiment, the subject, e.g., human subject, is selected on the basis of having or is at risk of developing a serious infection, e.g., due to a bacterial, mycobacterial, fungal, or viral infection, and is administered a CDP-JAK inhibitor conjugate described herein.

In another embodiment, the subject, e.g., human subject, is selected on the basis of having or is at risk of developing elevated liver enzymes, and is administered a CDP-JAK inhibitor conjugate described herein. In another embodiment, the subject, e.g., human subject, is selected on the basis of having or is at risk of developing elevated lipid levels, and is administered a CDP-JAK inhibitor conjugate described herein. In another embodiment, the subject, e.g., human subject, is selected on the basis of having or is at risk of developing a gastrointestinal performation, e.g., due to diverticulitis, and is administered a CDP-JAK inhibitor conjugate described herein.

In another embodiment, the subject, e.g., human subject, is selected on the basis of having received a renal transplant and is at an increased risk of developing Epstein-Barr Virus-associated post-transplant lymphoproliferative disorder, and is administered a CDP-JAK inhibitor conjugate described herein.

In one embodiment, the CDP-JAK inhibitor conjugate composition is administered in combination with one or more additional treatment. In one embodiment, the disorder is a proliferative disorder, e.g., a cancer, and the CDP-JAK inhibitor conjugate is administered in combination with an anticancer agent, e.g., chemotherapeutic agent, e.g., a chemotherapeutic agent or combination of chemotherapeutic agents described herein. In one embodiment, the disorder is a proliferative disorder, e.g., a cancer, and the CDP-JAK inhibitor conjugate is administered in combination with a cancer treatment, e.g., radiation.

In an embodiment, the method further comprises administering a chemotherapeutic agent as a free agent, e.g., a therapeutic agent not bound, e.g., not covalently attached to a polymer.

In an embodiment, the JAK inhibitor associated with the CDP and the free agent are the same chemotherapeutic agent. For example, the agent is a JAK inhibitor (e.g., ruxolitinib, baricitinib, conjugate, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543).

In an embodiment, the JAK inhibitor associated with the CDP and the free agent are different chemotherapeutic agents.

In another embodiment, the method further comprises administering a therapeutic agent other than a chemotherapeutic agent as a free agent, e.g. a therapeutic agent that can treat or prevent one or more side effect associated with administration of the JAK inhibitor.

In one embodiment, the disorder is a disorder other than cancer, e.g., an inflammatory disorder or autoimmune disorder, and the CDP-JAK inhibitor is administered in combination with another treatment, e.g., an agent that can treat or prevent a cardiovascular disease, an inflammatory disorder, an autoimmune disorder, a metabolic disorder, a central nervous system disorder, or a neurological deficit.

In one embodiment, the composition is administered in combination with treatment that ameliorates one or more side effect associated with the JAK inhibitor. For example, in one embodiment, the composition is administered in combination with a treatment for a hematologic disorder, e.g., thrombocytopenia, anemia or neutropenia.

In one embodiment, the composition is administered in combination with a platelet transfusion. In one embodiment, the composition is administered in combination with a blood transfusion.

In one embodiment, the composition is administered in combination with a treatment for renal or hepatic impairment. In one embodiment, the composition is administered in combination with a treatment for lymphopenia. In one embodiment, the composition is administered in combination with a treatment for a serious infection, e.g., due to a bacterial, mycobacterial, fungal, or viral infection. In one embodiment, the composition is administered in combination with a treatment for elevated liver enzymes or elevated lipid levels. In one embodiment, the composition is administered in combination with a treatment for a gastrointestinal performation, e.g., due to diverticulitis.

In another aspect, the disclosure features a method of treating a proliferative disorder, e.g., cancer, in a subject, e.g., a human, the method comprises: administering a composition that comprises a CDP-JAK inhibitor conjugate, e.g., a CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the proliferative disorder, e.g., the cancer, to thereby treat the proliferative disorder. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, blood, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), Castleman's disease, leiomyosarcoma, angiosarcoma, and histiocytoma).

In some embodiments, the cancer is breast cancer (e.g., metastatic or locally advanced breast cancer), prostate cancer (e.g., hormone refractory prostate cancer), pancreatic cancer, squamous cell cancer of the head and neck, lymphoma (Hodgkin's lymphoma (e.g., nodular sclerosing Hodgkin lymphoma (NSHL), mixed cellularity Hodgkin lymphoma (MCHL), lymphocyte depleted Hodgkin lymphoma (LDHL), lymphocyte-rich classic Hodgkin lymphoma (LRCHL), nodular lymphocyte predominant Hodgkin lymphoma (NLPHL)), or non-Hodgkin's lymphoma (e.g., a B-cell lymphoma or a T-cell lymphoma) leukemia (acute myelogenous leukemia (AML), chronic myelogenous leukemia (CML), hairy cell leukemia), gliomas, myeloma (e.g., multiple myeloma), skin cancers such as melanoma (e.g., advanced or metastatic melanoma) and germ cell tumors. Exemplary B-cell lymphomas include diffuse large B-cell lymphoma (e.g., primary mediastinal B-cell lymphoma or intravascular large B-cell lymphoma), follicular lymphoma, small lymphocytic lymphoma, mantle cell lymphoma, marginal zone B-cell lymphoma (e.g., mucosa-associated lymphoid tissue (MALT) lymphoma, nodal marginal zone B-cell lymphoma, splenic marginal zone B-cell lymphoma), Burkitt lymphoma, lymphoplasmacytic lymphoma (Waldenstrom macroglobulinemia), primary central nervous system (CNS) lymphoma. Exemplary T-cell lymphomas include precursor T-lymphoblastic lymphoma/leukemia, peripheral T-cell lymphomas (e.g., cutaneous T-cell lymphomas (such as mycosis fungoides, Sezary syndrome), angioimmunoblastic T-cell lymphoma, extranodal natural killer/T-cell lymphoma, enteropathy type intestinal T-cell lymphoma, anaplastic large cell lymphoma (ALCL), unspecified peripheral T-cell lymphoma).

In another embodiment, the cancer is a cancer of the bone marrow, e.g., a myelofibrosis, e.g., an intermediate or high-risk myelofibrosis, e.g., primary myelofibrosis, post-polycythemia vera myelofibrosis, or post-essential thrombocythemia myelofibrosis.

In another embodiment, the cancer is a cancer known to have a high frequency of mutations in JAK2, e.g., a V617F mutation.

In another embodiment, the cancer is a cancer known to have JAK2 gene fusions, e.g., such as in leukemia patients.

In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid.

In one embodiment, the cancer is resistant to gemcitabine, e.g., gemcitabine resistant pancreatic cancer.

In one embodiment, the proliferative disorder is a myeloproliferative disorder, e.g., polycythemia vera, essential thrombocytosis, myelofibrosis, or myelosclerosis.

In one embodiment, the CDP-JAK inhibitor conjugate, e.g., a CDP-JAK inhibitor conjugate comprising a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via linkers, to a CDP described herein, is administered in combination with one or more additional chemotherapeutic agent. In one embodiment, the CDP-JAK inhibitor conjugate is administered with one or more additional pharmaceutical agents such as, e.g., chemotherapeutics, anti-inflammatory agents, steroids, immunosuppressants, as well as Bcr-Abl, Flt-3, RAF and FAK kinase inhibitors such as, e.g., those described in WO 2006/056399, which is incorporated herein by reference in its entirety.

In some embodiments, the CDP-JAK inhibitor conjugate, e.g., a CDP-JAK inhibitor conjugate comprising a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via linkers, to a CDP described herein, is administered in combination with methotrexate or other nonbiologic disease-modifying antirheumatic drugs (DMARDs).

Example chemotherapeutic agents include: a vinca alkaloid (e.g., vinblastine, vincristine, vindesine and vinorelbine); an alkylating agent (e.g., cyclophosphamide, dacarbazine, melphalan, ifosfamide, temozolomide); a topoisomerase inhibitor (e.g., topotecan, irinotecan, etoposide, teniposide, lamellarin D, SN-38, camptothecin (e.g., CRLX101, formerly known as IT-101)); a platinum-based agent (e.g., cisplatin, carboplatin, oxaliplatin), an antibiotic (e.g., mitomycin, actinomycin, bleomycin), an antimetabolite (e.g., an antifolate, a purine analogue, a pyrimidine analogue (e.g., capecitabine)); an anthracycline (e.g., doxorubicin, daunorubicin, epirubicin, idarubicin, mitoxantrone, valrubicin); a steroid (e.g., prednisone or prednisolone), a taxane (e.g., paclitaxel, docetaxel, larotaxel or cabazitaxel), and proteosome inhibitors (e.g., bortezomib).

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In one embodiment, the disclosure features, a method of treating a subject having a proliferative disorder, e.g., a myeloproliferative disorder, e.g., polycythemia vera, essential thrombocytosis, myelofibrosis, or myelosclerosis, in a subject, e.g., a human subject. The method comprises administering a CDP-JAK inhibitor conjugate, e.g., a CDP-ruxolitinib conjugate, e.g., a CDP-ruxolitinib conjugate described herein, e.g., a CDP-ruxolitinib conjugate comprising ruxolitinib, coupled, e.g., via linkers described herein, to a CDP described herein, to the subject, e.g., human subject, and optionally, providing one or more subsequent administrations of the CDP-JAK inhibitor conjugate, e.g., CDP-ruxolitinib conjugate, e.g., CDP-ruxolitinib conjugate described herein, e.g., CDP-ruxolitinib conjugate comprising ruxolitinib, coupled, e.g., via linkers described herein, to a CDP described herein. In one embodiment, the CDP-ruxolitinib conjugate comprises ruxolitinib coupled via a linker comprising glycine to a CDP described herein. In one embodiment, the CDP-ruxolitinib conjugate comprises ruxolitinib coupled via a linker comprising hexanoate to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

In one embodiment, the CDP-JAK inhibitor, e.g., the CDP-ruxolitinib conjugate, is administered by subcutaneous administration. In one embodiment, the CDP-JAK inhibitor, e.g., the CDP-ruxolitinib conjugate, is administered by intravenous administration.

In another aspect, the disclosure features a method of treating a cardiovascular disease, in a subject, e.g., a human, the method comprises: administering a composition that comprises a CDP-JAK inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the disease, to thereby treat the cardiovascular disease. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis). In one embodiment, the cardiovascular disease is a cardiovascular disease described herein. Examples of cardiovascular diseases include, but are not limited to: angina; arrhythmias (atrial or ventricular or both), or long-standing heart failure; arteriosclerosis; atheroma; atherosclerosis; cardiac hypertrophy including both atrial and ventricular hypertrophy; cardiac or vascular aneurysm; cardiac myocyte dysfunction; carotid obstructive disease; congestive heart failure; endothelial damage after PTCA (percutaneous transluminal coronary angioplasty); hypertension including essential hypertension, pulmonary hypertension and secondary hypertension (renovascular hypertension, chronic glomerulonephritis); myocardial infarction; myocardial ischemia; peripheral obstructive arteriopathy of a limb, an organ, or a tissue; peripheral artery occlusive disease (PAOD); reperfusion injury following ischemia of the brain, heart or other organ or tissue; restenosis; stroke; thrombosis; transient ischemic attack (TIA); vascular occlusion; vasculitis; and vasoconstriction.

In one embodiment, the cardiovascular disease can be an inflammatory disease of the heart such as cardiomyopathy, ischemic heart disease, hypercholesterolemia, and atherosclerosis.

In one embodiment, the CDP-JAK inhibitor is administered in combination with another therapy, e.g., a cardiovascular therapy, e.g., an agent that treats or prevents a cardiovascular disorder.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In another aspect, the disclosure features a method of treating an autoimmune or an inflammatory disease, in a subject, e.g., a human, the method comprises: administering a composition that comprises a CDP-JAK inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the disease, to thereby treat the autoimmune or inflammatory disease. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis). In one embodiment, the autoimmune disease is an autoimmune disease described herein. Examples of autoimmune diseases include, but are not limited to: acute disseminated encephalomyelitis (ADEM); Addison's disease; antiphospholipid antibody syndrome (APS); aplastic anemia; autoimmune hepatitis; cancer; coeliac disease; Crohn's disease; Diabetes mellitus (type 1); Goodpasture's syndrome; Graves' disease; Guillain-Barre syndrome (GBS); Hashimoto's disease; lupus erythematosus; multiple sclerosis; myasthenia gravis; opsoclonus myoclonus syndrome (OMS); optic neuritis; Ord's thyroiditis; oemphigus; polyarthritis; primary biliary cirrhosis; psoriasis; rheumatoid arthritis; Reiter's syndrome; Takayasu's arteritis; temporal arteritis (also known as “giant cell arteritis”); warm autoimmune hemolytic anemia; Wegener's granulomatosis; alopecia universalis; Chagas disease; chronic fatigue syndrome; dysautonomia; endometriosis; hidradenitis suppurativa; interstitial cystitis; neuromyotonia; sarcoidosis; scleroderma; ulcerative colitis; vitiligo; and vulvodynia.

In one embodiment, the inflammatory disease is an inflammatory disease described herein. Examples of inflammatory disease include, but are not limited to: inflammation associated with acne; anemia (e.g., aplastic anemia, haemolytic autoimmune anaemia); asthma; arteritis (e.g., polyarteritis, temporal arteritis, periarteritis nodosa, Takayasu's arteritis); arthritis (e.g., crystalline arthritis, osteoarthritis, psoriatic arthritis, gouty arthritis, reactive arthritis, rheumatoid arthritis and Reiter's arthritis); ankylosing spondylitis; amylosis; amyotrophic lateral sclerosis; allergies or allergic reactions; Alzheimer's disease; atherosclerosis; bronchitis; bursitis; chronic prostatitis; conjunctivitis; Chagas disease; chronic obstructive pulmonary disease; cermatomyositis; diverticulitis; diabetes (e.g., type I diabetes mellitus, type 2 diabetes mellitus); dermatitis; eosinophilic gastrointestinal disorders (e.g., eosinophilic esophagitis, eosinophilic gastritis, eosinophilic gastroenteritis, eosinophilic colitis); eczema; endometriosis; gastrointestinal bleeding; gastritis; gastroesophageal reflux disease (GORD, or its synonym GERD); Guillain-Barre syndrome; infection; ischaemic heart disease; Kawasaki disease; glomerulonephritis; gingivitis; hypersensitivity; headaches (e.g., migraine headaches, tension headaches); ileus (e.g., postoperative ileus and ileus during sepsis); idiopathic thrombocytopenic purpura; interstitial cystitis; inflammatory bowel disease (IBD) (e.g., Crohn's disease, ulcerative colitis, collagenous colitis, lymphocytic colitis, ischaemic colitis, diversion colitis, Behcet's syndrome, indeterminate colitis); inflammatory bowel syndrome (IBS); lupus; multiple sclerosis; morphea; myeasthenia gravis; myocardial ischemia; nephrotic syndrome; pemphigus vulgaris; pernicious anemia; peptic ulcers; psoriasis; polymyositis; primary biliary cirrhosis; Parkinson's disease; pelvic inflammatory disease; reperfusion injury; regional enteritis; rheumatic fever; systemic lupus erythematosus; schleroderma; scierodoma; sarcoidosis; spondyloarthopathies; Sjogren's syndrome; thyroiditis; transplantation rejection; tendonitis; trauma or injury (e.g., frostbite, chemical irritants, toxins, scarring, burns, physical injury); vasculitis; vitiligo; and Wegener's granulomatosis.

Examples of JAK-associated autoimmune and/or inflammatory diseases include, for example, organ transplant rejection (e.g., allograft rejection and graft versus host disease).

Further examples of JAK-associated diseases include autoimmune diseases such as multiple sclerosis, rheumatoid arthritis, juvenile arthritis, psoriasis, type I diabetes, lupus, psoriasis, inflammatory bowel disease, ulcerative colitis, Crohn's disease, myasthenia gravis, immunoglobulin nephropathies, myocarditis, autoimmune thyroid disorders, chronic obstructive pulmonary disease (COPD), and the like. In some embodiments, the autoimmune disease is an autoimmune bullous skin disorder such as pemphigus vulgaris (PV) or bullous pemphigoid (BP).

In one embodiment, the CDP-JAK inhibitor is administered in combination with another therapy, e.g., an autoimmune or inflammatory therapy, e.g., an agent that treats or prevents an autoimmune disorder or inflammatory disorder.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In another aspect, the disclosure features a method of treating a metabolic disorder, in a subject, e.g., a human, the method comprises: administering a composition that comprises a CDP-JAK inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the disorder, to thereby treat the metabolic disorder. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein. Metabolic disorders include disorders, diseases or conditions which are caused or characterized by an abnormal metabolism (i.e., the chemical changes in living cells by which energy is provided for vital processes and activities) in a subject.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

Examples of metablic disorders include, e.g., obesity, diabetes, co-morbidity of obesity disorder, and other obesity-related disorders. The subject to whom the CDP-JAK inhibitor conjugate is administered may be overweight or obese. Alternatively, or in addition, the subject may be diabetic, for example having insulin resistance or glucose intolerance, or both. The subject may have diabetes mellitus, for example, the subject may have Type II diabetes. The subject may be overweight or obese and have diabetes mellitus, for example, Type II diabetes.

In addition, or alternatively, the subject may have, or may be at risk of having, a disorder in which obesity or being overweight is a risk factor. As used herein, “obesity” refers to a body mass index (BMI) of 30 kg/m² or more (National Institute of Health, Clinical Guidelines on the Identification, Evaluation, and Treatment of Overweight and Obesity in Adults (1998)). However, the disclosure is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, all of which are typically referred to as overweight (National Institute of Health, Clinical Guidelines on the Identification, Evaluation, and Treatment of Overweight and Obesity in Adults (1998)). Such disorders include, but are not limited to, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension; hypercholesterolemia; cholithiasis; osteoarthritis; orthopedic injury; insulin resistance, for example, type 2 diabetes and syndrome X; metabolic syndrome; and thromboembolic disease (see Kopelman (2000), Nature 404:635-43; Rissanen et al., British Med. J. 301, 835, 1990).

Other disorders associated with obesity include, but are not limited to, depression, anxiety, panic attacks, migraine headaches, PMS, chronic pain states, fibromyalgia, insomnia, impulsivity, obsessive-compulsive disorder, irritable bowel syndrome (IBS), and myoclonus. Furthermore, obesity is a recognized risk factor for increased incidence of complications of general anesthesia. (See e.g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatititis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odors and infections in the skin folds under the breasts, etc.; large anterior abdominal wall masses, for example abdominal panniculitis with frequent panniculitis, impeding walking, causing frequent infections, odors, clothing difficulties, lower back pain; musculoskeletal disease; pseudo tumor cerebri (or benign intracranial hypertension), and sliding hiatil hernia.

Conditions or disorders associated with increased caloric intake include, but are not limited to, insulin resistance, glucose intolerance, obesity, diabetes, including type 2 diabetes, eating disorders, insulin-resistance syndromes, metabolic syndrome X, and Alzheimer's disease.

In one embodiment, the CDP-JAK inhibitor is administered in combination with another therapy, e.g., metabolic disorder therapy, e.g., an agent that treats or prevents a metabolic disorder.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In another aspect, the disclosure features a method of treating a central nervous system (CNS) disorder, in a subject, e.g., a human, the method comprises: administering a composition that comprises a CDP-JAK inhibitor conjugate, e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the disorder, to thereby treat the CNS disorder. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

Examples of central nervous system disorders include, but are not limited to: a myelopathy; an encephalopathy; central nervous system (CNS) infection; encephalitis (e.g., viral encephalitis, bacterial encephalitis, parasitic encephalitis); meningitis (e.g., spinal meningitis, bacterial meningitis, viral meningitis, fungal meningitis); neurodegenerative diseases (e.g., Huntington's disease; Alzheimer's disease; Parkinson's disease; multiple sclerosis; amyotrophic lateral sclerosis; traumatic brain injury); mental health disorder (e.g., schizophrenia, depression, dementia); pain and addiction disorders; brain tumors (e.g., intra-axial tumors, extra-axial tumors); adult brain tumors (e.g., glioma, glioblastoma); pediatric brain tumors (e.g., medulloblastoma); cognitive impairment; genetic disorders (e.g., Huntington's disease, neurofibromatosis type 1, neurofibromatosis type 2, Tay-Sachs disease, tuberous sclerosis); headache (e.g., tension headache; migraine headache, cluster headache, meningitis headache, cerebral aneurysm and subarachnoid hemorrhage headache, brain tumor headache); stroke (e.g., cerebral ischemia or cerebral infarction, transient ischemic attack, hemorrhagic (e.g., aneurysmal subarachnoid hemorrhage, hypertensive hemorrhage, other sudden hemorrhage)); epilepsy; spinal disease (e.g., degenerative spinal disease (e.g., herniated disc disease, spinal stenosis, and spinal instability), traumatic spine disease; spinal cord trauma; spinal tumors; hydrocephalus (e.g., communicating or non-obstructive hydrocephalus, non-communicating or obstructive hydrocephalus, adult hydrocephalus, pediatric hydrocephalus, normal pressure hydrocephalus, aqueductal stenosis, tumor associated hydrocephalus, pseudotumor cerebri); CNS vasculitis (e.g., primary angiitis of the central nervous system, benign angiopathy of the central nervous system; Arnold Chiari malformation; neuroA/DS; retinal disorders (e.g., age-related macular degeneration, wet age-related macular degeneration, myopic macular degeneration, retinitis pigmentosa, proliferative retinopathies); inner ear disorders; tropical spastic paraparesis; arachnoid cysts; locked-in syndrome; Tourette's syndrome; adhesive arachnoiditis; altered consciousness; autonomic neuropathy; benign essential tremor; brain anomalies; cauda equine syndrome with neurogenic bladder; cerebral edema; cerebral spasticity; cerebral vascular disorder; and Guillain-Barre syndrome.

In one embodiment, the CDP-JAK inhibitor is administered in combination with another therapy, e.g., a central nervious system disorder therapy, e.g., an agent that treats or prevents a central nervous system disorder.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In another aspect, the disclosure features a method of treating neurological deficits, in a subject, e.g., a human, the method comprises: administering a composition that comprises a CDP-JAK inhibitor conjugate, e.g., e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate, to a subject in an amount effective to treat the neurological deficits. In an embodiment, the CDP-JAK inhibitor conjugate comprises a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled, e.g., via a linker such as a linker described herein, to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

Neurological deficits include an impairment or absence of a normal neurological function or presence of an abnormal neurological function. Neurodegeneration of the brain can be the result of disease, injury, and/or aging. Neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

In one embodiment, the CDP-JAK inhibitor is administered in combination with another therapy, e.g., neurological deficit therapy, e.g., an agent that treats or prevents a neurological deficit.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In one aspect, the disclosure features, a method of treating a subject having an autoimmune or inflammatory disorder (e.g., inflammatory bowel disease, psoriasis, rheumatoid arthritis), in a subject, e.g., a human subject. The method comprises administering a CDP-JAK inhibitor conjugate, e.g., a CDP-tofacitinib conjugate, e.g., a CDP-tofacitinib conjugate described herein, e.g., a CDP-tofacitinib conjugate comprising tofacitinib, coupled, e.g., via linkers described herein, to a CDP described herein, to the subject, e.g., human subject, and optionally, providing one or more subsequent administrations of the CDP-JAK inhibitor conjugate, e.g., CDP-tofacitinib conjugate, e.g., CDP-tofacitinib conjugate described herein, e.g., CDP-tofacitinib conjugate comprising tofacitinib, coupled, e.g., via linkers described herein, to a CDP described herein. In one embodiment, the CDP-tofacitinib conjugate comprises tofacitinib coupled via a linker comprising glycine to a CDP described herein. In one embodiment, the CDP-tofacitinib conjugate comprises tofacitinib coupled via a linker comprising hexanoate to a CDP described herein.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

In one embodiment, the CDP-JAK inhibitor, e.g., the CDP-tofacitinib conjugate, is administered by subcutaneous administration. In one embodiment, the CDP-JAK inhibitor, e.g., the CDP-tofacitinib conjugate, is administered by intravenous administration.

In one embodiment, the CDP-JAK inhibitor, e.g., the CDP-tofacitinib conjugate, is administered by subcutaneous administration, and one or more subsequent doses of the CDP-JAK inhibitor, e.g., the CDP-tofacitinib conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-tofacitinib conjugate, is administered at a dose of 1 mg/kg, 2 mg/kg, 3 mg/kg, 4 mg/kg, 5 mg/kg of tofacitinib (wherein the dosage is expressed in mg of drug, as opposed to mg of conjugate). In one embodiment, the method further comprises administering one or more subsequent doses of the CDP-JAK inhibitor conjugate, e.g., the CDP-tofacitinib conjugate, e.g., at a dose of 1 mg/kg, 2 mg/kg, 3 mg/kg, 4 mg/kg, 5 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the CDP-JAK inhibitor conjugate, e.g., the CDP-tofacitinib conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of tofacitinib (wherein the dosage is expressed in mg of drug, as opposed to mg of conjugate). In one embodiment, the method further comprises administering one or more subsequent doses of the CDP-JAK inhibitor conjugate, e.g., the CDP-tofacitinib conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the CDP-JAK inhibitor conjugate, e.g., the CDP-ruxolitinib conjugate, is administered at a dose of 0.05 mg/kg to 2 mg/kg (e.g., 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-pyrrolopyrimidine-containing JAK inhibitor conjugate (e.g., the CDP-ruxolitinib conjugate), e.g., at a dose of 0.05 mg/kg to 2 mg/kg (e.g., 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the inflammatory disorder is psoriasis. In some embodiments, the psoriasis is chronic plaque psoriasis. In some embodiments, the psoriasis is psoriasis vulgaris.

In one embodiment, the autoimmune or inflammatory disorder is arthritis. In some embodiments, the arthritis is ankylosing spondylitis. In some embodiments, the arthritis is juvenile idiopathic arthritis.

In one embodiment, the autoimmune disorder is a autoimmune disorder of the eye, e.g., keratoconjunctivitis sicca.

In one embodiment, the autoimmune disorder is transplantation rejection. In some embodiments, the transplantation rejection is kidney transplantation rejection.

In one embodiment, the autoimmune disorder is inflammatory bowel disease (IBD). In some embodiments, the IBD is Crohn's disease. In some embodiments, the IBD is ulcerative colitis. In some embodiments, the IBD is selected from collagenous colitis, lymphocytic colitis, ischaemic colitis, diversion colitis, Behcet's syndrome, and indeterminate colitis.

In another aspect, the disclosure features, a method of treating rheumatoid arthritis (e.g., moderately to severs active rheumatoid arthritis) in a subject, e.g., a human subject. The method comprises administering a CDP-JAK inhibitor conjugate, e.g., a CDP-tofacitinib conjugate, e.g., a CDP-tofacitinib conjugate described herein, e.g., a CDP-tofacitinib conjugate comprising tofacitinib, coupled, e.g., via linkers described herein, to a CDP described herein, to the subject, e.g., human subject, and optionally, providing one or more subsequent administrations of the CDP-JAK inhibitor conjugate, e.g., CDP-tofacitinib conjugate, e.g., CDP-tofacitinib conjugate described herein, e.g., CDP-tofacitinib conjugate comprising tofacitinib, coupled, e.g., via linkers described herein, to a CDP described herein, to thereby treat the rheumatoid arthritis.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

In one embodiment, the CDP-JAK inhibitor, e.g., the CDP-tofacitinib conjugate is administered by subcutaneous administration. In one embodiment, the CDP-JAK inhibitor, e.g., the CDP-tofacitinib conjugate is administered by intravenous administration.

In one embodiment, the CDP-JAK inhibitor, e.g., the CDP-tofacitinib conjugate, is administered by subcutaneous administration, and one or more subsequent doses of the CDP-JAK inhibitor, e.g., the CDP-tofacitinib conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-tofacitinib conjugate, is administered at a dose of 1 mg/kg, 2 mg/kg, 3 mg/kg, 4 mg/kg, 5 mg/kg of tofacitinib (wherein the dosage is expressed in mg of drug, as opposed to mg of conjugate). In one embodiment, the method further comprises administering one or more subsequent doses of the CDP-JAK inhibitor conjugate, e.g., the CDP-tofacitinib conjugate, e.g., at a dose of 1 mg/kg, 2 mg/kg, 3 mg/kg, 4 mg/kg, 5 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the CDP-JAK inhibitor conjugate, e.g., the CDP-tofacitinib conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of tofacitinib (wherein the dosage is expressed in mg of drug, as opposed to mg of conjugate). In one embodiment, the method further comprises administering one or more subsequent doses of the CDP-JAK inhibitor conjugate, e.g., the CDP-tofacitinib conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the subject has previously been treated with an antimetabolite, e.g., an antifolate, e.g., methotrexate. In one embodiment, the subject is methotrexate-sensitive, or the rheumatoid arthritis is resistant to, and/or has relapsed after treatment with methotrexate.

In one embodiment, the CDP-JAK inhibitor conjugate is administered in combination with an antirheumatic agent, e.g., an antimetabolite, e.g., an antifolate, e.g., methotrexate, and/or other disease-modifying antirheumatic drug (DMARD).

In one aspect, the disclosure features, a method of treating an autoimmune or inflammatory disorder (e.g., rheumatoid arthritis or psoriasis), in a subject, e.g., a human subject. The method comprises administering a CDP-JAK inhibitor conjugate, e.g., a CDP-ruxolitinib conjugate, e.g., a CDP-ruxolitinib conjugate described herein, e.g., a CDP-ruxolitinib conjugate comprising ruxolitinib, coupled, e.g., via linkers described herein, to a CDP described herein, to the subject, e.g., human subject, and optionally, providing one or more subsequent administrations of the CDP-JAK inhibitor conjugate, e.g., CDP-ruxolitinib conjugate, e.g., CDP-ruxolitinib conjugate described herein, e.g., CDP-ruxolitinib conjugate comprising ruxolitinib, coupled, e.g., via linkers described herein, to a CDP described herein, to thereby treat the autoimmune or inflammatory disorder (e.g., rheumatoid arthritis or psoriasis).

In one embodiment, the CDP-JAK inhibitor, e.g., the CDP-ruxolitinib conjugate is administered by subcutaneous administration. In one embodiment, the CDP-JAK inhibitor, e.g., the CDP-ruxolitinib conjugate is administered by intravenous administration.

In one embodiment, the CDP-JAK inhibitor, e.g., the CDP-ruxolitinib conjugate, is administered by subcutaneous administration, and one or more subsequent doses of the CDP-JAK inhibitor, e.g., the CDP-ruxolitinib conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-ruxolitinib conjugate, is administered at a dose of 5 mg/kg, 10 mg/kg, 15 mg/kg, 20 mg/kg, 25 mg/kg of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-ruxolitinib conjugate, e.g., at a dose of 5 mg/kg, 10 mg/kg, 15 mg/kg, 20 mg/kg, 25 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder (e.g., rheumatoid arthritis or psoriasis). In one embodiment, each subsequent dose is one week, two weeks, three weeks or four weeks after the previous dose.

In one embodiment, the CDP-JAK inhibitor conjugate, e.g., the CDP-ruxolitinib conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-ruxolitinib conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder (e.g., rheumatoid arthritis or psoriasis). In one embodiment, each subsequent dose is one week, two weeks, three weeks or four weeks after the previous dose.

In one embodiment, the autoimmune or inflammatory disorder is psoriasis, e.g., plaque psoriasis.

In one embodiment, the autoimmune disorder is rheumatoid arthritis.

In another aspect, the disclosure features, a method of treating a proliferative disorder, e.g., a cancer, in a subject, e.g., a human subject. The method comprises administering a CDP-JAK inhibitor conjugate, e.g., a CDP-ruxolitinib conjugate, e.g., a CDP-ruxolitinib conjugate described herein, e.g., a CDP-ruxolitinib conjugate comprising ruxolitinib, coupled, e.g., via linkers described herein, to a CDP described herein, to the subject, e.g., human subject, and optionally, providing one or more subsequent administrations of the CDP-JAK inhibitor conjugate, e.g., CDP-ruxolitinib conjugate, e.g., CDP-ruxolitinib conjugate described herein, e.g., CDP-ruxolitinib conjugate comprising ruxolitinib, coupled, e.g., via linkers described herein, to a CDP described herein, to thereby treat the proliferative disorder, e.g., cancer.

In one embodiment, the cancer is resistant to gemcitabine, e.g., gemcitabine resistant pancreatic cancer.

In one embodiment, the CDP-JAK inhibitor, e.g., the CDP-ruxolitinib conjugate is administered by subcutaneous administration. In one embodiment, the CDP-JAK inhibitor, e.g., the CDP-ruxolitinib conjugate is administered by intravenous administration.

In one embodiment, the CDP-JAK inhibitor, e.g., the CDP-ruxolitinib conjugate, is administered by subcutaneous administration, and one or more subsequent doses of the CDP-JAK inhibitor, e.g., the CDP-ruxolitinib conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-ruxolitinib conjugate, is administered at a dose of 5 mg/kg, 10 mg/kg, 15 mg/kg, 20 mg/kg, 25 mg/kg of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-ruxolitinib conjugate, e.g., at a dose of 5 mg/kg, 10 mg/kg, 15 mg/kg, 20 mg/kg, 25 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the proliferative disorder, e.g., cancer. In one embodiment, each subsequent dose is one week, two weeks, three weeks or four weeks after the previous dose.

In one embodiment, the CDP-JAK inhibitor conjugate, e.g., the CDP-ruxolitinib conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of ruxolitinib (wherein the dosage is expressed in mg of drug, as opposed to mg of conjugate). In one embodiment, the method further comprises administering one or more subsequent doses of the CDP-JAK inhibitor conjugate, e.g., the CDP-ruxolitinib conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the proliferative disorder, e.g., cancer. In one embodiment, each subsequent dose is one week, two weeks, three weeks or four weeks after the previous dose.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia. In one embodiment, the subject has previously been treated with an anticancer agent, e.g., a tyrosine kinase inhibitor (e.g., imatinib, dasatinib, nilotinib). In one embodiment, the subject is tyrosine kinase inhibitor-sensitive, or the leukemia is resistant to, and/or has relapsed after treatment with a tyrosine kinase inhibitor (e.g., imatinib, dasatinib, nilotinib).

In one embodiment, the method comprises administering the CDP-JAK inhibitor conjugate, e.g., the CDP-ruxolitinib conjugate, in combination with another anticancer agent, e.g., a tyrosine kinase inhibitor, (e.g., imatinib, dasatinib, nilotinib), to treat the cancer, e.g., the leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In one embodiment, the method comprises administering the CDP-JAK inhibitor conjugate, e.g., the CDP-ruxolitinib conjugate, in combination with another anticancer agent, e.g., thalidomide derivative (e.g., lenalidomide) and/or a histone deacetylase (HDAC) inhibitor (e.g., panobinostat) to treat the cancer, e.g., the myelofibrosis.

In some embodiments, the myeloproliferative disorder is multiple myeloma.

In one embodiment, the cancer is a solid tumor, e.g., breast cancer, prostate cancer, or pancreatic cancer. In some embodiments, the cancer is prostate cancer, e.g., hormone refractory prostate cancer. In some embodiments, the cancer is pancreatic cancer, e.g., metastatic pancreatic adenocarcinoma. In some embodiments, the cancer is breast cancer, e.g., estrogen receptor positive breast cancer, estrogen receptor negative breast cancer, HER-2 positive breast cancer, HER-2 negative breast cancer, triple negative breast cancer or inflammatory breast cancer.

In one embodiment, the method comprises administering the CDP-JAK inhibitor conjugate, e.g., the CDP-ruxolitinib conjugate, in combination with another anticancer agent, e.g., an antimetabolite, e.g., pyrimidine analog (e.g., capecitabine, cytrarabine, gemcitabine, 5-fluorouracil) and/or a taxane (e.g., docetaxel, paclitaxel, cabazitaxel, larotaxel).

In another aspect, the disclosure features, a method of treating an autoimmune or inflammatory disorder (e.g., rheumatoid arthritis or psoriasis), in a subject, e.g., a human subject. The method comprises administering a CDP-JAK inhibitor conjugate, e.g., a CDP-baricitinib conjugate, e.g., a CDP-baricitinib conjugate described herein, e.g., a CDP-baricitinib conjugate comprising baricitinib, coupled, e.g., via linkers described herein, to a CDP described herein, to the subject, e.g., human subject, and optionally, providing one or more subsequent administrations of the CDP-JAK inhibitor conjugate, e.g., CDP-baricitinib conjugate, e.g., CDP-baricitinib conjugate described herein, e.g., CDP-baricitinib conjugate comprising baricitinib, coupled, e.g., via linkers described herein, to a CDP described herein, to thereby treat the autoimmune or inflammatory disorder.

In one embodiment, the CDP-JAK inhibitor, e.g., the CDP-baricitinib conjugate is administered by subcutaneous administration. In one embodiment, the CDP-JAK inhibitor, e.g., the CDP-baricitinib conjugate, is administered by intravenous administration.

In one embodiment, the CDP-JAK inhibitor, e.g., the CDP-baricitinib conjugate, is administered by subcutaneous administration, and one or more subsequent doses of the CDP-JAK inhibitor, e.g., the CDP-baricitinib conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-baricitinib conjugate, is administered at a dose of 1 mg/kg, 2 mg/kg, 3 mg/kg, 4 mg/kg, 5 mg/kg of baricitinib (wherein the dosage is expressed in mg of drug, as opposed to mg of conjugate). In one embodiment, the method further comprises administering one or more subsequent doses of the CDP-JAK inhibitor conjugate, e.g., the CDP-baricitinib conjugate, e.g., at a dose of 1 mg/kg, 2 mg/kg, 3 mg/kg, 4 mg/kg, 5 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the CDP-JAK inhibitor conjugate, e.g., the CDP-baricitinib conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of baricitinib (wherein the dosage is expressed in mg of drug, as opposed to mg of conjugate). In one embodiment, the method further comprises administering one or more subsequent doses of the CDP-JAK inhibitor conjugate, e.g., the CDP-baricitinib conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the autoimmune or inflammatory disorder is rheumatoid arthritis.

In one embodiment, the autoimmune disorder is diabetic kidney disease.

In one embodiment, the autoimmune disorder is an autoinflammatory syndrome, e.g., chronic atypical neutrophilic dermatosis.

In one embodiment, the autoimmune or inflammatory disorder is psoriasis.

In one embodiment, the method comprises selecting a subject, e.g., a human subject, e.g., a patient, on the basis of having or at risk of developing certain disorders, e.g., a malignancy (e.g., other than a successfully treated non-melanoma skin cancer (NMSC)), renal and/or hepatic impairment, lymphopenia (e.g., a lymphocyte count less than about 500 cells/mm³), neutropenia (e.g., an absolute neutrophil count (ANC) of less than 500 cells/mm³), anemia (e.g., a greater than 2 g/dL decrease or less than 8.0 g/dL in hemoglobin levels), serious infections (e.g., due to bacterial, mycobacterial, fungal, or viral infections), elevated liver enzymes, elevated lipid levels, or a gastrointestinal performation (e.g., due to diverticulitis).

In another aspect, the disclosure features, a method of treating an autoimmune or inflammatory disorder (e.g., rheumatoid arthritis), in a subject, e.g., a human subject. The method comprises administering a CDP-JAK inhibitor conjugate, e.g., a CDP-GLPG0634 conjugate, e.g., a CDP-GLPG0634 conjugate described herein, e.g., a CDP-GLPG0634 conjugate comprising GLPG0634, coupled, e.g., via linkers described herein, to a CDP described herein, to the subject, e.g., human subject, and optionally, providing one or more subsequent administrations of the CDP-JAK inhibitor conjugate, e.g., CDP-GLPG0634 conjugate, e.g., CDP-GLPG0634 conjugate described herein, e.g., CDP-GLPG0634 conjugate comprising GLPG0634, coupled, e.g., via linkers described herein, to a CDP described herein, to thereby treat the autoimmune or inflammatory disorder.

In one embodiment, the CDP-JAK inhibitor, e.g., the CDP-GLPG0634 conjugate is administered by subcutaneous administration. In one embodiment, the CDP-JAK inhibitor, e.g., the CDP-GLPG0634 conjugate is administered by intravenous administration.

In one embodiment, the CDP-JAK inhibitor, e.g., the CDP-GLPG0634 conjugate, is administered by subcutaneous administration, and one or more subsequent doses of the CDP-JAK inhibitor, e.g., the CDP-GLPG0634 conjugate is administered one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose.

In one embodiment, the CDP-JAK inhibitor conjugate, e.g., the CDP-GLPG0634 conjugate, is administered at a dose of 1 mg/kg, 2 mg/kg, 3 mg/kg, 4 mg/kg, 5 mg/kg of GLPG0634 (wherein the dosage is expressed in mg of drug, as opposed to mg of conjugate). In one embodiment, the method further comprises administering one or more subsequent doses of the CDP-JAK inhibitor conjugate, e.g., the CDP-GLPG0634 conjugate, e.g., at a dose of 1 mg/kg, 2 mg/kg, 3 mg/kg, 4 mg/kg, 5 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the CDP-JAK inhibitor conjugate, e.g., the CDP-GLPG0634 conjugate, is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of GLPG0634 (wherein the dosage is expressed in mg of drug, as opposed to mg of conjugate). In one embodiment, the method further comprises administering one or more subsequent doses of the CDP-JAK inhibitor conjugate, e.g., the CDP-GLPG0634 conjugate, e.g., at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg. In one embodiment, each subsequent dose is administered, independently, one week (e.g., 5, 6, 7, 8, 9 days) after the previous dose, two weeks (e.g., 12, 13, 14, 15, 16 days) after the previous dose, three weeks (e.g., 19, 20, 21, 22, 23 days) after the previous dose, four weeks (e.g., 26, 27, 28, 29, 30, 31 days) after the previous dose, five weeks (e.g., 33, 34, 35, 36, 37, 38 days) after the previous dose, 6 weeks (e.g., 40, 41, 42, 43, 44 days) after the previous dose, seven weeks (e.g., 47, 48, 49, 50 or 51 days) after the previous dose, or eight weeks (e.g., 54, 55, 56, 57, 58 days) after the previous dose, e.g., the initial, administration, to thereby treat the autoimmune or inflammatory disorder. In one embodiment, each subsequent dose is one week, two weeks, three weeks or four weeks after the previous dose.

In one embodiment, the method comprises selecting a subject, e.g., a human subject, e.g., a patient, on the basis of having or at risk of developing certain disorders, e.g., a malignancy, e.g., other than a successfully treated non-melanoma skin cancer (NMSC), renal or hepatic impairment, lymphopenia, e.g., a lymphocyte count less than about 500 cells/mm³, neutropenia, e.g., an absolute neutrophil count (ANC) of less than 500 cells/mm³, anemia, e.g., a greater than 2 g/dL decrease or less than 8.0 g/dL in hemoglobin levels, serious infections, e.g., due to bacterial, mycobacterial, fungal, or viral infections, elevated liver enzymes, elevated lipid levels, or gastrointestinal performations, e.g., due to diverticulitis.

In another aspect, the disclosure features a method of identifying a subject, e.g., a human, having a proliferative disorder, e.g., cancer, for treatment with a e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, the method comprising identifying a subject having a proliferative disorder who has received an anticancer agent; and administering a composition comprising a e.g., CDP-JAK inhibitor conjugate described herein, e.g., a CDP-JAK1, -JAK2, -JAK3, and/or -Tyk2 inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, to a subject, e.g., a human, in an amount effective to treat the disorder, to thereby treat the proliferative disorder.

In another aspect, the disclosure features a method of treating a proliferative disorder, e.g., a cancer in a subject, e.g., a human, the method comprising:

providing a subject who has a proliferative disorder, e.g., cancer, and has been treated with a chemotherapeutic agent that did not effectively treat the proliferative disorder, e.g., cancer (e.g., the subject has a chemotherapeutic refractory cancer, a chemotherapeutic resistant cancer and/or a relapsed cancer) or who had an unacceptable side effect (e.g., the subject has a chemotherapeutic sensitive cancer), and

administering a CDP-JAK inhibitor conjugate, e.g., a CDP-JAK inhibitor conjugate described herein, to a subject in an amount effective to treat the proliferative disorder, e.g., cancer, to thereby treat the proliferative disorder, e.g., cancer.

In one embodiment, the cancer is resistant to gemcitabine, e.g., gemcitabine resistant pancreatic cancer.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In one embodiment, the CDP-JAK inhibitor conjugate comprises a pyrrolopyrimidine-containing JAK inhibitor (e.g., tofacitinib, ruxolitinib, baricitinib or GLPG0634), and the CDP-pyrrolopyrimidine-containing JAK inhibitor conjugate is administered at a dose and/or dosing schedule described herein.

In another aspect, the disclosure features a method of identifying a subject, e.g., a human, having a proliferative disorder, e.g., cancer, for treatment with a CDP-JAK inhibitor conjugate, e.g., a CDP-JAK inhibitor conjugate described herein, e.g., a CDP-ruxolitinib conjugate, the method comprising

identifying a subject having a proliferative disorder who has received an anticancer agent (e.g., a JAK inhibitor) and has a neutrophil count and/or a platelet count less than a standard; and

identifying the subject as suitable for treatment with a CDP-JAK inhibitor conjugate, e.g., a CDP-JAK inhibitor conjugate described herein, e.g., a CDP-ruxolitinib conjugate described herein.

In one embodiment, the method further comprising administering a CDP-JAK inhibitor conjugate, e.g., a CDP-JAK inhibitor conjugate described herein, e.g., a CDP-ruxolitinib conjugate described herein in an amount effective to treat the disorder.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In one embodiment, the cancer is a cancer described herein.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In another embodiment, the cancer is a cancer known to have a high frequency of mutations in JAK2, e.g., a V617F mutation.

In another embodiment, the cancer is a cancer known to have JAK2 gene fusions, e.g., such as in leukemia patients.

In another embodiment, the proliferative disorder is a myeloproliferative disorder, e.g., polycythemia vera, essential thrombocytosis, myelofibrosis, or myelosclerosis.

In one embodiment, the CDP-JAK inhibitor conjugate is administered in combination with one or more additional chemotherapeutic agent, e.g., a chemotherapeutic agent or combination of chemotherapeutic agents described herein. In one embodiment, the CDP-JAK inhibitor conjugate is administered in combination with a granulocyte colony stimulating factor, e.g., GCSF or GMCSF.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In one embodiment, the standard is a platelet count below 50×10⁹/L.

In another aspect, the disclosure features a method of treating a subject, e.g., a human, with a proliferative disorder, e.g., cancer, the method comprising

selecting a subject having a proliferative disease who has received an anticancer agent (e.g., a JAK inhibitor) and has a neutrophil count and/or platelet count less than a standard; and

administering a CDP-JAK inhibitor conjugate, e.g., a CDP-JAK inhibitor conjugate described herein, e.g., a CDP-ruxolitinib conjugate described herein, to the subject in an amount effective to treat the proliferative disorder, to thereby treat the disorder.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In one embodiment, the cancer is a cancer described herein.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In another embodiment, the cancer is a cancer known to have a high frequency of mutations in JAK2, e.g., a V617F mutation.

In another embodiment, the cancer is a cancer known to have JAK2 gene fusions, e.g., such as in leukemia patients.

In another embodiment, the proliferative disorder is a myeloproliferative disorder, e.g., polycythemia vera, essential thrombocytosis, myelofibrosis, or myelosclerosis.

In one embodiment, the CDP-JAK inhibitor conjugate is administered in combination with one or more additional chemotherapeutic agent, e.g., a chemotherapeutic agent or combination of chemotherapeutic agents described herein. In one embodiment, the CDP-JAK inhibitor conjugate is administered in combination with a granulocyte colony stimulating factor, e.g., GCSF or GMCSF.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In one embodiment, the standard is a platelet count below 50×10⁹/L.

In another aspect, the disclosure features a method for selecting a subject, e.g., a human, with a proliferative disorder, e.g., cancer, for treatment with a CDP-JAK inhibitor conjugate, e.g., a CDP-JAK inhibitor conjugate described herein, e.g., a CDP-ruxolitinib conjugate described herein, comprising:

determining whether a subject with a proliferative disorder has moderate to severe neutropenia; and

selecting a subject for treatment with a CDP-JAK inhibitor conjugate, e.g., a CDP-ruxolitinib conjugate, on the basis that the subject has moderate to severe neutropenia.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In one embodiment, the cancer is a cancer described herein.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In another embodiment, the cancer is a cancer known to have a high frequency of mutations in JAK2, e.g., a V617F mutation.

In another embodiment, the cancer is a cancer known to have JAK2 gene fusions, e.g., such as in leukemia patients.

In another embodiment, the proliferative disorder is a myeloproliferative disorder, e.g., polycythemia vera, essential thrombocytosis, myelofibrosis, or myelosclerosis.

In one embodiment, the method further comprises administering a CDP-JAK inhibitor conjugate, e.g., a CDP-ruxolitinib conjugate described herein, to the subject.

In one embodiment, the standard for moderate neutropenia is a neutrophil count of 1000 to 500 cells/mm³. In one embodiment, the standard for severe neutropenia is a neutrophil count of less than 500 cells/mm³.

In another aspect, the disclosure features a method for treating a subject, e.g., a human, with a proliferative disorder, e.g., cancer, comprising:

selecting a subject with a proliferative disorder, e.g., cancer, who has moderate to severe neutropenia; and

administering a CDP-JAK inhibitor conjugate, e.g., a CDP-JAK inhibitor conjugate described herein, e.g., a CDP-ruxolitinib conjugate described herein, to the subject in an amount effective to treat the disorder, to thereby treat the proliferative disorder.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In one embodiment, the cancer is a cancer described herein.

In one embodiment, the cancer is a leukemia. In some embodiments, the leukemia is chronic myeloid leukemia (CML). In some embodiments, the leukemia is acute lymphoblastic leukemia, acute lymphoblastic leukemia, acute myeloid leukemia, or chronic lymphocytic leukemia. In some embodiments, the leukemia is chronic phase chronic myeloid leukemia. In some embodiments, the leukemia is myelomonocytic leukemia.

In one embodiment, the cancer is a lymphoma. In some embodiments, the lymphoma is relapsed or refractory diffuse large B-cell, or peripheral T-cell non-Hodgkin lymphoma.

In one embodiment, the cancer is a cancer of the bone marrow, e.g., a myeloproliferative disorder, e.g., a myelofibrosis. In some embodiments, the myelofibrosis is primary or secondary myelofibrosis, thrombocythemia, e.g., post essential thrombocythemia-myelofibrosis, or post polycythemia vera-myelofibrosis.

In another embodiment, the cancer is a cancer known to have a high frequency of mutations in JAK2, e.g., a V617F mutation.

In another embodiment, the cancer is a cancer known to have JAK2 gene fusions, e.g., such as in leukemia patients.

In another embodiment, the proliferative disorder is a myeloproliferative disorder, e.g., polycythemia vera, essential thrombocytosis, myelofibrosis, or myelosclerosis.

In one embodiment, the method further comprises administering a CDP-JAK inhibitor conjugate, e.g., a CDP-ruxolitinib conjugate described herein, to the subject.

In one embodiment, the standard for moderate neutropenia is a neutrophil count of 1000 to 500 cells/mm³. In one embodiment, the standard for severe neutropenia is a neutrophil count of less than 500 cells/mm³.

In another aspect, the disclosure features a method for selecting a subject, e.g., a human, with a proliferative disorder, e.g., cancer, an autoimmune disorder or an inflammatory disorder, for treatment with a CDP-JAK inhibitor conjugate, e.g., a CDP-JAK inhibitor conjugate described herein, e.g., a CDP-tofacitinib conjugate described herein, comprising:

determining whether a subject with a proliferative disorder, e.g., cancer, an autoimmune disorder or an inflammatory disorder has an infection (e.g., tuberculosis, bacterial, invasive fungal, viral or other opportunistic infection); and

selecting a subject for treatment with a CDP-JAK inhibitor conjugate, e.g., a CDP-JAK inhibitor conjugate described herein, on the basis that the subject has an infection.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In another aspect, the disclosure features a method for treating a subject, e.g., a human, with a proliferative disorder, e.g., cancer, an autoimmune disorder or an inflammatory disorder, comprising:

selecting a subject with a proliferative disorder, e.g., cancer, an autoimmune disorder or an inflammatory disorder who has an infection (e.g., tuberculosis, bacterial, invasive fungal, viral or other opportunistic infection); and

administering a CDP-JAK inhibitor conjugate, e.g., a CDP-JAK inhibitor conjugate described herein, e.g., a CDP-tofacitinib conjugate described herein, to the subject in an amount effective to treat the disorder, to thereby treat the proliferative disorder, the autoimmune disorder or the inflammatory disorder.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In another aspect, the disclosure features a method for selecting a subject, e.g., a human, with a proliferative disorder, e.g., cancer, an autoimmune disorder or an inflammatory disorder, for treatment with a CDP-JAK inhibitor conjugate, e.g., a CDP-JAK inhibitor conjugate described herein, e.g., a CDP-tofacitinib conjugate described herein, comprising:

determining whether a subject with a proliferative disorder, e.g., cancer, an autoimmune disorder or an inflammatory disorder has a gastrointestinal perforation; and

selecting a subject for treatment with a CDP-JAK inhibitor conjugate, e.g., a CDP-JAK inhibitor conjugate described herein, on the basis that the subject has a gastrointestinal perforation.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In another aspect, the disclosure features a method for treating a subject, e.g., a human, with a proliferative disorder, e.g., cancer, an autoimmune disorder or an inflammatory disorder, comprising:

selecting a subject with a proliferative disorder, e.g., cancer, an autoimmune disorder or an inflammatory disorder who has a gastrointestinal perforation; and

administering a CDP-JAK inhibitor conjugate, e.g., a CDP-JAK inhibitor conjugate described herein, e.g., a CDP-tofacitinib conjugate described herein, to the subject in an amount effective to treat the disorder, to thereby treat the proliferative disorder, the autoimmune disorder or the inflammatory disorder.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In another aspect, the disclosure features a method of selecting a subject, e.g., a human, with a proliferative disorder, e.g., cancer, an autoimmune disorder or an inflammatory disorder for treatment with a CDP-JAK inhibitor conjugate, e.g., a CDP-JAK inhibitor described herein, e.g., a CDP-ruxolitinib conjugate, and/or a CDP-tofacitinib conjugate described herein, comprising:

determining if a subject has hepatic impairment, e.g., if the subject has alanine aminotransferase (ALT), aspartate aminotransferase (AST) and/or bilirubin levels in a subject having a proliferative disorder, an autoimmune disorder or an inflammatory disorder; and

selecting a subject having hepatic impairment, e.g., a subject having ALT and/or AST levels greater than 1.5 times the upper limit of normal (ULN) (e.g., 2.5 times greater than the ULN) and/or bilirubin levels greater than 1.5 or 2 times the ULN for treatment with a CDP-JAK inhibitor conjugate, e.g., a CDP-JAK inhibitor described herein, e.g., a CDP-ruxolitinib conjugate, and/or a CDP-tofacitinib conjugate described herein.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In another aspect, the disclosure features a method of treating a subject, e.g., a human, having a proliferative disorder, e.g., cancer, an autoimmune disorder or an inflammatory disorder, comprising:

selecting a subject with a proliferative disorder who has hepatic impairment, e.g., a subject who has alanine aminotransferase (ALT) and/or aspartate aminotransferase (AST) levels greater than 1.5 times the upper limit of normal (ULN) (e.g., 2.5 times the ULN) and/or bilirubin levels greater than 1.5 or 2 times the ULN; and

administering a CDP-JAK inhibitor conjugate, e.g., a CDP-JAK inhibitor described herein, e.g., a CDP-ruxolitinib conjugate, and/or a CDP-tofacitinib conjugate described herein, to the subject in an amount effective to treat the disorder, to thereby treat the proliferative disorder, the autoimmune disorder or the inflammatory disorder.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In another aspect, the disclosure features a method of selecting a subject, e.g., a human, with a proliferative disorder, e.g., cancer, an autoimmune disorder or an inflammatory disorder for treatment with a CDP-JAK inhibitor conjugate, e.g., a CDP-JAK inhibitor described herein, e.g., a CDP-ruxolitinib conjugate, and/or a CDP-tofacitinib conjugate described herein, comprising:

determining if a subject has hepatic impairment, e.g., the subject has alkaline phosphatase (ALP), serum glutamate oxaloacetate transaminase (SGOT), serum glutamate pyruvate transaminase (SGPT) and/or bilirubin levels in a subject having a proliferative disorder, an autoimmune disorder or an inflammatory disorder; and

selecting a subject having hepatic impairment, e.g., a subject having ALP levels greater than 2.5 times the upper limit of normal (ULN), SGOT and/or SGPT levels greater than 1.5 times the upper limit of normal (ULN) and/or bilirubin levels greater than the ULN for treatment with a CDP-JAK inhibitor conjugate, e.g., a CDP-JAK inhibitor described herein, e.g., a CDP-ruxolitinib conjugate, and/or a CDP-tofacitinib conjugate described herein.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In another aspect, the disclosure features a method of treating a subject, e.g., a human, having a proliferative disorder, e.g., cancer, an autoimmune disorder or an inflammatory disorder, comprising:

selecting a subject with a proliferative disorder, an autoimmune disorder or an inflammatory disorder who has hepatic impairment, e.g., a subject who has alkaline phosphatase (ALP) levels greater than 2.5 times the upper limit of normal (ULN), serum glutamate oxaloacetate transaminase (SGOT) and/or serum glutamate pyruvate transaminase (SGPT) levels greater than 1.5 times the ULN and/or bilirubin levels greater than the ULN; and

administering a CDP-JAK inhibitor conjugate, e.g., a CDP-JAK inhibitor described herein, e.g., a CDP-ruxolitinib conjugate, and/or a CDP-tofacitinib conjugate described herein, to the subject in an amount effective to treat the disorder, to thereby treat the proliferative disorder, the autoimmune disorder or the inflammatory disorder.

In an embodiment, the CDP-JAK inhibitor conjugate comprises a JAK inhibitor molecules (e.g., tofacitinib), coupled, e.g., via a linker such as a linker described herein, to a CDP moiety, e.g., a CDP described herein. In an embodiment, the CDP-JAK inhibitor conjugate comprises a JAK inhibitor (e.g., tofacitinib), coupled via a linker described herein to a CDP moiety, e.g., a CDP described herein.

In one embodiment, the CDP-tofacitinib conjugate is administered at a dose and/or dosing schedule described herein.

In an embodiment, the CDP-JAK inhibitor conjugate comprises a JAK inhibitor molecules (e.g., ruxolitinib), coupled, e.g., via a linker such as a linker described herein, to a CDP moiety, e.g., a CDP described herein. In an embodiment, the CDP-JAK inhibitor conjugate comprises a JAK inhibitor (e.g., ruxolitinib), coupled via a linker described herein to a CDP moiety, e.g., a CDP described herein.

In one embodiment, the CDP-ruxolitinib conjugate is administered at a dose and/or dosing schedule described herein.

In one embodiment, the CDP-JAK inhibitor conjugate, e.g., the CDP-JAK inhibitor conjugate described herein, is formulated for subcutaneous administration. In one embodiment, the subcutaneous formulation comprising the CDP-JAK inhibitor conjugate is a sterile, preservative-free solution that includes the CDP-JAK inhibitor conjugate. In one embodiment, the disclosure features an article of manufacture, e.g., a device described herein (e.g., a syringe or injector pen for subcutaneous administration) that contains a subcutaneous formulation comprising a CDP-JAK inhibitor conjugate described herein. In one embodiment, the article of manufacture is a single-use, prefilled pen or as a single-use, prefilled glass syringe (e.g., a pen or syringe described herein. In one embodiment, the article of manufacture is filled with 1 mL of a subcutaneous formulation comprising the CDP-JAK inhibitor conjugate. In one embodiment, the subcutaneous formulation includes in an amount of CDP-JAK inhibitor conjugate such that 15 mg, 20 mg, 25, mg, 30 mg, 35 mg, 40 mg, 45 mg or 50 mg of the JAK inhibitor is present in the formulation.

In one aspect, the disclosure features a method of making a CDP-JAK inhibitor conjugate described herein. In some embodiments, the method comprises making a CDP-JAK inhibitor conjugate by conjugating a plurality of JAK inhibitors to the CDP. In embodiments, the resulting CDP-JAK inhibitor conjugate includes a plurality of JAK inhibitors. In embodiments, less than 100% of the available positions on the CDP are reacted with a JAK inhibitor. In some embodiments, the method comprises a reacting cyclodextrin containing monomers and comonomers, wherein either the cyclodextrin containing monomers or the comonomers include a JAK inhibitor attached thereto to form a CDP-JAK inhibitor conjugate. Exemplary methods are described herein.

In one aspect, the disclosure features a method of making a nanoparticle comprising a CDP-JAK inhibitor conjugate described herein. In embodiments, a composition comprising a CDP-JAK inhibitor conjugate (e.g., a reaction mixture) is contacted with an antisolvent (e.g., a solvent in which the CDP-JAK inhibitor conjugate is not soluble), thereby producing a nanoparticle comprising a CDP-JAK inhibitor conjugate. In some embodiments, the method further comprises filtering the nanoparticle.

In one aspect, the disclosure features a method of formulating a CDP-JAK inhibitor conjugate or a nanoparticle comprising a CDP-JAK inhibitor conjugate into a composition such as a pharmaceutical composition described herein. The method comprises combining a CDP-JAK inhibitor conjugate or a nanoparticle comprising a CDP-JAK inhibitor conjugate with a pharmaceutically acceptable excipient. In some embodiments, the composition is formulated for IV or subcutaneous administration.

In another aspect, the disclosure features, a method of evaluating a particle or a preparation of particles, wherein said particles, comprise one or a plurality of CDP therapeutic agent conjugate molecules, e.g., CDP-JAK inhibitor conjugates, e.g., CDP-JAK inhibitor conjugates described herein. The method comprises:

providing a sample comprising one or a plurality of said particles;

determining a value for the number of CDP-conjugate molecules in a particle in said sample (the conjugate number),

thereby evaluating a preparation of particles.

In an embodiment the method comprises one or both of:

a) comparing said determined value with a reference value, e.g., a range of values, or

b) responsive to said determination, classifying said particles,

In an embodiment the particle is a nanoparticle.

In an embodiment the method further comprises comparing said determined value with a reference standard. In an embodiment the reference value can be selected from a value, e.g., a range, provided herein, e.g., 1 or 2 to 8, 1 or 2 to 7, 1 or 2 to 6, 1 or 2 to 5, or 2-4.

In an embodiment the reference value can be selected from a value, e.g., a range, provided herein, e.g., 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP-therapeutic agent (e.g., JAK inhibitor) conjugate is selected from those disclosed in herein.

In an embodiment said therapeutic agent (e.g., JAK inhibitor is selected from those disclosed herein.

In an embodiment said particle is selected from those disclosed in herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

In another aspect, the disclosure features, a particle, e.g., a nanoparticle, comprising one or more CDP-therapeutic agent (e.g., JAK inhibitor) conjugates described herein, having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

The details of one or more embodiments of the disclosure are set forth in the description below. Other features, objects, and advantages of the disclosure will be apparent from the description and the drawings, and from the claims.

### BRIEF DESCRIPTION OF THE FIGURES

FIGS. 1-11 depict exemplary CDP-JAK inhibitor conjugates.

FIG. 1 depicts a CDP-tofacitinib conjugate.

FIG. 2 depicts a CDP-ruxolitinib conjugate.

FIG. 3 depicts a CDP-baricitinib conjugate.

FIG. 4 depicts a CDP-lestauritinib conjugate.

FIG. 5 depicts a CDP-pacritinib conjugate.

FIG. 6 depicts a CDP-CYT387 conjugate.

FIG. 7 depicts a CDP-XL019 conjugate.

FIG. 8 depicts a CDP-INCB16562 conjugate.

FIG. 9 depicts a AZD1480 conjugate.

FIG. 10 depicts a CDP-TG101348 conjugate.

FIG. 11 depicts a CDP-NVP-BSK805 conjugate.

FIG. 12 depicts CRLX101 particle size dependence on conjugate number.

FIGS. 13A and 13B depict line graphs of concentration-time curves and PK parameters for the formulated CDP-hexanoate-tofacitinib conjugate nanoparticles after intravenous (IV) (FIG. 13A) and subcutaneous (FIG. 13B) administrations, as compared to oral administration of unconjugated tofacitinib parent drug.

FIGS. 14A and 14B depict line graphs of a concentration-time curves and PK parameters for the formulated CDP-glycine-tofacitinib conjugate nanoparticles after intravenous (IV) (FIG. 14A) and subcutaneous (FIG. 14B) administrations, as compared to oral administration of unconjugated tofacitinib parent drug.

FIG. 15 depicts a line graph comparing paw volumes (as a percent of the initial paw volume at the time of arthritis induction on day 1) after administration of vehicle (), subcutaneous administration of dexamethasone (◯) at 1 mg/kg every other day for 7 cycles (q2d×7), oral administration of unconjugated tofacitinib parent drug (PO) at 10 mg/kg twice daily for 14 days (bid×14) (▾), and subcutaneous administration of formulated CDP-hexanoate-tofacitinib conjugate nanoparticles at 3 mg/kg every 7 days for 2 cycles (q7d×2) (▴) in a Lewis rat adjuvant-induced arthritis (AIA) model.

FIG. 16 depicts a line graph showing the effects of formulated CDP-hexanoate-tofacitinib conjugate nanoparticles on rat paw volume in the AIA model, as a percent of the initial paw volume at the time of arthritis induction on day 1 for vehicle control (), oral administration of unconjugated tofacitinib parent drug (PO) at 10 mg/kg twice daily for 14 days (bid×14) (▪), subcutaneous administration of formulated CDP-hexanoate-tofacitinib conjugate nanoparticles at 3 mg/kg every 7 days for 2 cycles (q7d×2) (▴), 1 mg/kg q7d×2 (▾), and 0.3 mg/kg q7d×2 (♦).

FIG. 17 depicts a line graph showing the effects of formulated CDP-hexanoate-tofacitinib conjugate nanoparticles on rat body weight in the AIA model (as a percent of the initial body weight at the time of arthritis induction on day 1) for vehicle control (), oral administration of unconjugated tofacitinib parent drug (PO) at 10 mg/kg twice daily for 14 days (bid×14) (▪), subcutaneous administration of formulated CDP-hexanoate-tofacitinib conjugate nanoparticles at 3 mg/kg every 7 days for 2 cycles (q7d×2) (▴), 1 mg/kg q7d×2 (▾), and 0.3 mg/kg q7d×2 (♦).

### DETAILED DESCRIPTION

The disclosure relates to novel compositions of therapeutic cyclodextrin-containing polymers (CDPs) conjugated to a JAK inhibitor, particles containing therapeutic cyclodextrin-containing polymers conjugated to a JAK inhibitor, compositions and mixtures comprising cyclodextrin-containing polymers, and methods of use thereof. In certain embodiments, these cyclodextrin-containing polymers improve JAK inhibitor stability and/or JAK inhibitor solubility, and/or reduce JAK inhibitor toxicity, and/or improve efficacy of the JAK inhibitor when used in vivo.

By selecting from a variety of linker groups used to link a JAK inhibitor to a CDP, the rate of JAK inhibitor release from the CDP can be attenuated for controlled delivery. The disclosure also relates to methods of treating subjects, e.g., humans, with a CDP-JAK inhibitor conjugate described herein.

More generally, the disclosure provides water-soluble, biocompatible polymer conjugates comprising a water-soluble, biocompatible cyclodextrin containing polymer covalently attached to a JAK inhibitor through attachments that are cleaved under biological conditions to release the JAK inhibitor.

Polymeric conjugates featured in the disclosure may be useful to improve solubility and/or stability of a bioactive/therapeutic agent, such as a JAK inhibitor, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into polymeric compounds of the disclosure.

An “effective amount” or “an amount effective” refers to an amount of the CDP-JAK inhibitor conjugate which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

“Pharmaceutically acceptable carrier or adjuvant,” as used herein, refers to a carrier or adjuvant that may be administered to a patient, together with a CDP-JAK inhibitor conjugate described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

As used herein, the term “prevent” or “preventing” as used in the context of the administration of an agent to a subject, refers to subjecting the subject to a regimen, e.g., the administration of a CDP-JAK inhibitor conjugate such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a CDP-JAK inhibitor conjugate such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

The term “hydrocarbyl” refers to a monovalent hydrocarbon radical comprised of carbon chains or rings to which hydrogen atoms are attached. The term includes alkyl, cycloalkyl, alkenyl, alkynyl and aryl groups, groups which have a mixture of saturated and unsaturated bonds, carbocyclic rings and includes combinations of such groups. Hydrocarbyl may refer to straight chain, branched-chain, cyclic structures or combinations thereof.

The term “hydrocarbylene” refers to a divalent hydrocarbyl radical.

### CDP-JAK Inhibitor Conjugates

Described herein are cyclodextrin containing polymer (“CDP”)-JAK inhibitor conjugates, wherein one or more JAK inhibitors are covalently attached to the CDP (e.g., either directly or through a linker). The CDP-JAK inhibitor conjugates include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952.

Accordingly, in one embodiment the CDP-JAK inhibitor conjugate is represented by Formula I:

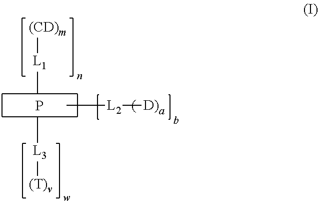

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a JAK inhibitor or a prodrug thereof;

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the JAK inhibitor moieties in the CDP-JAK inhibitor conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In some embodiments, one or more of the JAK inhibitor moieties in the CDP-JAK inhibitor conjugate can be replaced with another therapeutic agent, e.g. an agent that treats a cell, or cures, alleviates, relieves or improves one or more symptoms of a disease or disorder as described herein, e.g. a cancer, a cardiovascular disease, an autoimmune disease, an inflammatory disease, a metabolic disorder, a central nervous system disorder, or a neurological deficit.

In certain embodiments, P contains a plurality of cyclodextrin moieties within the polymer chain as opposed to the cyclodextrin moieties being grafted on to pendant groups off of the polymeric chain. Thus in certain embodiments, the polymer chain of formula I further comprises n′ units of U, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U is represented by one of the general formulae below:

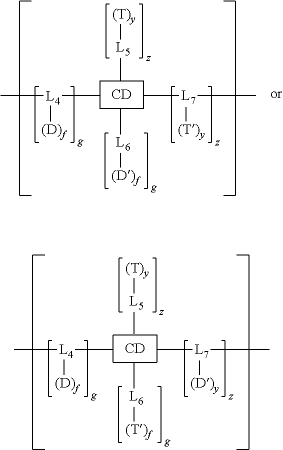

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different JAK inhibitor or prodrug forms thereof;

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. In some embodiments, at least 50% of the U units have at least one D or D′. In some embodiments, one or more of the JAK inhibitor moieties in the CDP-JAK inhibitor conjugate can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-JAK inhibitor conjugate is represented by Formula II:

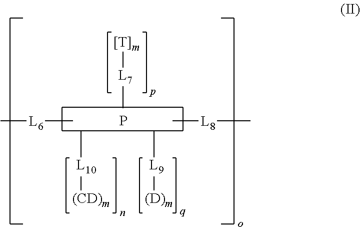

wherein

P represents a monomer unit of a polymer that comprises cyclodextrin moieties;

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a JAK inhibitor or a prodrug form thereof;

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the JAK inhibitor moieties in the CDP-JAK inhibitor conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-JAK inhibitor conjugate is represented either of the formulae below:

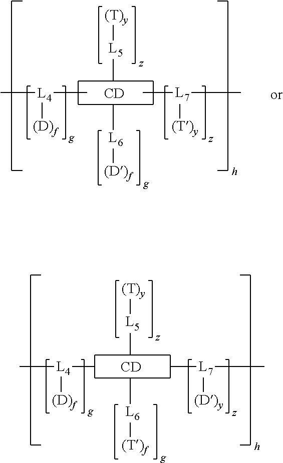

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different JAK inhibitor or prodrug thereof;

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

Preferably the polymer has a plurality of D or D′ moieties. In some embodiments, at least 50% of the polymer repeating units have at least one D or D′. In some embodiments, one or more of the JAK inhibitor moieties in the CDP-JAK inhibitor conjugate can be replaced with another therapeutic agent, e.g., another anticancer agent or anti-inflammatory agent.

In preferred embodiments, L4 and L7 represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6,15-crown-5,12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL.

Thus, in certain embodiments, the disclosure relates to novel compositions of therapeutic cyclodextrin-containing polymeric compounds designed for drug delivery of a JAK inhibitor. In certain embodiments, these CDPs improve drug stability and/or solubility, and/or reduce toxicity, and/or improve efficacy of the JAK inhibitor when used in vivo. Furthermore, by selecting from a variety of linker groups, and/or targeting ligands, the rate of JAK inhibitor release from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

Described herein are CDP-JAK inhibitor conjugates, wherein one or more JAK inhibitor is covalently attached to the CDP. The CDP can include linear or branched cyclodextrin-containing polymers and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, which are incorporated herein by reference in their entirety.

In some embodiments, the CDP-JAK inhibitor conjugate comprises a water soluble linear polymer conjugate comprising: cyclodextrin moieties; comonomers which do not contain cyclodextrin moieties (comonomers); and a plurality of JAK inhibitors; wherein the CDP-JAK inhibitor conjugate comprises at least four, five six, seven, eight, etc., cyclodextrin moieties and at least four, five six, seven, eight, or more, comonomers. In some embodiments, the JAK inhibitor is a JAK inhibitor described herein, for example, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543. The JAK inhibitor can be attached to the CDP via a functional group such as a hydroxyl group, or where appropriate, an amino group.

In some embodiments, one or more of the JAK inhibitor moieties in the CDP-JAK inhibitor conjugate can be replaced with another therapeutic agent, e.g., another anticancer agent or anti-inflammatory agent.

In some embodiments, the least four cyclodextrin moieties and at least four comonomers alternate in the CDP-JAK inhibitor conjugate. In some embodiments, said JAK inhibitors are cleaved from said CDP-JAK inhibitor conjugate under biological conditions to release the JAK inhibitor. In some embodiments, the cyclodextrin moieties comprise linkers to which JAK inhibitors are linked. In some embodiments, the JAK inhibitors are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a JAK inhibitor was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring. In some embodiments, the cyclodextrin moiety comprises an alpha, beta, or gamma cyclodextrin moiety. In some embodiments, at least about 50% of available positions on the CDP are reacted with a JAK inhibitor and/or a linker JAK inhibitor (e.g., at least about 55%, 60%, 65%, 70%, 75%, 80%, 85%, 90%, or 95%). In some embodiments, the JAK inhibitor is at least 5%, 10%, 15%, 20%, 25%, 30%, or 35% by weight of CDP-JAK inhibitor conjugate.

In some embodiments, the comonomer comprises polyethylene glycol of molecular weight of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)), the cyclodextrin moiety comprises beta-cyclodextrin, the theoretical maximum loading of the JAK inhibitor on the CDP-JAK inhibitor conjugate is about 25% by weight, and the JAK inhibitor is about 17-21% by weight of CDP-JAK inhibitor conjugate. In some embodiments, the JAK inhibitor is poorly soluble in water. In some embodiments, the solubility of the JAK inhibitor is <5 mg/ml at physiological pH. In some embodiments, the JAK inhibitor is a hydrophobic compound with a log P>0.4, >0.6, >0.8, >1, >2, >3, >4, or >5.

In some embodiments, the JAK inhibitor is attached to the CDP via a second compound.

In some embodiments, administration of the CDP-JAK inhibitor conjugate to a subject results in release of the JAK inhibitor over a period of at least 6 hours. In some embodiments, administration of the CDP-JAK inhibitor conjugate to a subject results in release of the JAK inhibitor over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-JAK inhibitor conjugate to a subject the rate of JAK inhibitor release is dependent primarily upon the rate of hydrolysis as opposed to enzymatic cleavage.

In some embodiments, the CDP-JAK inhibitor conjugate has a molecular weight of 10,000-500,000. In some embodiments, the cyclodextrin moieties make up at least about 2%, 5%, 10%, 20%, 30%, 50% or 80% of the CDP-JAK inhibitor conjugate by weight.

In some embodiments, the CDP-JAK inhibitor conjugate is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-JAK inhibitor conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-JAK inhibitor conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP-JAK inhibitor conjugate is a polymer having attached thereto a plurality of D moieties of the following formula:

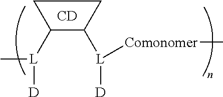

wherein each L is independently a linker, and each D is independently a JAK inhibitor, a prodrug derivative thereof, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one JAK inhibitor and in some embodiments, at least two JAK inhibitor moieties. In some embodiments, the molecular weight of the comonomer is from about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)).

In some embodiments, the JAK inhibitor is a JAK inhibitor described herein, for example, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543. The JAK inhibitor can be attached to the CDP via a functional group such as a hydroxyl group, or where appropriate, an amino group. In some embodiments, one or more of the JAK inhibitor moieties in the CDP-JAK inhibitor conjugate can be replaced with another therapeutic agent, e.g., another anticancer agent or anti-inflammatory agent.

In some embodiments, the CDP-JAK inhibitor conjugate is a polymer having attached thereto a plurality of D moieties of the following formula:

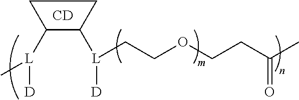

wherein each L is independently a linker, and each D is independently a JAK inhibitor, a prodrug derivative thereof, or absent, provided that the polymer comprises at least one JAK inhibitor and in some embodiments, at least two JAK inhibitor moieties (e.g., at least 3, 4, 5, 6, 7, 8, 9, 10, 11, 12, 13, 14, 15, 16, 17, 18, 19, 20 or more); and wherein the group

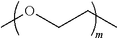

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, the JAK inhibitor is a JAK inhibitor described herein, for example, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543. The JAK inhibitor can be attached to the CDP via a functional group such as an amino group, or where appropriate, a hydroxyl group. In some embodiments, one or more of the JAK inhibitor moieties in the CDP-JAK inhibitor conjugate can be replaced with another therapeutic agent, e.g., another anticancer agent or anti-inflammatory agent.

In some embodiments, less than all of the L moieties are attached to D moieties, meaning in some embodiments, at least one D is absent. In some embodiments, the loading of the D moieties on the CDP-JAK inhibitor conjugate is from about 1 to about 50% by weight of the polymer (e.g., from about 1 to about 25%, from about 5 to about 20% or from about 5 to about 15% by weight of the polymer). In some embodiments, each L independently comprises an amino acid or a derivative thereof. In some embodiments, each L independently comprises a plurality of amino acids or derivatives thereof. In some embodiments, each L is independently a dipeptide or derivative thereof.

In some embodiments, the CDP-JAK inhibitor conjugate is a polymer having attached thereto a plurality of L-D moieties of the following formula:

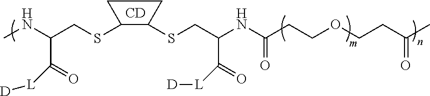

wherein each L is independently a linker or absent and each D is independently a JAK inhibitor, a prodrug derivative thereof, or absent and wherein the group

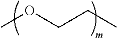

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20, provided that the polymer comprises at least one JAK inhibitor and in some embodiments, at least two JAK inhibitor moieties (e.g., at least 3, 4, 5, 6, 7, 8, 9, 10, 11, 12, 13, 14, 15, 16, 17, 18, 19, 20 or more).

In some embodiments, L comprises a self-cyclizing moiety. In some embodiments, L comprises both a self-cyclizing moiety and a selectivity-determining moiety.

In some embodiments, the CDP-JAK inhibitor conjugate is a polymer of the following formula:

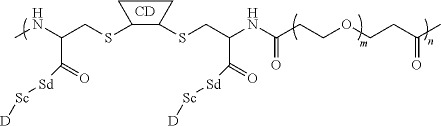

wherein each Sd is independently a selectivity-determining moiety or absent, each Sc is independently a self-cyclizing moiety or absent, and each D is independently a JAK inhibitor, e.g., a JAK inhibitor described herein, a prodrug derivative thereof, or absent and wherein the group

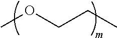

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20, provided that the polymer comprises at least one JAK inhibitor, e.g., a JAK inhibitor described herein, or a prodrug derivative thereof, and in some embodiments, at least two JAK inhibitors, e.g., JAK inhibitors described herein, or a prodrug derivatives thereof (e.g., at least 3, 4, 5, 6, 7, 8, 9, 10, 11, 12, 13, 14, 15, 16, 17, 18, 19, 20 or more).

In some embodiments, the JAK inhibitor is a JAK inhibitor described herein, for example, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543.

In some embodiments, less than all of the C(═O) moieties are attached to L-D moieties, meaning in some embodiments, at least one L and/or D is absent. In some embodiments, the loading of the L, D and/or L-D moieties on the CDP-JAK inhibitorconjugate is from about 1 to about 50% (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the JAK inhibitor moieties in the CDP-JAK inhibitorconjugate can be replaced with another therapeutic agent, e.g., another anticancer agent or anti-inflammatory agent.

In some embodiments, the CDP-JAK inhibitor conjugate is a polymer having the following formula:

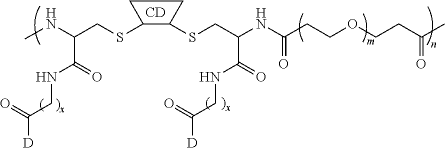

wherein each x is independently 1, 2, 3, 4, or 6, each D is independently a JAK inhibitor, e.g., a JAK inhibitor described herein, a prodrug derivative thereof, or absent and wherein the group

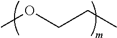

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, less than all of the C(═O) moieties are attached to

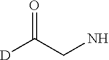

moieties, meaning in some embodiments,

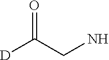

is absent, provided that the polymer comprises at least one JAK inhibitor and in some embodiments, at least two JAK inhibitor moieties (e.g., at least 3, 4, 5, 6, 7, 8, 9, 10, 11, 12, 13, 14, 15, 16, 17, 18, 19, 20 or more). In some embodiments, the loading of the

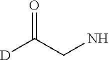

moieties on the CDP-JAK inhibitor conjugate is from about 1 to about 50% (e.g., from about 1 to about 25%, from about 5 to about 25% or from about 15 to about 15%).

In some embodiments, the CDP-JAK inhibitor conjugate is a polymer having the following formula:

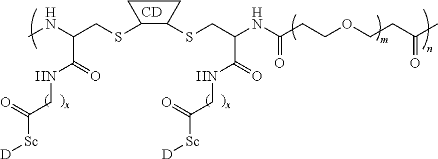

wherein each x is independently 1, 2, 3, 4, or 5 and each Sc is independently a self-cyclizing moiety.

In some embodiments, the JAK inhibitor is a JAK inhibitor described herein, for example, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543.

In some embodiments, one or more of the JAK inhibitor moieties in the CDP-JAK inhibitor conjugate can be replaced with another therapeutic agent, e.g., another anticancer agent or anti-inflammatory agent.

In some embodiments, the CDP-JAK inhibitor conjugate will contain a JAK inhibitor and at least one additional therapeutic agent. For instance, a JAK inhibitor and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

### Cyclodextrins

In certain embodiments, the cyclodextrin moieties make up at least about 2%, 5% or 10% by weight, up to 20%, 30%, 50% or even 80% of the CDP by weight. In certain embodiments, the JAK inhibitors, or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, Mₙ varies between about 12,000 and 65,000 daltons. In certain other embodiments, Mₙ varies between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta ((β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

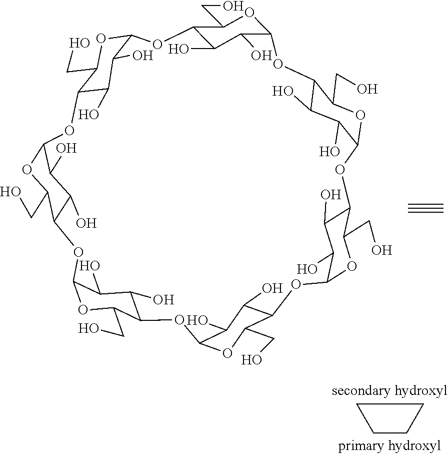

The side on which the secondary hydroxyl groups are located has a wider diameter than the side on which the primary hydroxyl groups are located. The disclosure contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-JAK inhibitorconjugate is oxidized. In certain embodiments, the cyclodextrin moieties of P alternate with linker moieties in the polymer chain.

### Comonomers

In addition to a cyclodextrin moiety, the CDP can also include a comonomer, for example, a comonomer described herein. In some embodiments, a comonomer of the CDP-JAK inhibitor conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-JAK inhibitor conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, a comonomer can be and/or can comprise a linker such as a linker described herein.

### Linkers/Tethers

The CDPs described herein can include one or more linkers. In some embodiments, a linker, such as a linker described herein, can link a cyclodextrin moiety to a comonomer. In some embodiments, a linker can link a JAK inhibitor to a CDP. In some embodiments, for example, when referring to a linker that links a JAK inhibitor to the CDP, the linker can be referred to as a tether.

In certain embodiments, a plurality of the linker moieties are attached to a JAK inhibitor or prodrug thereof and are cleaved under biological conditions.

Described herein are CDP-JAK inhibitor conjugates that comprise a CDP covalently attached to JAK inhibitors through attachments that are cleaved under biological conditions to release the JAK inhibitor. In certain embodiments, a CDP-JAK inhibitor conjugate comprises a JAK inhibitor covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the JAK inhibitor.

In some embodiments, such JAK inhibitors are covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-JAK inhibitor conjugatecomprises a JAK inhibitor covalently attached to the CDP through a tether, wherein the tether comprises a self-cyclizing moiety. In some embodiments, the tether further comprises a selectivity-determining moiety. Thus, one aspect of the disclosure relates to a polymer conjugate comprising a therapeutic agent covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under hydrolysis conditions, enzymatic conditions, acidic conditions or basic conditions.

In certain embodiments, the disclosure contemplates any combination of the foregoing. Those skilled in the art will recognize that, for example, any CDP of the disclosure in combination with any linker (e.g., a linker described herein such as a self-cyclizing moiety, any selectivity-determining moiety, and/or any JAK inhibitor) are within the scope of the disclosure.

In certain embodiments, the selectivity-determining moiety is selected such that the bond is cleaved under acidic conditions.

In certain embodiments, the selectivity-determining moiety comprises an ester moiety that is cleaved by hydrolysis conditions.

In certain embodiments where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

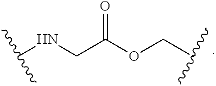

In certain embodiments where the selectivity-determining moiety is selected such that the bond is cleaved under basic conditions, the selectivity-determining moiety is an aminoalkylcarbonyloxy moiety. In certain embodiments, the selectivity-determining moiety has a structure

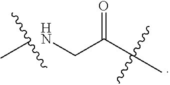

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA and GFLG, preferably GFLG.

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by Formula A:

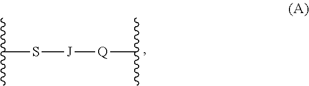

wherein
S is a sulfur atom that is part of a disulfide bond;
J is optionally substituted hydrocarbyl; and
Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

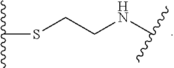 [SMILES: *NCCS*]

In certain embodiments, the selectivity-determining moiety is represented by Formula B:

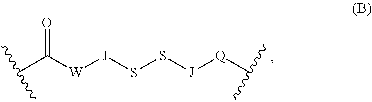

wherein
W is either a direct bond or selected from lower alkyl, NR¹⁴, S, O;
S is sulfur;
J, independently and for each occurrence, is hydrocarbyl or polyethylene glycol;
Q is O or NR¹³, wherein R¹³ is hydrogen or alkyl; and
R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{X}R^{X}, —CO₂OR^{x}, —C(O)—NR^{x}R^{x}, —C(O)—R^{x}, —NR^{x}—C(O)—R^{x}, —NR^{x}SO₂R^{x}, —SR^{X}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{X}, —NR^{x}—C(O)—R^{x}, —NR^{x}SO₂R^{x}, —SR^{X}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

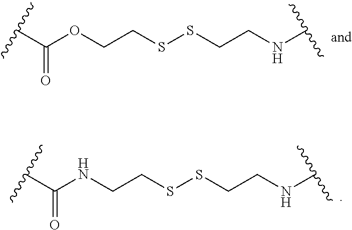 [SMILES: *NCCSSCCOC(*)=O ; *NCCSSCCNC(*)=O]

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

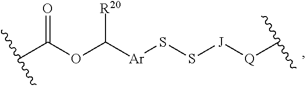

wherein
Ar is a substituted or unsubstituted benzo ring;
J is optionally substituted hydrocarbyl; and

### Q is O or NR¹³,

wherein R¹³ is hydrogen or alkyl.

In certain embodiments, Ar is unsubstituted. In certain embodiments, Ar is a 1,2-benzo ring. For example, suitable moieties within Formula B include

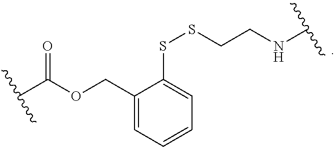 [SMILES: *NCCSSc1ccccc1COC(*)=O]

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone. In certain embodiments, the self-cyclizing moiety cyclizes to form a five-membered ring comprising at least one oxygen atom, preferably two. In certain such embodiments, the self-cyclizing moiety cyclizes to form a 1,3-dioxolan-2-one.

In certain embodiments, the self-cyclizing moiety comprises a structure

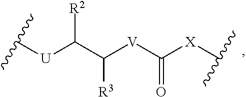

wherein
U is selected from O, NR¹ and S;
X is a heteroatom, e.g., O, S, or N, of the JAK inhibitor, e.g., a portion of the JAK inhibitor described herein;
V is selected from O, S and NR⁴, preferably O or NR⁴;
R² and R³ are independently selected from hydrogen, alkyl, and alkoxy; or R² and R³ together with the carbon atoms to which they are attached form a ring; and
R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl.

In certain embodiments, X is a nitrogen of a heterocycloalkyl or heteroaryl moiety, e.g., imidazolyl, pyrrolyl, pyrazolyl, triazolyl, pyrrolidinyl, 2-pyrroline, 3-pyrroline, 2-imidazolidinyl, 2-pyrazolinyl, pyrazolidinyl, 1,2,3-triazolyl, piperidinyl, morpholinyl, thiomorpholinyl, piperazinyl, indolyl, isoindolyl, indolinyl, 1H-indazolyl, benzimidazolyl, purinyl, pyrrolopyrimidinyl, carbazolyl, phenothiazinyl, phenoxazinyl, that is a portion of the JAK inhibitor. In certain embodiments, X is the nitrogen of a pyrrolyl, pyrrolopyrimidinyl, pyrazolyl, imidazolyl moiety of a portion of the JAK inhibitor. In certain embodiments, X is:

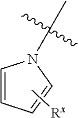

wherein R^{x} depicts the portion of the structure of the JAK inhibitor not depicted above. For example, the following JAK inhibitors are attached to the self-cyclizing moiety as depicted below:

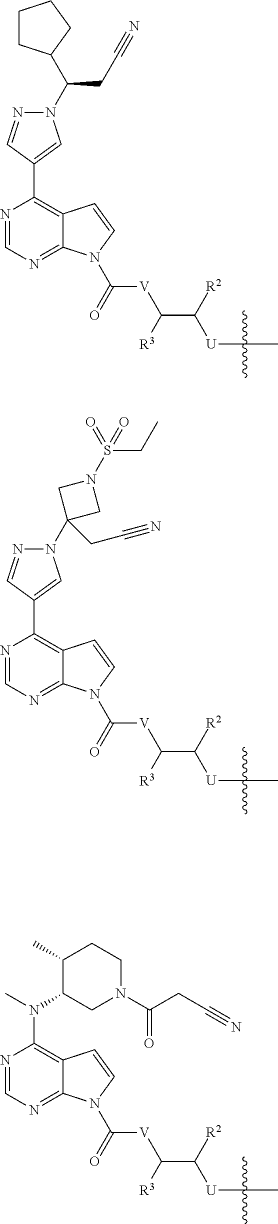

In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together with the carbon atoms to which they are attached form a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments V is —NH— or O. In certain embodiments U is —NH— or O. In certain embodiments at least one of V or U is O. In certain embodiments, U is bonded to the CDP or a selectivity-determining moiety.

In certain embodiments, the self-cyclizing moiety is selected from

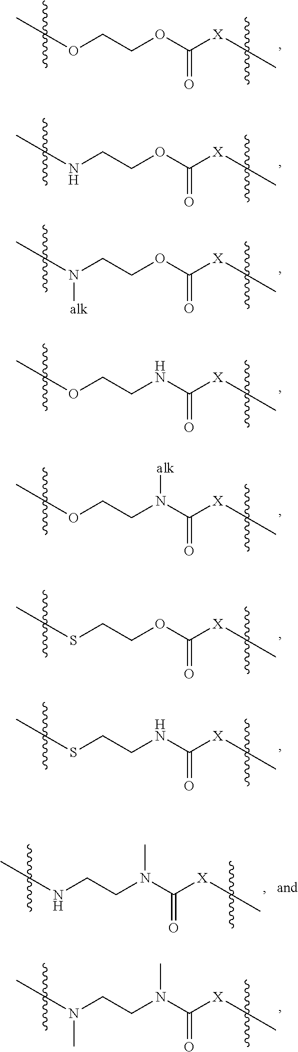

wherein “alk” is a C₁₋₆ alkyl group; and “X” denotes a portion of the JAK inhibitor.

In certain embodiments, the selectivity-determining moiety can connect to the self-cyclizing moiety through carbonyl-heteroatom bonds, e.g., amide, carbamate, carbonate, ester, thioester, and urea bonds. In some embodiments the selectivity-determining moiety comprises an ester.

In certain embodiments, a JAK inhibitor is covalently attached to a polymer through a linker, wherein the linker comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH maybe be a self-cyclizing moiety, and ABC may be selected such that enzyme Y cleaves between C and D. Once cleavage of the bond between C and D progresses to a certain point, D will cyclize onto H, thereby releasing therapeutic agent, e.g., JAK inhibitor, X, or a prodrug thereof.

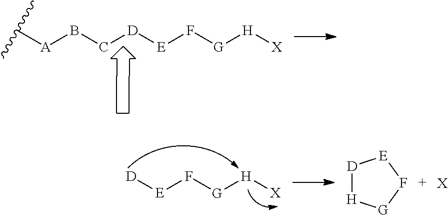

In certain embodiments, the JAK inhibitor is covalently attached to a CDP through a linker, wherein the linker comprises a selectivity-determining moiety and a self-cyclizing moiety, which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent, e.g., JAK inhibitor, e.g., JAK inhibitor described herein. As an illustration, the selectivity-determining moiety can be a moiety of the formula:

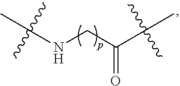

wherein p is 1 to 6, e.g., 1, 2, 3, 4, 5, 6. In certain embodiments p is 1. In certain embodiments p is 6.

As an illustration, the self-cyclizing moiety can be a moiety of the formula:

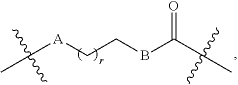

wherein A and B are heteroatoms independently selected from O, N, or S, and r is 1, 2, or 3, e.g., 1. In some embodiments, at least one of A and B is O. In certain embodiments A and B are both O. As an illustration, the linker, can be a linker comprising a selectivity-determining moiety and a self-cyclizing moiety, which are covalently attached to one another, and has the following formula:

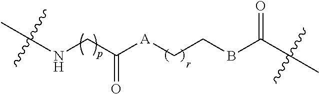

wherein the variables A, B, p and r are as described above.

In certain embodiments, the JAK inhibitor (“X”) is covalently attached to a CDP through a linker, as described above. In certain embodiments, the self-cyclizing moiety is selected such that after cleavage of the bond between the selectivity-determining moiety and the self-cyclizing moiety, e.g., the bond between “A” and the carbonyl group, cyclization of the self-cyclizing moiety occurs, thereby releasing the JAK inhibitor (“X”). In the embodiment shown above the carboxyl moiety of the CDP is attached to the linker, through the nitrogen of the selectivity-determining moiety, e.g., via an amide bond between the carboxyl moiety of the CDP and the nitrogen of the selectivity-determining moiety. The scheme depicting the cleavage of the bond between the selectivity-determining moiety and the self-cyclizing moiety, cyclization of the self-cyclizing moiety, and release of the JAK inhibitor is shown in the below scheme.

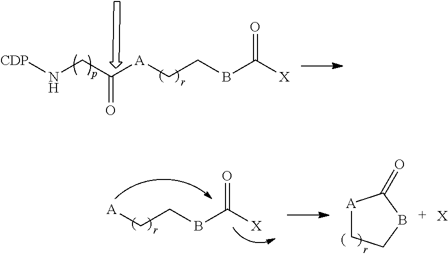

In certain embodiments, X is

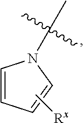

wherein R^{x} depicts the portion of the structure of the JAK inhibitor not depicted. The scheme depicting the cleavage of the bond between the selectivity-determining moiety and the self-cyclizing moiety, cyclization of the self-cyclizing moiety, and release of the JAK inhibitor, e.g., a JAK inhibitor having a pyrrole moiety, is shown in the below scheme.

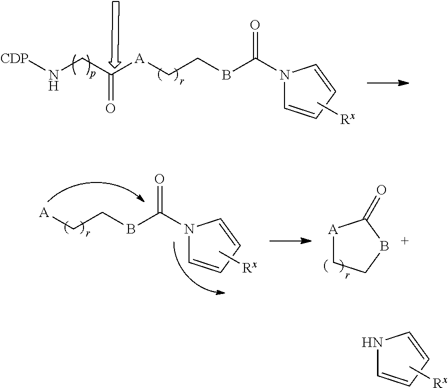

For example, the following JAK inhibitors can be used with a tether, e.g., linker, comprising a selectivity-determining moiety and a self-cyclizing moiety as described above. In certain embodiments, the JAK inhibitor is Tofacitinib and is released from the linker as shown in the below scheme.

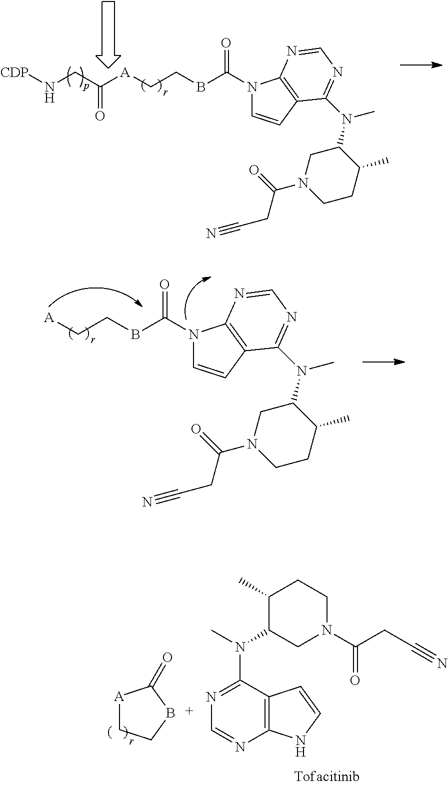

In certain embodiments, the JAK inhibitor is Ruxolitinib and is released from the linker as shown in the below scheme.

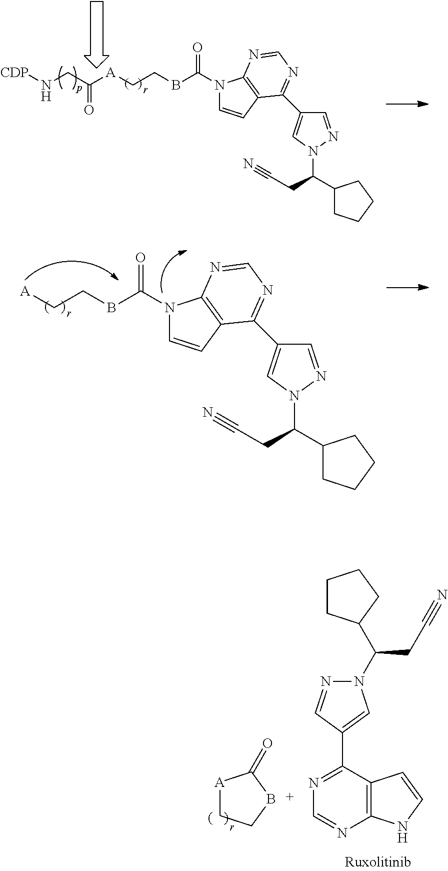

In certain embodiments, the JAK inhibitor is Baricitinib and is released from the linker as shown in the below scheme.

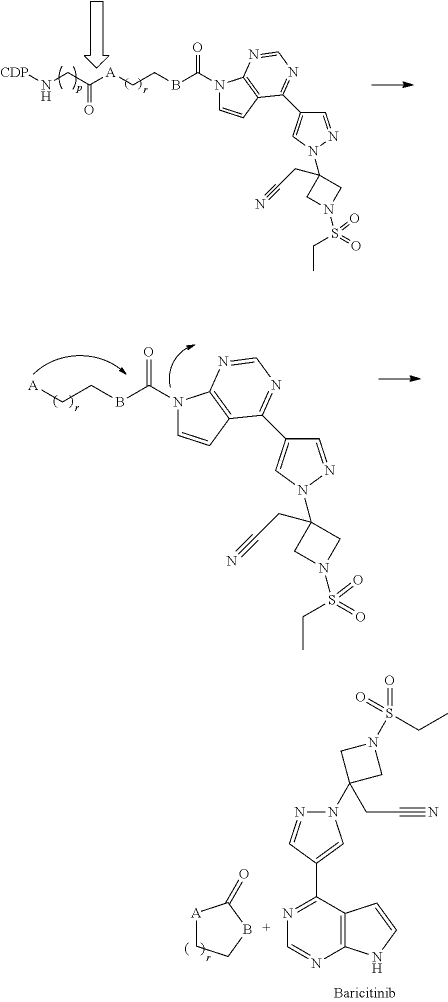

In certain embodiments, X is —O—R^{x}, wherein R^{x} depicts the portion of the structure of the JAK inhibitor not depicted. The scheme depicting the cleavage of the bond between the selectivity-determining moiety and the self-cyclizing moiety, cyclization of the self-cyclizing moiety, and release of the JAK inhibitor, e.g., a JAK inhibitor having a hydroxyl moiety, is shown in the below scheme.

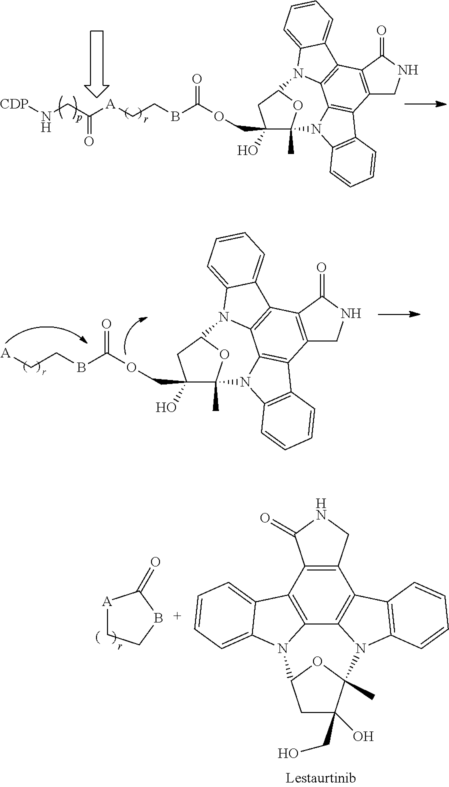

In certain embodiments, JAK inhibitor “X” may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker can comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments the linker group(s) of the disclosure represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, a linker group, e.g., between a JAK inhibitor and the CDP, comprises a self-cyclizing moiety. In certain embodiments, a linker group, e.g., between a JAK inhibitor and the CDP, comprises a selectivity-determining moiety.

In certain embodiments as disclosed herein, a linker group, e.g., between a JAK inhibitor and the CDP, comprises a self-cyclizing moiety and a selectivity-determining moiety.

In certain embodiments as disclosed herein, the JAK inhibitor or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to JAK inhibitors or prodrugs thereof that are cleaved under biological conditions.

In certain embodiments, the linker group comprises an amino acid or peptide, or derivative thereof (e.g., a glycine or cysteine).

In certain embodiments as disclosed herein, the linker is connected to the JAK inhibitor through a hydroxyl group (e.g., forming an ester bond). In certain embodiments as disclosed herein, the linker is connected to the JAK inhibitor through an amino group (e.g., forming an amide bond).

In certain embodiments, the linker group that connects to the JAK inhibitor may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

In one embodiment, the linker used to link the JAK inhibitor to a CDP controls the rate of JAK inhibitor release from the CDP. For example, the linker can be a linker which in the PBS protocol described herein, releases within 24 hours as free JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), 70%, 75%, 80%, 85%, 86%, 87%, 88%, 89%, 90%, 91%, 92%, 93%, 94%, 95%, 96%, 97%, 98%, 99% or all of the JAK inhibitor in the CDP-JAK inhibitor conjugate initially present in the assay. In some embodiments, in the PBS protocol described herein, the linker releases 71±10% of the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, within 24 hours, wherein 71 is the % of JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) released from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, at 24 hours by a reference structure, e.g., a JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) coupled via 2-(2-(2-aminoethoxy)ethoxy)acetic acetate (i.e., aminoethoxyethoxy) to the same CDP in the PBS protocol described herein.

In other embodiments, the linker releases 88±10% of the JAK inhibitor from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, within 24 hours, wherein 88 is the % of JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), released from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, at 24 hours by a reference structure, e.g., JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), coupled via glycine to the same CDP in the PBS protocol described herein or the linker releases 95±5% of the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, within 24 hours, wherein 95 is the % of JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), released from the CDP-JAK inhibitor conjugate, e.g., CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, at 24 hours by a reference structure, e.g., JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) coupled via alanine glycolate to the same CDP in the PBS protocol described herein. Such linkers include linkers which are released by hydrolysis of an ester bond, which hydrolysis releases JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) conjugated to CDP from CDP. In one embodiment, the linker is selected from glycine, alanine glycolate and 2-(2-(2-aminoethoxy)ethoxy)acetic acetate (i.e., aminoethoxyethoxy). In one embodiment, the linker used to link JAK inhibitor to a CDP attaches to the JAK inhibitor via an ester linkage and the CDP via an amide linkage. In some preferred embodiments, the linker includes a heteroatom attached to the carbon positioned alpha to the carbonyl carbon that forms the ester linkage with the JAK inhibitor.

### Benzyl Elimination Chemistry

In some embodiments, the linker is attached to the JAK inhibitor through a nitrogen that is part of the JAK inhibitor. In certain such embodiments, the linker can comprise a benzyl elimination linker, e.g., a benzyl moiety that eliminates after cleavage, e.g., hydrolysis or reduction, of the selectivity-determining moiety, to release the therapeutic agent, e.g., the JAK inhibitor, e.g., the JAK inhibitor as described herein. In certain embodiments, the linker comprising a benzyl elimination linker has the structure of the formula:

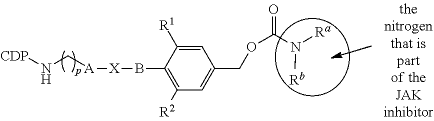

wherein

### A is O, S, or —CR¹CR²—;

### B is O or S;

X is —C(O)— or a bond;
R¹ and R² are H or C₁₋₆ alkyl;
R^{a} is H or C₁₋₆ alkyl; R^{b} is part of the JAK inhibitor; or R^{a} and R^{b} together with the nitrogen to which they are attached is part of the JAK inhibitor; and
p is 1, 2, 3, 4, or 5.

In some embodiments, —NR^{a}R^{b} represents a pyrrole moiety that is part of the JAK inhibitor. In certain such embodiments, the JAK inhibitor is Tofacitinib, Ruxolitinib, or Baricitinib.

In some embodiments, —NR^{a}R^{b} represents a piperidine moiety that is part of the JAK inhibitor, e.g., NVP-BSK805.

In some embodiments, —NR^{a}R^{b} represents a heteroaromatic amine moiety that is part of the JAK inhibitor, e.g., INCB16562, XL019, Pacritinib, CYT387, AZD1480, TG101348, CEP33779, BMS911543, or VX-509.

In some embodiments, A is O, B is O, X is —C(O)—, and —NR^{a}R^{b} represents a pyrrole moiety that is part of the JAK inhibitor. In certain such embodiments, the JAK inhibitor is Tofacitinib, Ruxolitinib, or Baricitinib.

In some embodiments, A is —CR¹CR²—, B is O, X is —C(O)—, and —NR^{a}R^{b} represents a pyrrole moiety that is part of the JAK inhibitor. In certain such embodiments, the JAK inhibitor is Tofacitinib, Ruxolitinib, or Baricitinib.

In some embodiments, A is —CR¹CR²—, B is S, X is a bond, and —NR^{a}R^{b} represents a pyrrole moiety that is part of the JAK inhibitor. In certain such embodiments, the JAK inhibitor is Tofacitinib, Ruxolitinib, or Baricitinib.

In some embodiments, p is 1. In some embodiments, p is 3. In some embodiments, p is or 5.

In some embodiments, the linker comprises a selectivity-determining moiety, e.g., a carbonate, ester, or disulfide moiety. In some embodiments, the linker comprising a benzyl elimination linker has one of the following structures:

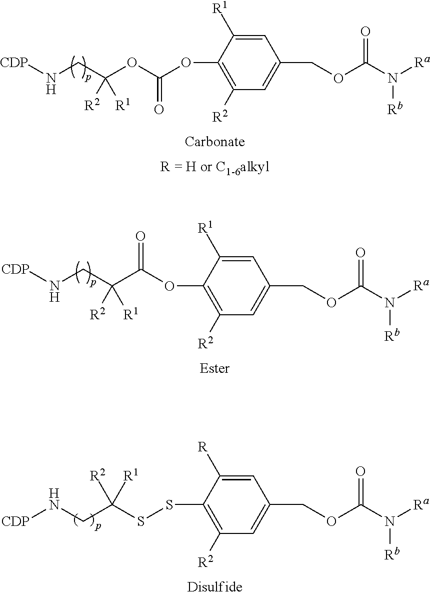

In certain such embodiments the linker can comprise a selectivity-determining moiety comprising, e.g., a carbonate, an ester or a disulfide moiety, and the pyrrole moiety is part of the JAK inhibitor, as shown in the scheme below.

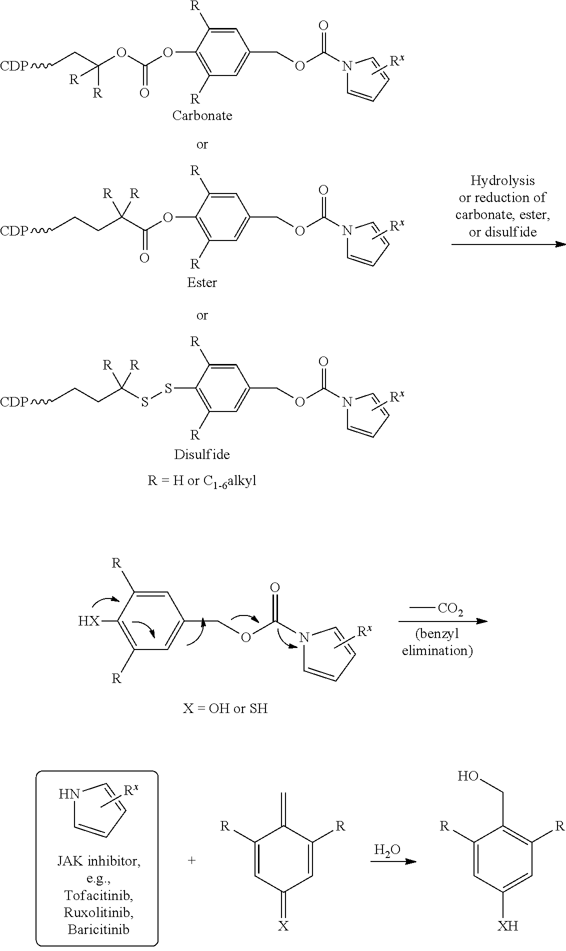

In certain embodiments, a JAK inhibitor, e.g., a JAK inhibitor described herein, can be linked to the CDP via a benzyl elimination linker comprising a selectivity-determining moiety comprising a carbonate moiety as shown in the scheme below.

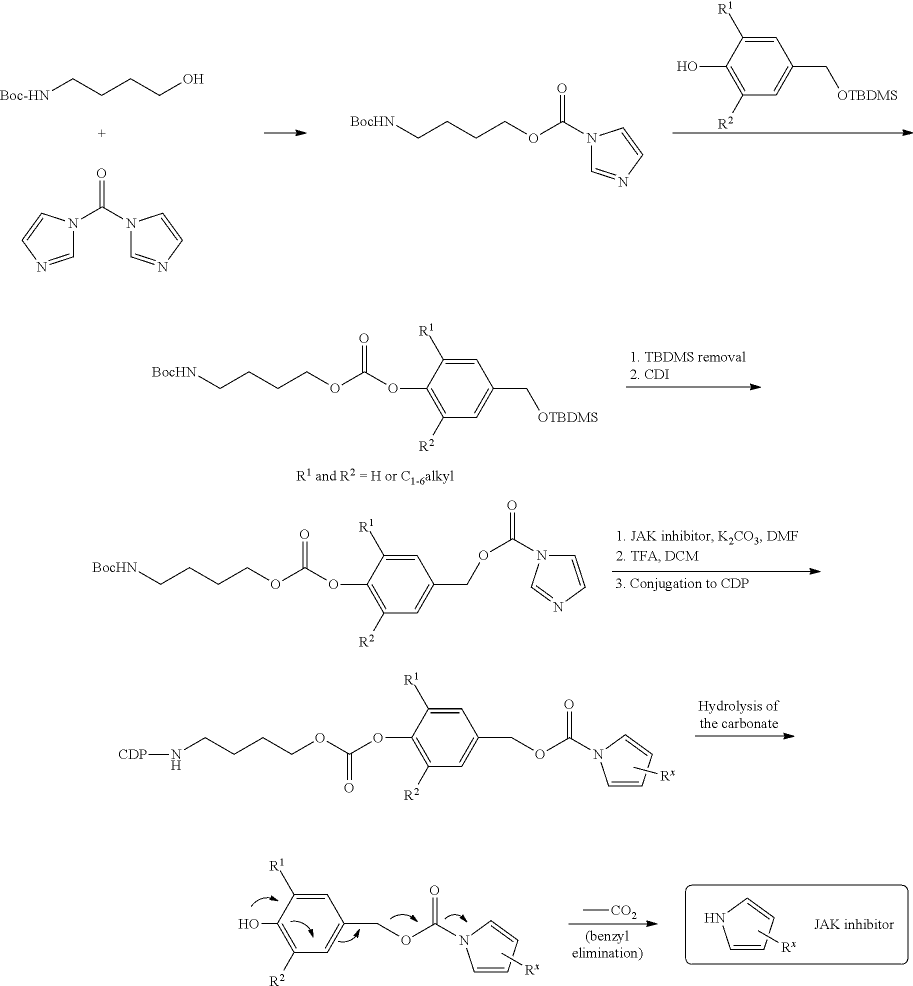

In certain such embodiments, a JAK inhibitor, e.g., tofacitinib, can be linked to the CDP via a benzyl elimination linker comprising a selectivity-determining moiety comprising a carbonate moiety as shown in the scheme below.

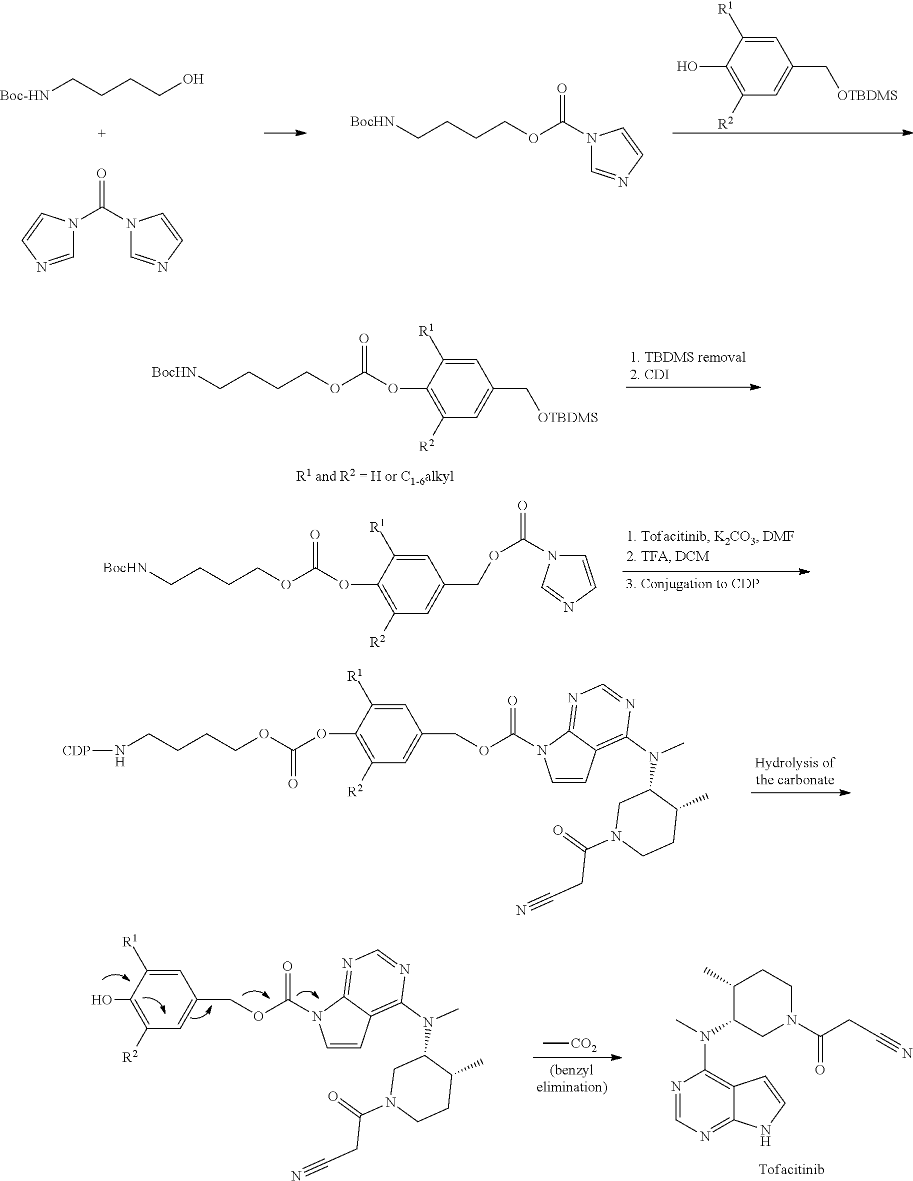

In certain embodiments, a JAK inhibitor, e.g., a JAK inhibitor described herein, can be linked to the CDP via a benzyl elimination linker comprising a selectivity-determining moiety comprising a ester moiety as shown in the scheme below.

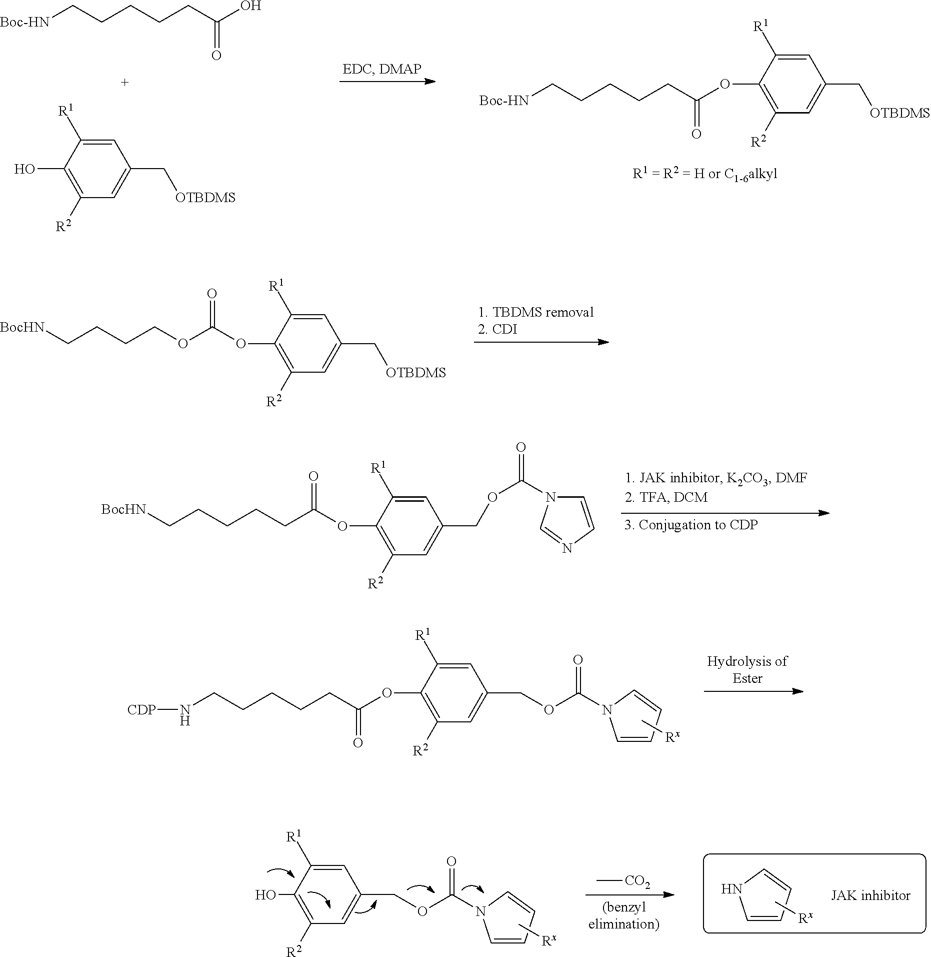

In certain embodiments, a JAK inhibitor, e.g., tofacitinib, can be linked to the CDP via a benzyl elimination linker comprising a selectivity-determining moiety comprising a ester moiety as shown in the scheme below.

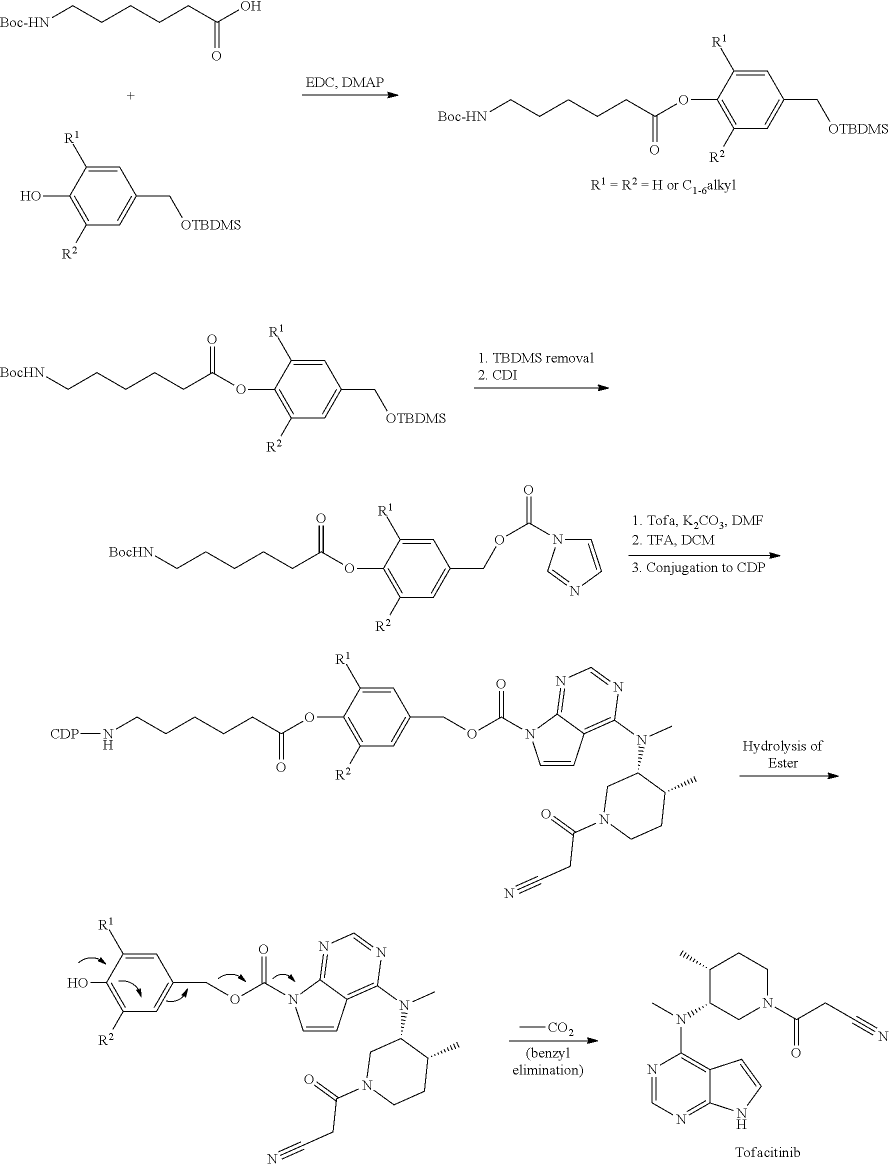

In certain embodiments, a JAK inhibitor, e.g., a JAK inhibitor described herein, can be linked to the CDP via a benzyl elimination linker comprising a selectivity-determining moiety comprising a disulfide moiety as shown in the scheme below.

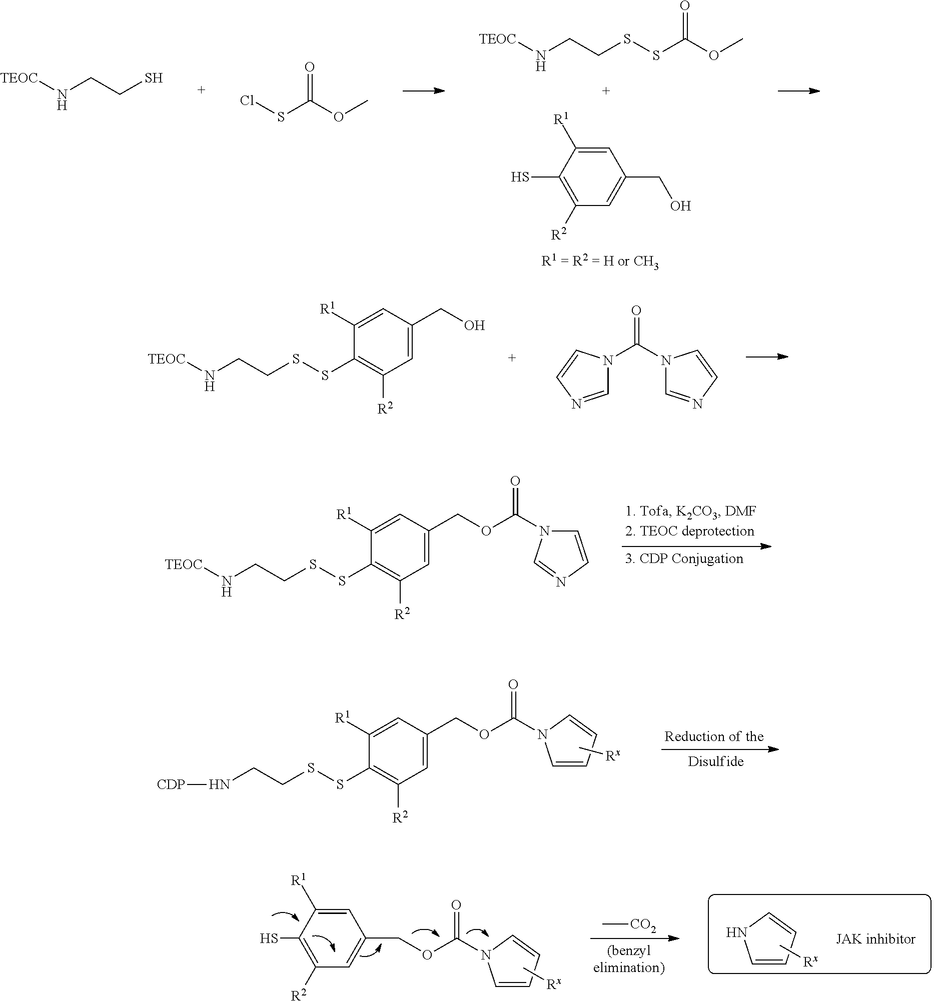

In certain embodiments, a JAK inhibitor, e.g., tofacitinib, can be linked to the CDP via a benzyl elimination linker comprising a selectivity-determining moiety comprising a disulfide moiety as shown in the scheme below.

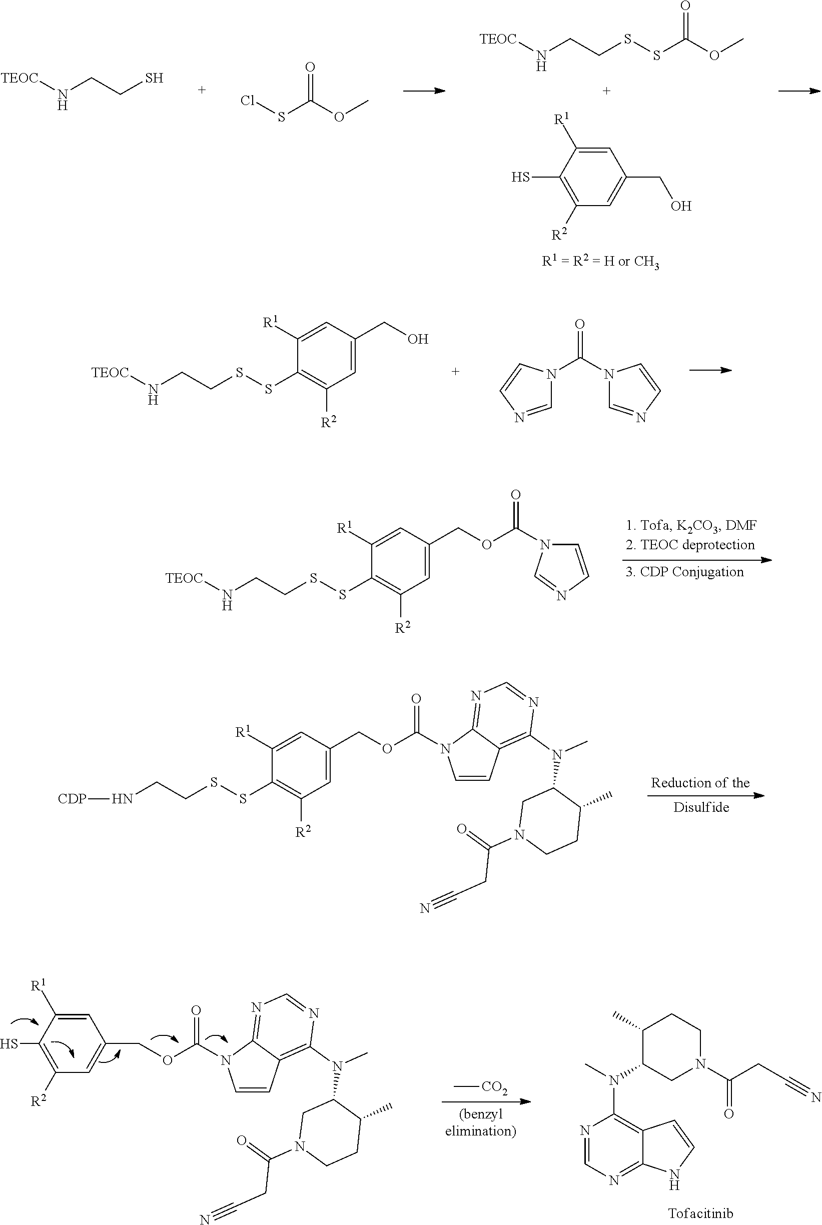

In certain embodiments, the JAK inhibitor is linked to the CDP through the hydroxyl moiety, e.g., primary or secondary hydroxyl moiety, of the JAK inhibitor, e.g., Lestaurtinib.

In certain such embodiments, the linker used to link the JAK inhibitor, e.g., Lestaurtinib, to a CDP has the following formula

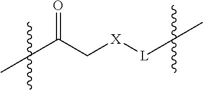

wherein

X is O, NH, or Nalkyl; and

L is an alkylenyl or heteroalkylenyl chain, wherein one or more of the carbons of the alkylenyl or heteroalkylenyl are optionally substituted (e.g., with an oxo moiety), or wherein L is absent; or

L has the formula

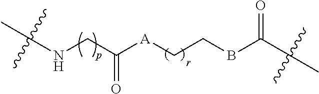

wherein the variables A, B, p and r are as described above;

wherein the carbonyl portion of the linker attaches to the JAK inhibitor, e.g., Lestaurtinib, to form an ester linkage; and

wherein the X-L portion of the linker attaches to the CDP to form an amide bond.

In one embodiment, X is NH. In one embodiment, X is NH and L is absent.

In one embodiment, X is O. In one embodiment, X is O and L is an alkylenyl or heteroalkylenyl chain, wherein one or more of the carbons of the alkylenyl or heteroalkylenyl are optionally substituted (e.g., with an oxo moiety). In one embodiment, L is —C(O)CH₂CH₂NH—.

In some embodiments, the linker can be a linker which in the B16.F10 cell assay described herein, releases free JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), of the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), in the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein, such that the IC₅₀ of the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), is less than 25 nM, 20 nM, 15 nM, 10 nM, 5 nM, 4 nM, 3 nM, 2 nM, 1 nM, 0.5 nM or 0.1 nM. In some embodiments, in the B16.F10 assay described herein, the linker releases the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543), from the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described herein such that the IC₅₀ of the JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) is less than 5 nM, 4 nM, 3 nM, 2 nM, 1 nM, 0.5 nM. Such linkers include linkers that are released by hydrolysis of an ester bond, which hydrolysis releases docetaxel conjugated to CDP from CDP and linkers which are released by chemical or enzymatic cleavage of a disulfide bond, whereby enzymatic cleavage releases JAK inhibitor (e.g., ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543) conjugated to CDP from CDP. In one embodiment, the linker is selected from glycine, hexanoate, alanine glycolate and dithiolethyloxy-carbonate.

In certain embodiments, the disclosure contemplates a CDP, wherein a plurality of JAK inhibitors are covalently attached to the polymer through attachments that are cleaved under biological conditions to release the JAK inhibitors as discussed above, wherein administration of the polymer to a subject results in release of the therapeutic agent over a period of at least 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month.

In some embodiments, the conjugation of the JAK inhibitor to the CDP improves the aqueous solubility of the JAK inhibitor and hence the bioavailability. Accordingly, in one embodiment of the disclosure, the JAK inhibitor has a log P>0.4, >0.6, >0.8, >1, >2, >3, >4, or even >5.

The CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described hereinpreferably have a molecular weight in the range of 10,000 to 500,000; 30,000 to 200,000; or even 70,000 to 150,000 amu.

In certain embodiments, the disclosure contemplates attenuating the rate of release of the JAK inhibitor by introducing various tether and/or linking groups between the therapeutic agent and the polymer. Thus, in certain embodiments, the CDP-JAK inhibitor conjugate, e.g., a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate or a CDP-BMS 911543 conjugate described hereinare compositions for controlled delivery of the JAK inhibitor.

### JAK Inhibitors

Protein kinases can be categorized as receptor type and non-receptor type. Receptor tyrosine kinases (RTKs) have an extracellular portion, a transmembrane domain, and an intracellular portion, while non-receptor tyrosine kinases are entirely intracellular. The Janus kinase family of protein tyrosine kinases (JAKs) belong to the non-receptor type of tyrosine kinases and include family members: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2).

The pathway involving JAKs and Signal Transducers and Activators of Transcription (STATs) is engaged in the signaling of a wide range of cytokines. Cytokines are low-molecular weight polypeptides or glycoproteins that stimulate biological responses in virtually all cell types. Generally, cytokine receptors do not have intrinsic tyrosine kinase activity, and thus require receptor-associated kinases to propagate a phosphorylation cascade. JAKs fulfill this function. Cytokines bind to their receptors, causing receptor dimerization, and this enables JAKs to phosphorylate each other as well as specific tyrosine motifs within the cytokine receptors. STATs that recognize these phosphotyrosine motifs are recruited to the receptor, and are then themselves activated by a JAK-dependent tyrosine phosphorylation event. Upon activation, STATs dissociate from the receptors, dimerize, and translocate to the nucleus to bind to specific DNA sites and alter transcription (Scott, M. J., C. J. Godshall, et al. (2002). “JAKs, STATs, Cytokines, and Sepsis.” Clin Diagn Lab Immunol 9(6): 1153-9).

The JAK family plays a role in the cytokine-dependent regulation of proliferation and function of cells involved in immune response. The JAK/STAT pathway, and in particular all four members of the JAK family, are believed to play a role in the pathogenesis of the asthmatic response, chronic obstructive pulmonary disease, bronchitis, and other related inflammatory diseases of the lower respiratory tract. Moreover, multiple cytokines that signal through JAK kinases have been linked to inflammatory diseases or conditions of the upper respiratory tract such as those affecting the nose and sinuses (e.g. rhinitis, sinusitis) whether classically allergic reactions or not. The JAK/STAT pathway has also been implicated to play a role in inflammatory diseases/conditions of the eye including, but not limited to, iritis, uveitis, scleritis, conjunctivitis, as well as chronic allergic responses. Therefore, inhibition of JAK kinases may have a beneficial role in the therapeutic treatment of these diseases.

Blocking signal transduction at the level of the JAK kinases holds promise for developing treatments for human cancers. Inhibition of the JAK kinases is also envisioned to have therapeutic benefits in patients suffering from skin immune disorders such as psoriasis, and skin sensitization.

The term “JAKs inhibitor” as used herein, refers to any naturally occurring, synthetic, or semi-synthetic compound that can inhibit the activity of one or more Janus kinases (JAKs), e.g., JAK1, JAK2, JAK3, or Tyk2. In some embodiments, the JAK inhibitor selectively inhibits the activity of only one JAK, e.g., JAK1, JAK2, JAK3, or Tyk2. In some embodiments, the JAK inhibitor can inhibit the activity of more than one JAK, e.g., JAK1 and JAK2 (e.g., ruxolitinib, baricitinib, CYT387, TG101348, AZD1480); JAK2 and JAK3; JAK1 and Tyk2; JAK2 and Tyk2; HAK3 and Tyk2. Exemplary JAK inhibitors include those described generically and specifically herein. In some embodiments, the JAK inhibitor is ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 or BMS 911543. The structures of all of these JAKs inhibitors are provided below:

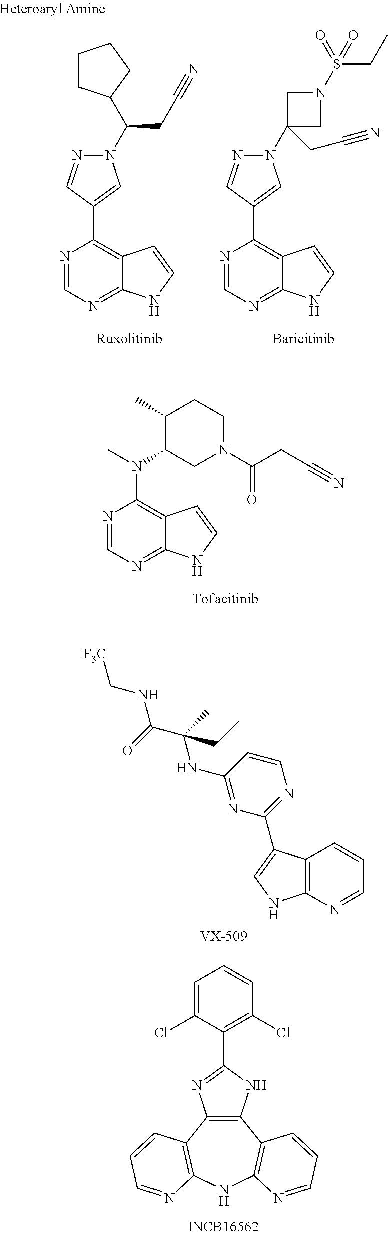

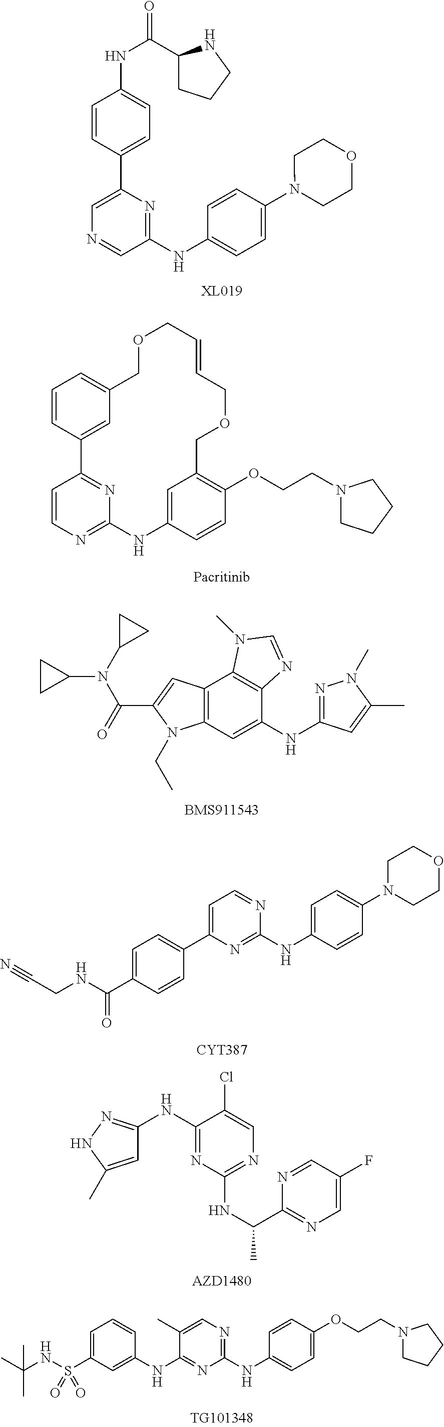

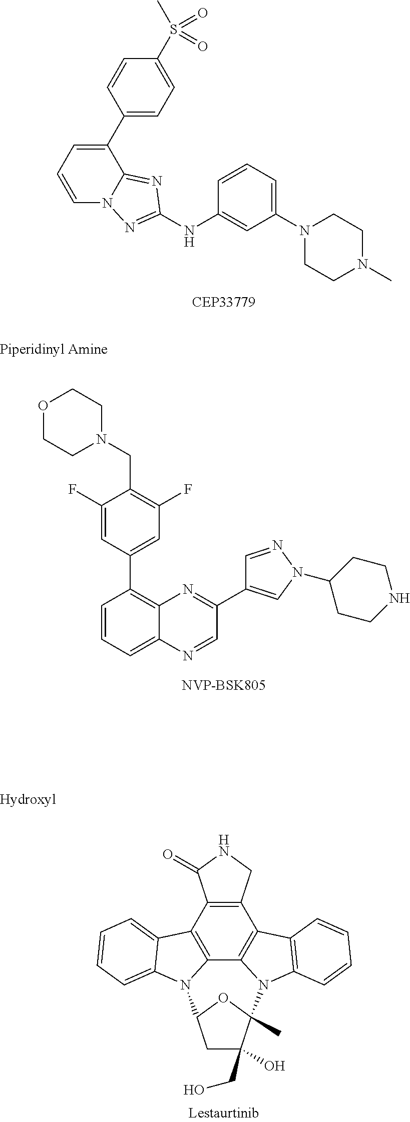

In some embodiments, the JAK inhibitor is a JAK inhibitor comprising a heteroaryl amine moiety (e.g., ruxolitinib, baricitinib, tofacitinib, VX-509, INCB16562, XL019, pacritinib, BMS911543, CYT387, ACD1480, TG101348, or CEP33779). In certain such embodiments, the JAK inhibitor is a JAK inhibitor comprising a pyrrolopyrimidine moiety (e.g., ruxolitinib, baricitinib, or tofacitinib).

In some embodiments, the JAK inhibitor is a JAK inhibitor comprising a piperidinyl amine (e.g., NVP-BSK805). In some embodiments, the JAK inhibitor is a JAK inhibitor comprising a hydroxyl moiety (e.g., lestaurtinib).

### Exemplary CDP-JAK Conjugates

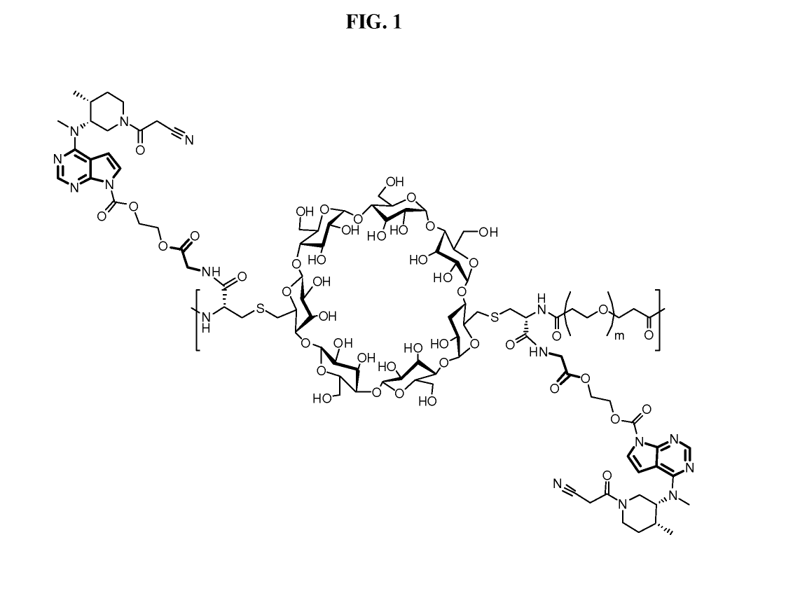

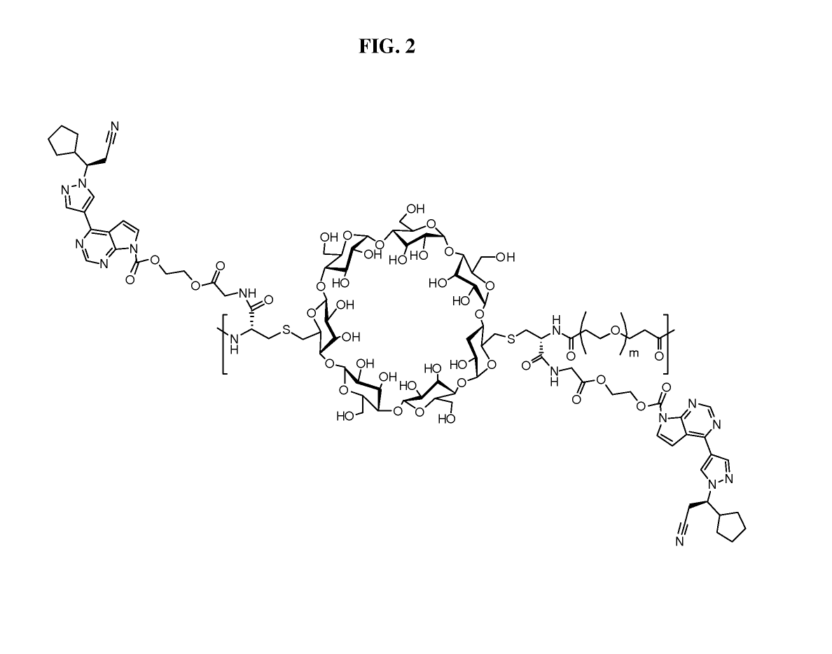

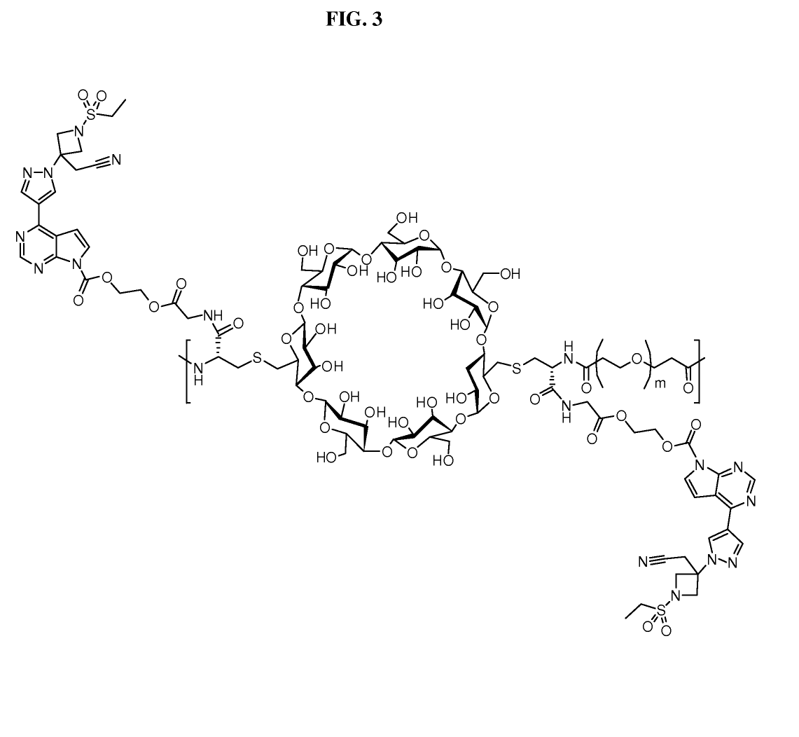

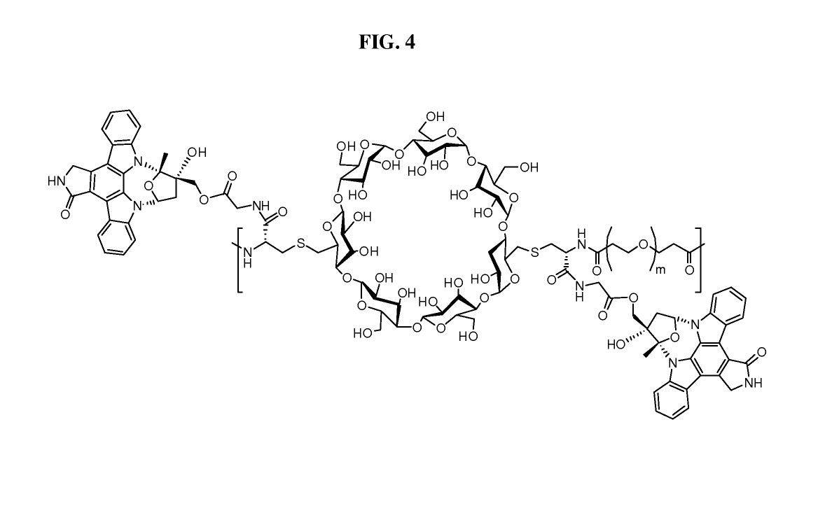

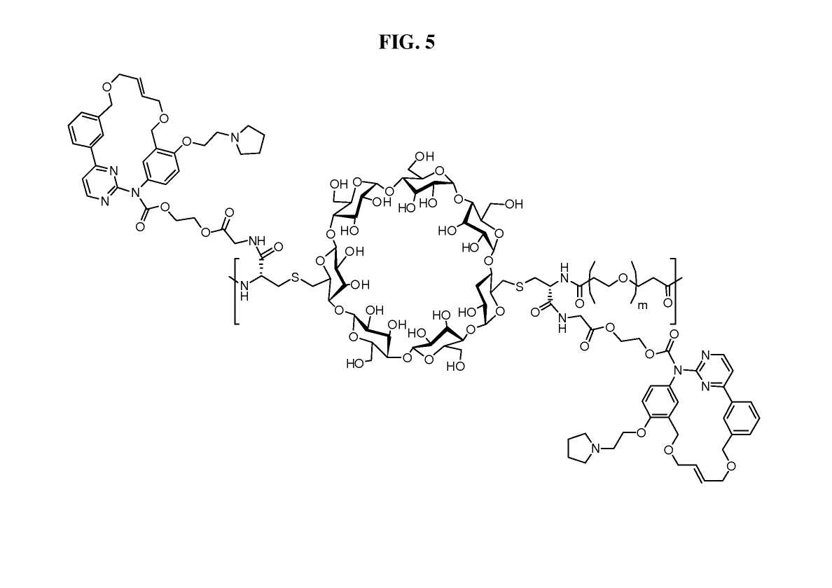

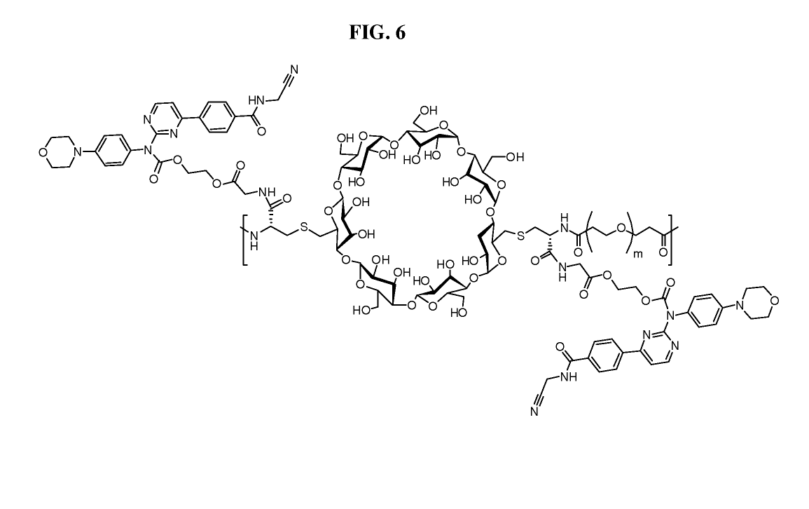

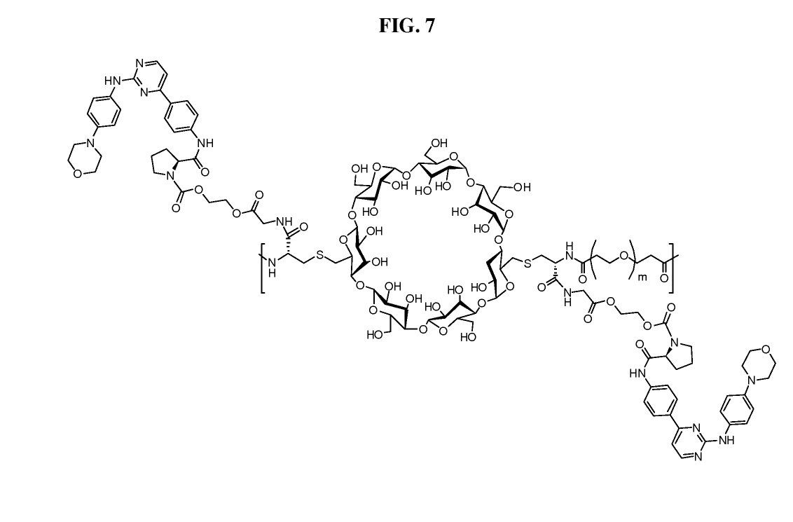

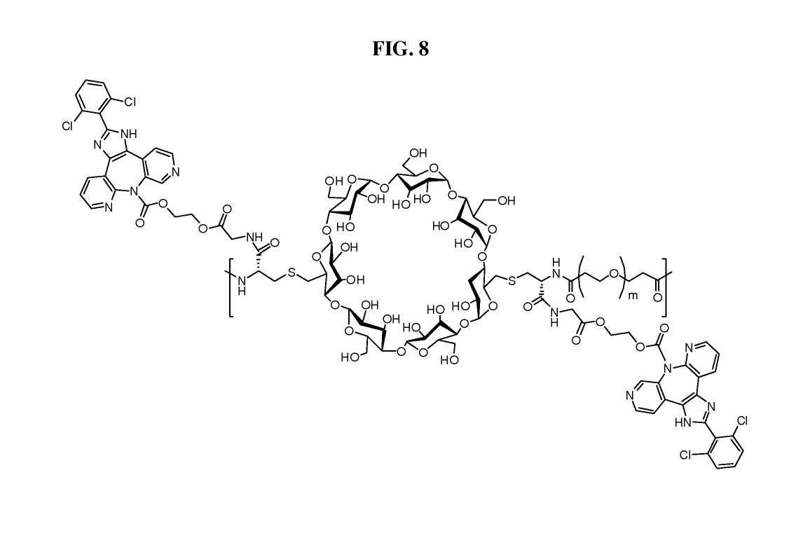

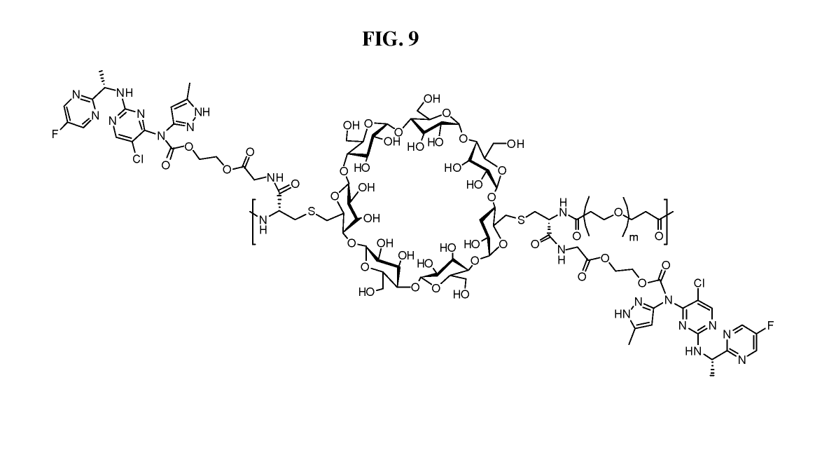

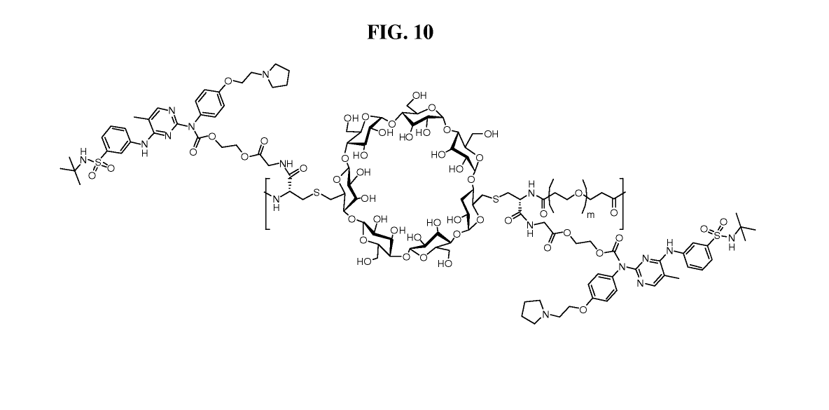

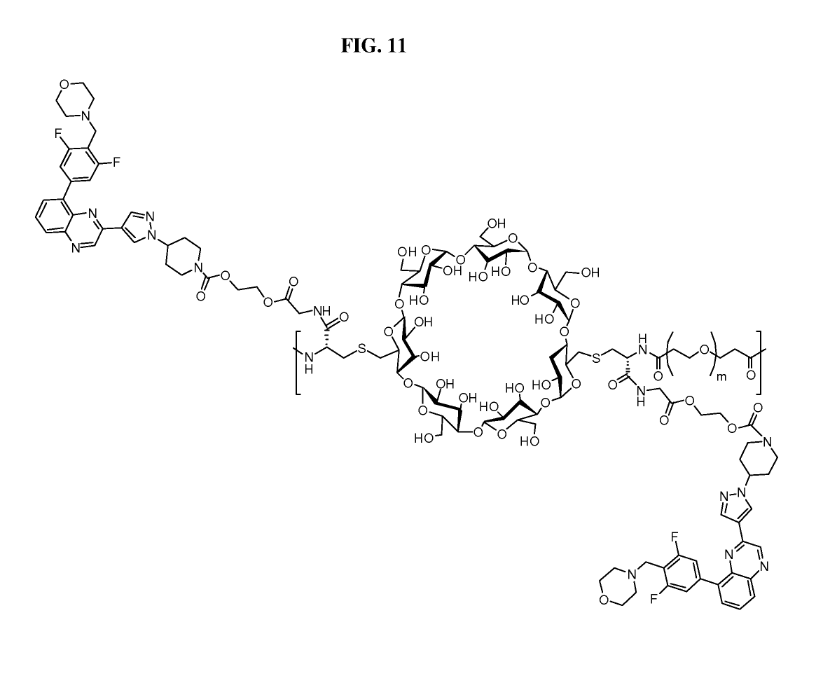

CDP-JAK inhibitor conjugates can be made using many different combinations of components described herein. For example, various combinations of cyclodextrins (e.g., beta-cyclodextrin), comonomers (e.g., PEG containing comonomers), linkers linking the cyclodextrins and comonomers, and/or linkers tethering the JAK inhibitor to the CDP are described herein. FIGS. 1-11 depict exemplary CDP-JAK inhibitor conjugates. FIG. 1 depicts a CDP-tofacitinib conjugate. FIG. 2 depicts a CDP-ruxolitinib conjugate. FIG. 3 depicts a CDP-baricitinib conjugate. FIG. 4 depicts a CDP-lestauritinib conjugate. FIG. 5 depicts a CDP-pacritinib conjugate. FIG. 6 depicts a CDP-CYT387 conjugate. FIG. 7 depicts a CDP-XL019 conjugate. FIG. 8 depicts a CDP-INCB16562 conjugate. FIG. 9 depicts a AZD1480 conjugate. FIG. 10 depicts a CDP-TG101348 conjugate. FIG. 11 depicts a CDP-NVP-BSK805 conjugate.

An exemplary cyclodextrin containing polymer (CDP) is shown below:

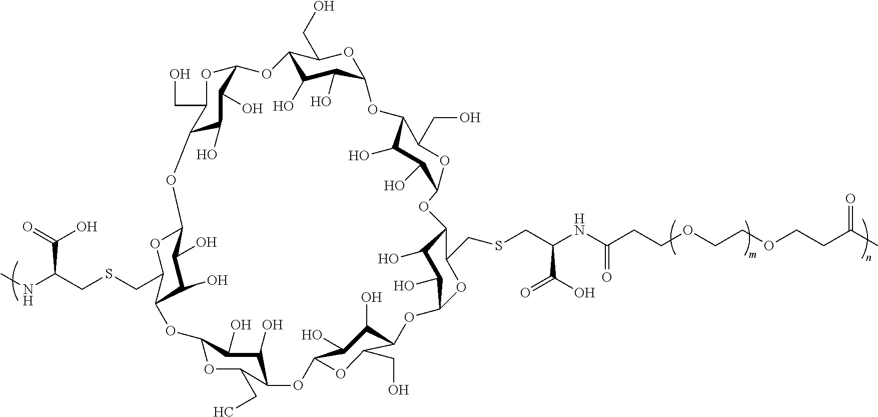

wherein the group

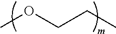

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20. Note that the JAK inhibitor is conjugated to the CDP through the carboxylic acid moieties of the polymer as provided above. Full loading of the JAK inhibitor onto the CDP is not required. In some embodiments, at least one, e.g., at least 2, 3, 4, 5, 6 or 7, of the carboxylic acid moieties remains unreacted with the JAK inhibitor after conjugation (e.g., a plurality of the carboxylic acid moieties remain unreacted).

### CDP-JAK Inhibitor Conjugate Characteristics

In some embodiments, the CDP and/or CDP-JAK inhibitor conjugates as described herein have polydispersities less than about 3, or even less than about 2.

One embodiment of the disclosure provides an improved delivery of certain JAK inhibitors by covalently conjugating them to a CDP. Such conjugation improves the aqueous solubility and hence the bioavailability of the JAK inhibitor. Accordingly, in one embodiment of the disclosure, the JAK inhibitor is a hydrophobic compound with a log P>0.4, >0.6, >0.8, >1, >2, >3, >4, or even >5. In other embodiments, a JAK inhibitor may be attached to another compound, such as an amino acid, prior to covalently attaching the conjugate onto the CDP.

The CDP-JAK inhibitor conjugates described herein preferably have molecular weights in the range of 10,000 to 500,000; 30,000 to 200,000; or even 70,000 to 150,000 amu. In certain embodiments as disclosed herein, the compound has a number average (Mₙ) molecular weight between 1,000 to 500,000 amu, or between 5,000 to 200,000 amu, or between 10,000 to 100,000 amu. One method to determine molecular weight is by gel permeation chromatography (“GPC”), e.g., mixed bed columns, CH₂Cl₂ solvent, light scattering detector, and off-line do/dc. Other methods are known in the art.

In certain embodiments as disclosed herein, the CDP-JAK inhibitor conjugate is biodegradable or bioerodable.

In certain embodiments as disclosed herein, the JAK inhibitor or prodrug thereof makes up at least 3% (e.g., at least about 5%, 10%, 15%, or 20%) by weight of the compound. In certain embodiments, the JAK inhibitor or prodrug thereof makes up at least 15% or 20% by weight of the compound (e.g., from 17-21% by weight).

In other embodiments, the CDP-JAK inhibitor conjugate may be a flexible or flowable material. When the CDP used is itself flowable, the CDP composition of the disclosure, even when viscous, need not include a biocompatible solvent to be flowable, although trace or residual amounts of biocompatible solvents may still be present.

When a solvent is used to facilitate mixing or to maintain the flowability of the CDP-JAK inhibitor conjugate, it should be non-toxic, otherwise biocompatible, and should be used in relatively small amounts. Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

In certain embodiments, the CDP-JAK inhibitor conjugates are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

In certain embodiments, the CDP-JAK inhibitor conjugates described herein, upon contact with body fluids, undergo gradual degradation. The life of a biodegradable polymer in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with a JAK inhibitoror other material, release of the JAK inhibitor or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the JAK inhibitor or any other material associated with the polymer.

A variety of factors may affect the desired rate of hydrolysis of CDP-JAK inhibitor conjugates, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the disclosure contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as a JAK inhibitor or other material loaded in the CDP-JAK inhibitor conjugates of the disclosure involves degradation of any such matrix in a 0.1 M PBS solution (pH 7.4) at 37° C., an assay known in the art. For purposes of the disclosure, the term “PBS protocol” is used herein to refer to such protocol.

In certain instances, the release rates of different CDP-JAK inhibitor conjugates of the disclosure may be compared by subjecting them to such a protocol. In certain instances, it may be necessary to process polymeric systems in the same fashion to allow direct and relatively accurate comparisons of different systems to be made. For example, the disclosure teaches several different methods of formulating the CDP-JAK inhibitor conjugates. Such comparisons may indicate that any one CDP-JAK inhibitor conjugate releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the disclosure and release rate protocols.

In certain embodiments, when formulated in a certain manner, the release rate for CDP-JAK inhibitor conjugates of the disclosure may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any incorporated material may be 10,000 ng/day/mg, or even higher. In certain instances, materials incorporated and characterized by such release rate protocols may include therapeutic agents, fillers, and other substances.

In another aspect, the rate of release of any material from any CDP-JAK inhibitor conjugate of the disclosure may be presented as the half-life of such material in the matrix.

In addition to the embodiment involving protocols for in vitro determination of release rates, in vivo protocols, whereby in certain instances release rates for polymeric systems may be determined in vivo, are also contemplated by the disclosure. Other assays useful for determining the release of any material from the polymers of the present system are known in the art.

### Physical Structures of the CDP-JAK Inhibitor Conjugates

The CDP-JAK inhibitor conjugates may be formed in a variety of shapes. For example, in certain embodiments, the CDP-JAK inhibitor conjugates may be presented in the form of a nanoparticle. In one embodiment, the CDP-JAK inhibitor conjugate self assembles into a nanoparticle. In one embodiment, the CDP-JAK inhibitor conjugate self assembles into a nanoparticle in an aqueous solution, e.g., water.

In addition to intracellular delivery of a JAK inhibitor, it also possible that nanoparticles of the CDP-JAK inhibitor conjugates may undergo endocytosis, thereby obtaining access to the cell. The frequency of such an endocytosis process will likely depend on the size of any nanoparticle.

In one embodiment, the surface charge of the molecule is neutral, or slightly negative. In some embodiments, the zeta potential of the particle surface is from about −80 mV to about 50 mV.

### Particles: Conjugate Number

Conjugate number, as used herein, is the number of cyclodextrin containing polymer (“CDP”) therapeutic agent conjugate molecules, present in a particle or nanoparticle. For purposes of determining conjugate number, a particle or nanoparticle is an entity having one, or typically, more than one CDP therapeutic agent conjugate molecules, which, at the concentration suitable for administration to humans, behaves as a single unit in any of water, e.g., water at neutral pH, PBS, e.g., PBS at pH 7.4, or in a formulation in which it will be administered to patients. For purposes of calculating conjugate number, a CDP therapeutic agent (e.g., JAK inhibitor) conjugate molecule is a single CDP polymer with its covalently linked therapeutic agent (e.g., JAK inhibitor).

Methods disclosed herein provide for evaluating a particle, e.g., a nanoparticle, or preparation of particles, e.g., nanoparticles, wherein said particles, e.g., nanoparticles, comprise a CDP therapeutic agent (e.g., JAK inhibitor) conjugate. Generally, the method comprises providing a sample comprising a plurality of said particles, e.g., nanoparticles, determining a value for the number of CDP therapeutic agent (e.g., JAK inhibitor) conjugates in a particle, e.g., nanoparticle, in the sample, to thereby evaluate a preparation of particles, e.g., nanoparticles.

Typically the value for a particle will be a function of the values obtained for a plurality of particles, e.g., the value will be the average of values determined for a plurality of particles.

In embodiments the method further comprises comparing the determined value with a reference value. The comparison can be used in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. E.g., based on the result of the determination, or upon comparison to a reference standard, the batch from which the sample is taken can be processed, e.g., as just described.

As discussed above, conjugate number is defined as the number of CDP-therapeutic agent (e.g., JAK inhibitor) conjugate molecules that self-assemble into a particle or nanoparticle, thus

C_{J}=[CDP-therapeutic agent (e.g., JAK inhibitor) conjugate]/P (or NP)

where Cj is conjugate number, [CDP-therapeutic agent (e.g., JAK inhibitor) conjugate]/is the number of CDP-therapeutic agent (e.g., JAK inhibitor) conjugate molecules, and P (or NP) is a single particle (or nanoparticle).

In order to arrive and conjugate number one determines the size of a particle, e.g., by dynamic light scattering. The size should be viscosity-adjusted size. The hydrodynamic volume of a CDP-therapeutic agent (e.g., JAK inhibitor) conjugate, or a molecule of similar molecular weight, is determined, to provide an expected hydrodynamic volume. Comparison of the expected hydrodynamic volume for the CDP-therapeutic agent conjugate with the volume for a particle of determined size provides conjugate number.

The determination of conjugate number is demonstrated with CRLX101, in which camptothecin is coupled to the CDP backbone. In the case of CRLX101, a number of fundamental assumptions are made in postulating nanoparticle characteristics. First, macromolecular volume estimates are based on work done with bovine serum albumin (BSA), a biological macromolecule of similar size to CRLX101 (BSA MS=67 kDa, 101 MW=66.5 kDa). It has been demonstrated that a single strand of BSA has a hydrodynamic diameter of 9.5 nm. Simple volume calculations yield a volume of 3589 nm³. Extending this to CRLX 101 with an average 30 nm particle, gives a volume of 33,485 nm³. With a particle size of 5-40 nm the conjugate number is 1-30. FIG. 1 shows a calculated strand dependence on particle size.

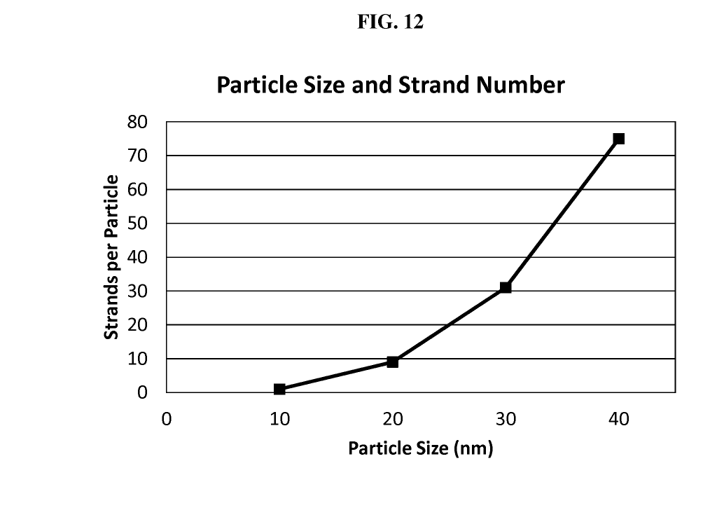

Given the particle size distribution of CRLX101, the conjugate number can range from 30-75, as shown in FIG. 12.

Polymer Polydispersity. CRLX101 molecules fall within a range of molecular weights, with molecules of varying weight providing varying contributions to the particle diameter and conjugate number. Particles could form which are made up of strands which are larger and smaller than the average. Strands may also associate to a maximum size which could be shear-limited.

Particle Shape. Particle shape is assumed to be roughly spherical, and driven by either (or both) the hydrophobic region created by the CDP-therapeutic agent (e.g., JAK inhibitor) conjugate, or by guest-host complexation with pendant therapeutic agent molecules making inclusion complexes with CDs from adjacent strands. One critical point of note is that as a drug product, the NPs are in a somewhat controlled environment as they are characterized. Upon administration, myriad possibilities exist for interaction with endogenous substances: inclusion complexes of circulating small molecules, metal ion complexation with the PEG subunits, etc. Any one of these are all of them in concert could dramatically alter the NP structure and function.

### CDPs, Methods of Making Same, and Methods of Conjugating CDPs to JAK Inhibitors

The CDP-JAK Inhibitor conjugates described herein can be prepared by covalently attaching one or more JAK inhibitors to a CDP.

Another aspect of the disclosure is a method for manufacturing the linear CDPs and CDP-JAK inhibitor conjugates as described herein.

Accordingly, one embodiment of the disclosure is a method of preparing a linear CDP. A linear CDP may be prepared by copolymerizing a cyclodextrin monomer precursor disubstituted with one or more appropriate leaving groups with a comonomer precursor capable of displacing the leaving groups. The leaving group, which may be the same or different, may be any leaving group known in the art which may be displaced upon copolymerization with a comonomer precursor. In a preferred embodiment, a linear CDP may be prepared by iodinating a cyclodextrin monomer precursor to form a diiodinated cyclodextrin monomer precursor and copolymerizing the diiodinated cyclodextrin monomer precursor with a comonomer precursor to form a linear CDP having a repeating unit of formula I or II, provided in the section entitles “CDP-JAK inhibitor conjugates” or a combination thereof, each as described above. In some embodiments, the cyclodextrin moiety precursors are in a composition, the composition being substantially free of cyclodextrin moieties having other than two positions modified to bear a reactive site (e.g., 1, 3, 4, 5, 6, or 7). While examples presented below discuss iodinated cyclodextrin moieties, one skilled in the art would readily recognize that the disclosure contemplates and encompasses cyclodextrin moieties wherein other leaving groups such as alkyl and aryl sulfonate may be present instead of iodo groups. In a preferred embodiment, a method of preparing a linear cyclodextrin copolymer of the disclosure by iodinating a cyclodextrin monomer precursor as described above to form a diiodinated cyclodextrin monomer precursor of formula IVa, IVb, IVc or a mixture thereof:

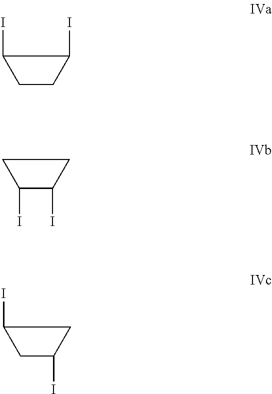 [SMILES: IC1CCC1I ; IC1CCC1I ; IC1CC(I)C1]

In some embodiments, the iodine moieties as shown on the cyclodextrin moieties are positioned such that the derivatization on the cyclodextrin is on the A and D glucopyranose moieties. In some embodiments, the iodine moieties as shown on the cyclodextrin moieties are positioned in such that the derivatization on the cyclodextrin is on the A and C glucopyranose moieties. In some embodiments, the iodine moieties as shown on the cyclodextrin moieties are positioned in such that the derivatization on the cyclodextrin is on the A and F glucopyranose moieties. In some embodiments, the iodine moieties as shown on the cyclodextrin moieties are positioned in such that the derivatization on the cyclodextrin is on the A and E glucopyranose moieties.

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also according to the disclosure, the iodo groups or other appropriate leaving group may be displaced with a group that permits reaction with a comonomer precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

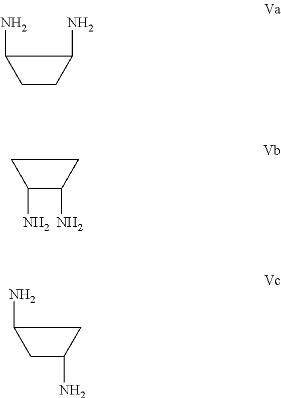 [SMILES: NC1CCC1N ; NC1CCC1N ; NC1CC(N)C1]

In some embodiments, the amino moieties as shown on the cyclodextrin moieties are positioned such that the derivatization on the cyclodextrin is on the A and D glucopyranose moieties. In some embodiments, the amino moieties as shown on the cyclodextrin moieties are positioned in such that the derivatization on the cyclodextrin is on the A and C glucopyranose moieties. In some embodiments, the amino moieties as shown on the cyclodextrin moieties are positioned in such that the derivatization on the cyclodextrin is on the A and F glucopyranose moieties. In some embodiments, the amino moieties as shown on the cyclodextrin moieties are positioned in such that the derivatization on the cyclodextrin is on the A and E glucopyranose moieties.

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-β-cyclodextrin). The cyclodextrin monomer precursor is aminated at only two positions. The diaminated cyclodextrin monomer precursor may then be copolymerized with a comonomer precursor, as described above, to produce a linear cyclodextrin copolymer having a repeating unit of formula I-II provided in the section entitles “CDP-JAK inhibitor conjugates” or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

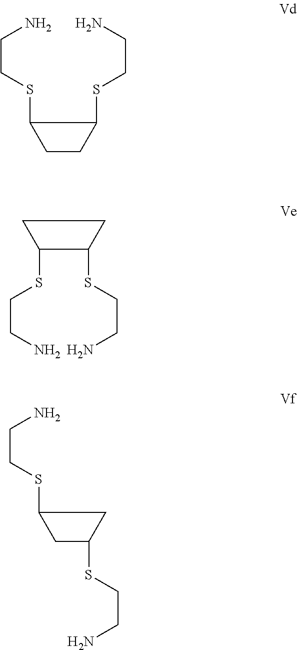 [SMILES: NCCSC1CCC1SCCN ; NCCSC1CCC1SCCN ; NCCSC1CC(SCCN)C1]

In some embodiments, the —SCH₂CH₂NH₂ moieties as shown on the cyclodextrin moieties are positioned such that the derivatization on the cyclodextrin is on the A and D glucopyranose moieties. In some embodiments, the —SCH₂CH₂NH₂ moieties as shown on the cyclodextrin moieties are positioned in such that the derivatization on the cyclodextrin is on the A and C glucopyranose moieties. In some embodiments, the —SCH₂CH₂NH₂ moieties as shown on the cyclodextrin moieties are positioned in such that the derivatization on the cyclodextrin is on the A and F glucopyranose moieties. In some embodiments, the —SCH₂CH₂NH₂ moieties as shown on the cyclodextrin moieties are positioned in such that the derivatization on the cyclodextrin is on the A and E glucopyranose moieties.

In some embodiments, a CDP comprises: cyclodextrin moieties, and comonomers which do not contain cyclodextrin moieties (comonomers), and wherein the CDP comprises at least four, five six, seven, eight, nine, ten, eleven, twelve, thirteen, fourteen, fifteen, sixteen, seventeen, eighteen, nineteen or twenty cyclodextrin moieties and at least four, five six, seven, eight, nine, ten, eleven, twelve, thirteen, fourteen, fifteen, sixteen, seventeen, eighteen, nineteen or twenty comonomers.

In some embodiments, the at least four, five six, seven, eight, etc., cyclodextrin moieties and at least four, five six, seven, eight, nine, ten, eleven, twelve, thirteen, fourteen, fifteen, sixteen, seventeen, eighteen, nineteen or twenty comonomers alternate in the water soluble linear polymer.

In some embodiments, the cyclodextrin moieties comprise linkers to which therapeutic agents may be further linked.

In some embodiments, the CDP has no JAK inhibitors attached. In some embodiments, the CDP has a plurality (i.e., more than one) of JAK inhibitors attached (e.g., through a linker). In some embodiments, the JAK inhibitors are attached via a second linker.

In some embodiments, the comonomer is a compound containing residues of least two functional groups through which reaction and thus linkage of the cyclodextrin monomers is achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the residues of the two functional groups are the same and are located at termini of the comonomer. In some embodiments, a comonomer contains one or more pendant groups with at least one functional group through which reaction and thus linkage of a JAK inhibitor can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient JAK inhibitor such that up to at least 5%, 10%, 15%, 20%, 25%, 30%, or even 35% by weight of the water soluble linear polymer, when conjugated, is JAK inhibitor.

In some embodiments, the molecular weight of the CDP is 10,000-500,000 Da, e.g., about 30,000 to about 100,000 Da.

In some embodiments, the cyclodextrin moieties make up at least about 2%, 5%, 10%, 11%, 12%, 13%, 14%, 15%, 16%, 17%, 18%, 19%, 20%, 30%, 50% or 80% of the polymer by weight.

In some embodiments, the CDP is made by a method comprising providing cyclodextrin moiety precursors modified to bear one reactive site at each of exactly two positions, and reacting the cyclodextrin moiety with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and comonomer is produced.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain.

In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP is a polymer of the following formula:

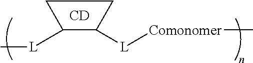

wherein each L is independently a linker, each comonomer is independently a comonomer described herein, and n is at least 4, 5, 6, 7, 8, 9, 10, 11, 12, 13, 14, 15, 16, 17, 18, 19 or 20. In some embodiments, the molecular weight of the comonomer is from about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)).

CD can, in some embodiments, be replaced by a polyols. Exemplary polyols include, for example, mucic acid and trehalose.

In some embodiments, the CDP is a polymer of the following formula:

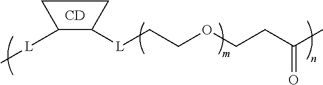

wherein each L is independently a linker,

wherein the group

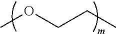

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments,

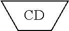

is alpha, beta or gamma cyclodextrin, e.g., beta cyclodextrin.

In some embodiments, each L independently comprises an amino acid or a derivative thereof. In some embodiments, at least one L comprises cysteine or a derivative thereof. In some embodiments, each L comprises cysteine. In some embodiments, each L is cysteine and the cysteine is connected to the CD by way of a thiol linkage.

In some embodiments, the CDP is a polymer of the following formula:

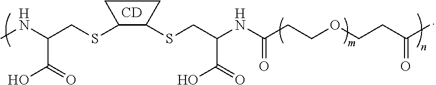

wherein the group

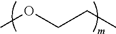

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments,

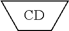

is alpha, beta or gamma cyclodextrin, e.g., beta cyclodextrin.

In some embodiments, the CDP is a polymer of the following formula:

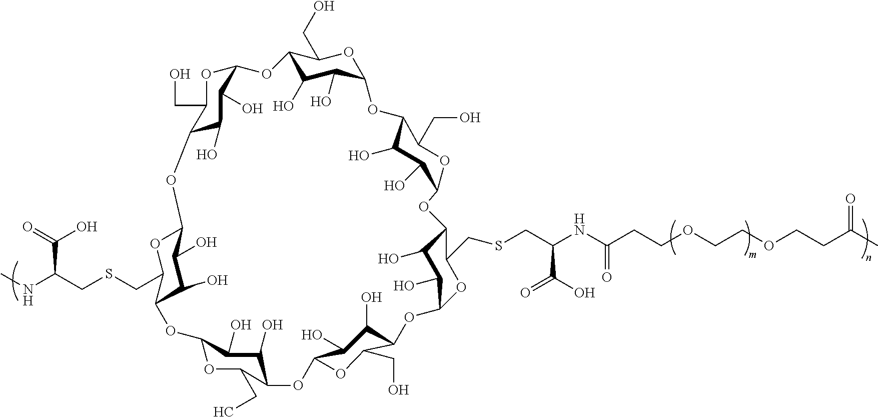

wherein the group

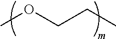

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, the group

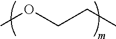

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and the Mw of the compound as a whole is from 27 kDa to 99.6 kDa.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four, five six, seven, eight, or more, cyclodextrin moieties and at least four, five six, seven, eight, or more, comonomers.

In some embodiments, the at least four, five, six, seven, eight, or more cyclodextrin moieties and at least four, five, six, seven, eight, or more comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin comonomers comprise linkers to which JAK inhibitors may be further linked. In some embodiments, the JAK inhibitors are linked via second linkers.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient JAK inhibitor such that up to at least 3%, 5%, 10%, 15%, 20%, 25%, 30%, or even 35% by weight of the CDP, when conjugated, is JAK inhibitor.

In some embodiments, the CDP has a molecular weight of 10,000-500,000. In some embodiments, the cyclodextrin moieties make up at least about 2%, 5%, 10%, 20%, 30%, 50% or 80% of the CDP by weight.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain. the CDP comprises a comonomer selected from the group consisting of a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP of the following formula can be made by the scheme below:

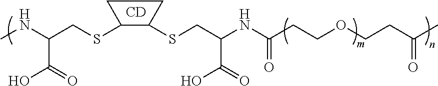

providing a compound of formula A and formula B:

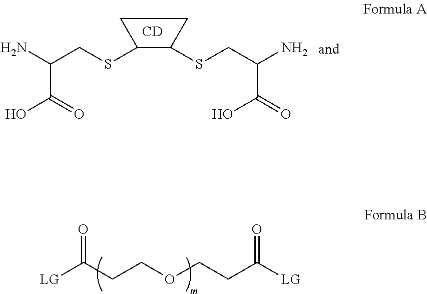

wherein LG is a leaving group;
and contacting the compounds under conditions that allow for the formation of a covalent bond between the compounds of formula A and B, to form a polymer of the following formula:

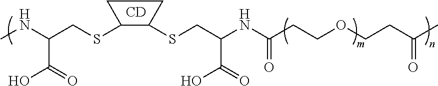

wherein the group

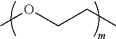

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least four.

In some embodiments, Formula B is

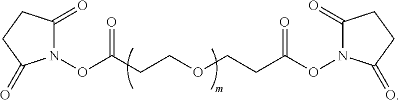

In some embodiments, the group

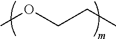

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and the Mw of the compound is from 27 kDa to 99.6 kDa.

In some embodiments, the compounds of formula A and formula B are contacted in the presence of a base. In some embodiments, the base is an amine containing base. In some embodiments, the base is DEA.

In some embodiments, a CDP of the following formula can be made by the scheme below:

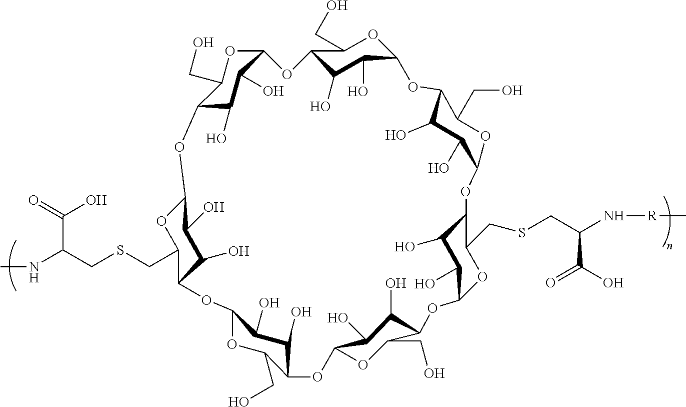

wherein R is of the form:

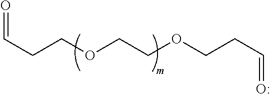

comprising the steps of:

reacting a compound of the formula below:

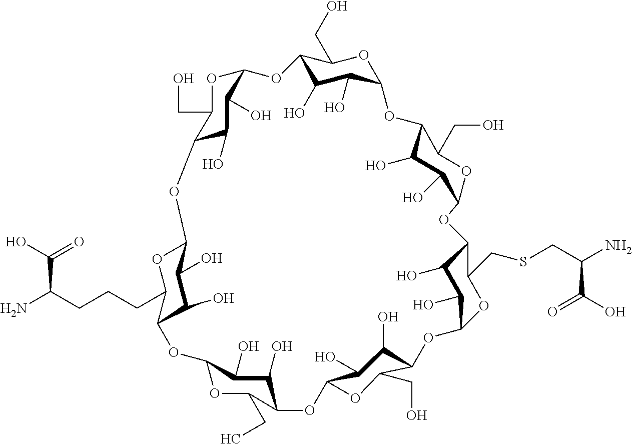 [SMILES: CCC1O[C@H]2O[C@@H]3C(CCC[C@@H](N)C(=O)O)O[C@@H](O[C@@H]4C(CO)O[C@H](O[C@@H]5C(CO)O[C@H](O[C@@H]6C(CO)O[C@H](O[C@@H]7C(CSC[C@@H](N)C(=O)O)O[C@H](O[C@@H]8C(CO)O[C@@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O]

with a compound of the formula below:

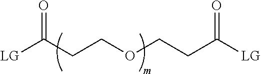

wherein the group

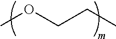

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least four,

in the presence of a non-nucleophilic organic base in a solvent.

In some embodiments, is

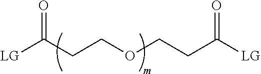

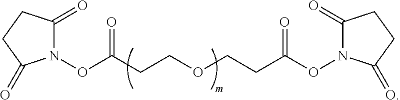

In some embodiments, the solvent is a polar aprotic solvent. In some embodiments, the solvent is DMSO.

In some embodiments, the method also includes the steps of dialysis; and lyophylization.

In some embodiments, a CDP provided below can be made by the following scheme:

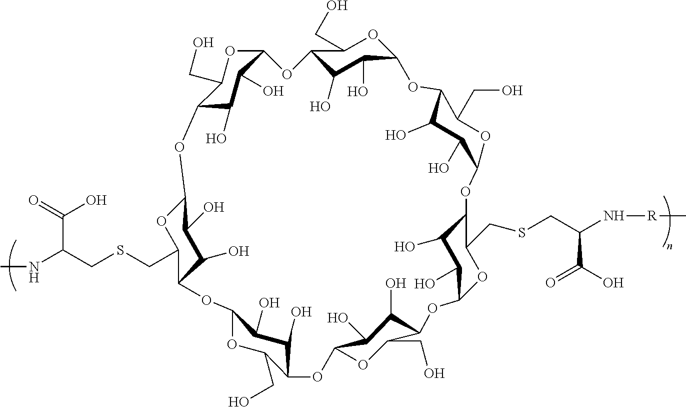

wherein R is of the form:

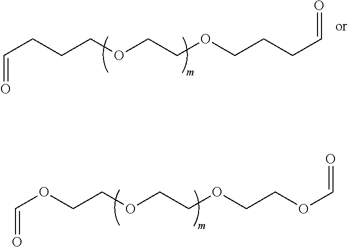

comprising the steps of:

reacting a compound of the formula below:

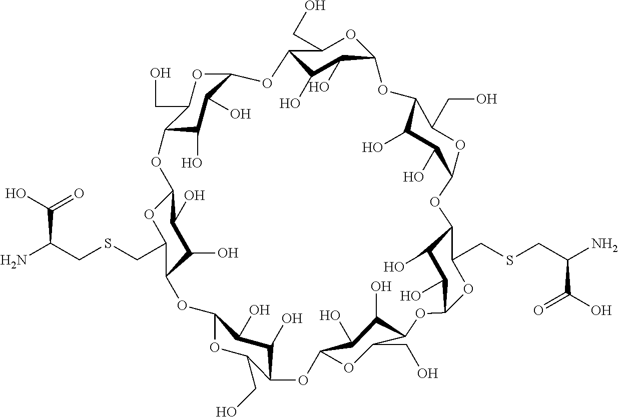 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

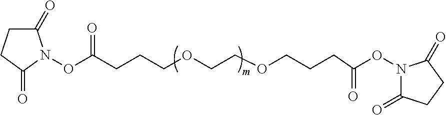

wherein the group

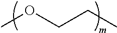

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least four,
or with a compound provided below:

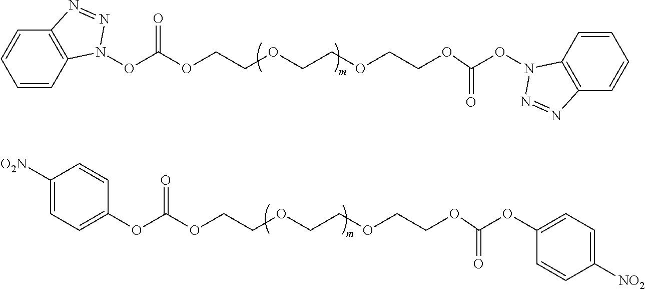

wherein the group

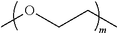

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da));
in the presence of a non-nucleophilic organic base in DMSO;

and dialyzing and lyophilizing the following polymer

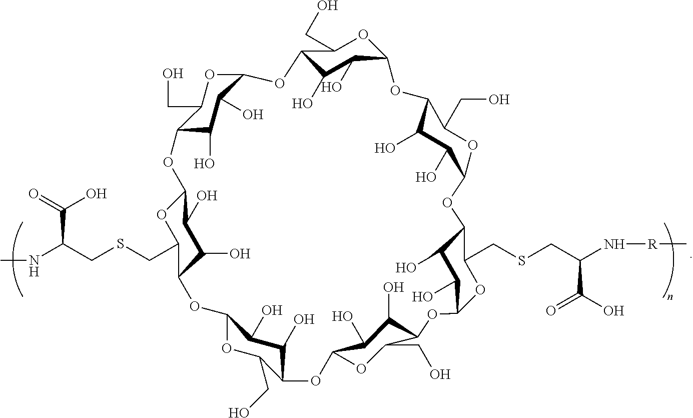

A linear CDP may be characterized by any means known in the art. Such characterization methods or techniques include, but are not limited to, gel permeation chromatography (GPC), matrix assisted laser desorption ionization-time of flight mass spectrometry (MALDI-TOF Mass spec), ¹H and ¹³C NMR, light scattering and titration.

One aspect of the disclosure contemplates attaching a JAK inhibitor to a CDP for delivery of a JAK inhibitor. In certain embodiments, the JAK inhibitor is covalently linked via a biohydrolyzable bond, for example, an ester, amide, carbamates, or carbonate.

Scheme IIb: General Scheme of Preparing Linear CDPs.

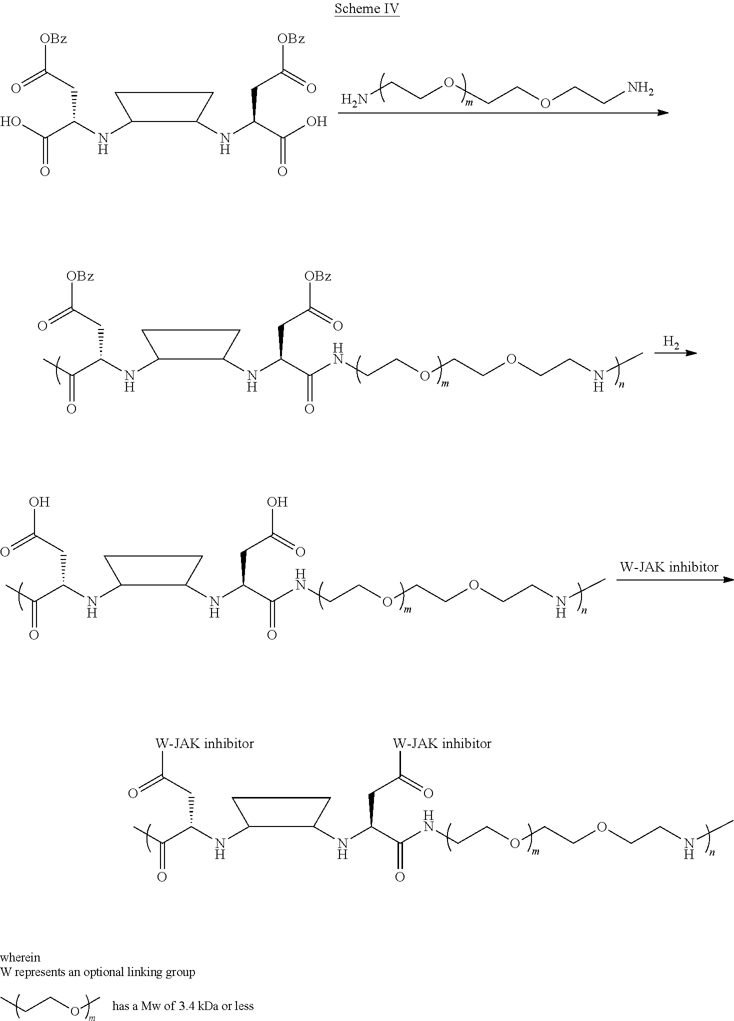

Scheme IV, as provided above, includes embodiments where W-JAK inhibitor is absent in one or more positions as provided above. This can be achieved, for example, when less than 100% yield is achieved when coupling the JAK inhibitor to the polymer and/or when less than an equivalent amount of JAK inhibitor is used in the reaction. Accordingly, the loading of the JAK inhibitor, by weight of the polymer, can vary.

The disclosure further contemplates CDPs and CDP-conjugates synthesized using CD-biscysteine monomer and a di-NHS ester such as PEG-DiSPA or PEG-BTC as shown in Scheme XIII below.

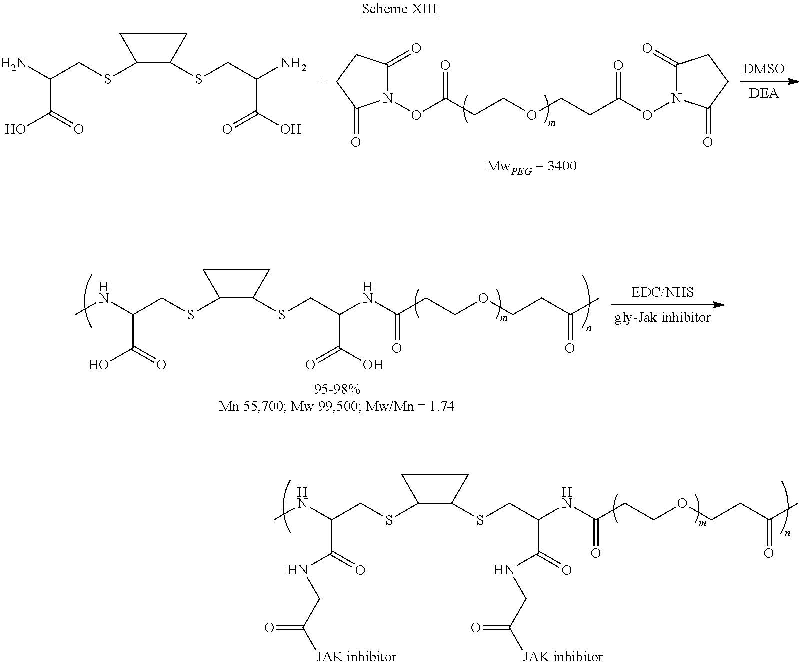

Scheme XIII, as provided above, includes embodiments where gly-JAK inhibitor is absent in one or more positions as provided above. This can be achieved, for example, when less than 100% yield is achieved when coupling the JAK inhibitor to the polymer and/or when less than an equivalent amount of JAK inhibitor is used in the reaction. Accordingly, the loading of the JAK inhibitor, by weight of the polymer, can vary.

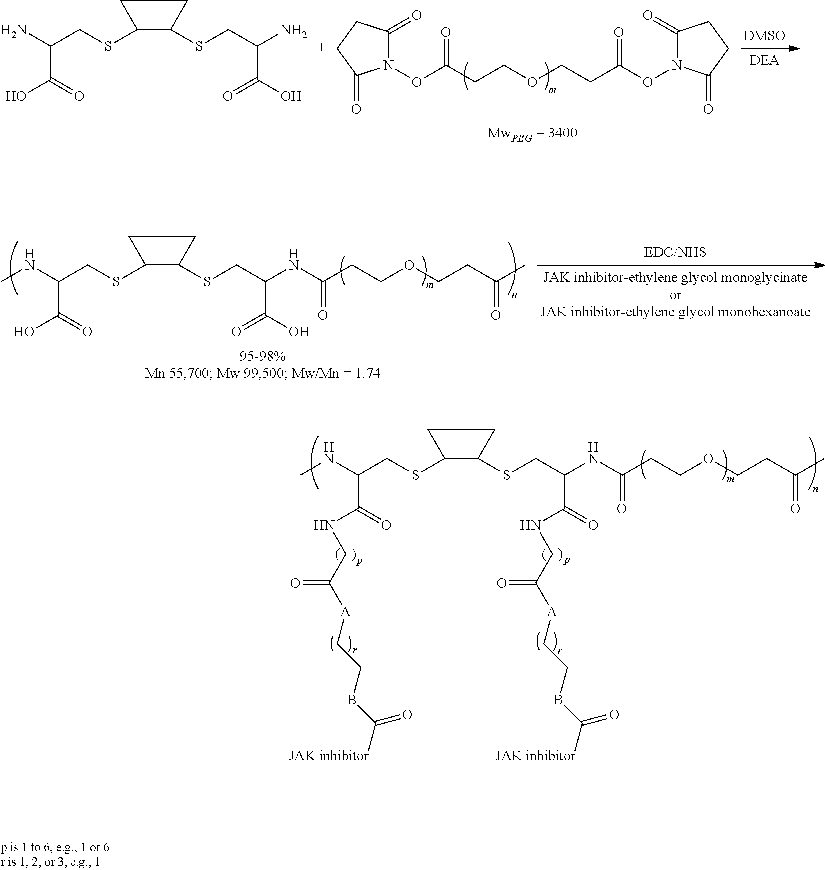

Scheme XIIIa, as provided above, includes embodiments where a JAK inhibitor is attached to the polymer through a linker that comprises a self-cyclizing moiety and a selectivity-determining moiety.

In some embodiments, the JAK inhibitor is attached via a linker. In some embodiments, the JAK inhibitor is attached to the water soluble linear polymer through an attachment that is cleaved under biological conditions to release the JAK inhibitor. In some embodiments, the JAK inhibitor is attached to the water soluble linear polymer at a cyclodextrin moiety or a comonomer. In some embodiments, the JAK inhibitor is attached to the water soluble linear polymer via an optional linker to a cyclodextrin moiety or a comonomer.

In some embodiments, the cyclodextrin moieties comprise linkers to which therapeutic agents are linked. In some embodiments, the cyclodextrin moieties comprise linkers to which therapeutic agents are linked via a second linker.

In some embodiments, the CDP is made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. In some embodiments, the CDP is conjugated with a JAK inhibitor to provide a CDP-JAK inhibitor conjugate.

In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the JAK inhibitor is attached to the CDP via a linker. In some embodiments, the linker is cleaved under biological conditions.

In some embodiments, the JAK inhibitor makes up at least 5%, 10%, 15%, 20%, 25%, 30%, or even 35% by weight of the CDP-JAK inhibitor conjugate. In some embodiments, at least about 50% of available positions on the CDP are reacted with a JAK inhibitor and/or a linker JAK inhibitor (e.g., at least about 55%, 60%, 65%, 70%, 75%, 80%, 85%, 90%, or 95%).

In some embodiments, the comonomer comprises polyethylene glycol of molecular weight 3,400 Da, the cyclodextrin moiety comprises beta-cyclodextrin, the theoretical maximum loading of JAK inhibitor on the CDP-JAK inhibitor conjugate is 19%, and JAK inhibitor is 17-21% by weight of the CDP-JAK inhibitor conjugate. In some embodiments, about 80-90% of available positions on the CDP are reacted with a JAK inhibitor and/or a linker JAK inhibitor.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent is achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring.

In some embodiments, the cyclodextrin moiety comprises an alpha, beta, or gamma cyclodextrin moiety.

In some embodiments, the JAK inhibitor is poorly soluble in water.

In some embodiments, the solubility of the JAK inhibitor is <5 mg/ml at physiological pH.

In some embodiments, the JAK inhibitor is a hydrophobic compound with a log P>0.4, >0.6, >0.8, >1, >2, >3, >4, or >5. In some embodiments, the JAK inhibitor is hydrophobic and is attached via a second compound.

In some embodiments, administration of the CDP-JAK inhibitor conjugate to a subject results in release of the JAK inhibitor over a period of at least 6 hours. In some embodiments, administration of the CDP-JAK inhibitor conjugate to a subject results in release of the JAK inhibitor over a period of 6 hours to a month. In some embodiments, upon administration of the CDP-JAK inhibitor conjugate to a subject the rate of JAK inhibitor release is dependent primarily upon the rate of hydrolysis as opposed to enzymatic cleavage.

In some embodiments, the CDP-JAK inhibitor conjugate has a molecular weight of 10,000-500,000.

In some embodiments, the cyclodextrin moieties make up at least about 2%, 5%, 10%, 20%, 30%, 50% or 80% of the polymer by weight.

In some embodiments, a the CDP includes a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, a comonomer comprises a polyglycolic acid or polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP-polymer conjugate of the following formula can be made as follows:

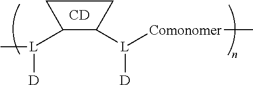

providing a polymer of the formula below:

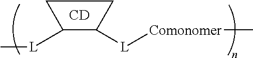

and coupling the polymer with a plurality of D moieties, wherein each D is independently absent or independently a JAK inhibitor, to provide:

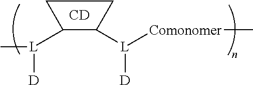

wherein the comonomer has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, one or more of the JAK inhibitor moieties in the CDP-JAK inhibitor conjugate can be replaced with another therapeutic agent, e.g., another anticancer agent or anti-inflammatory agent.

In some embodiments, a CDP-polymer conjugate of the following formula can be made as follows:

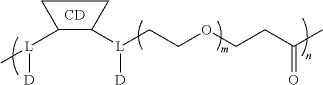

providing a polymer of the formula below:

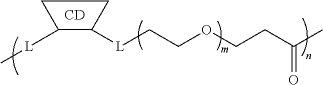

and coupling the polymer with a plurality of D moieties, wherein each D is independently absent or a JAK inhibitor, to provide:

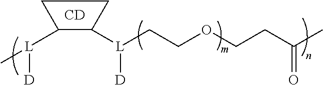

wherein the group

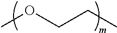

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, one or more of the JAK inhibitor moieties in the CDP-JAK inhibitor conjugate can be replaced with another therapeutic agent, e.g., another anticancer agent or anti-inflammatory agent.

The reaction scheme as provided above includes embodiments where D is absent in one or more positions as provided above. This can be achieved, for example, when less than 100% yield is achieved when coupling the JAK inhibitor to the polymer (e.g., 80-90%) and/or when less than an equivalent amount of JAK inhibitor is used in the reaction. Accordingly, the loading of the JAK inhibitor, by weight of the polymer, can vary, for example, the loading of the JAK inhibitor can be at least about 3% by weight, e.g., at least about 5%, at least about 8%, at least about 10%, at least about 13%, at least about 15%, or at least about 20%.

In some embodiments, a CDP-polymer conjugate of the following formula can be made as follows:

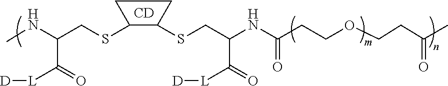

providing a polymer below:

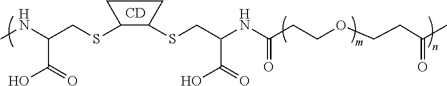

and coupling the polymer with a plurality of L-D moieties, wherein L is a linker or absent and D is a JAK inhibitor, to provide:

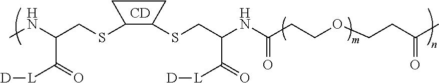

wherein the group

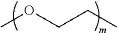

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

In some embodiments, one or more of the JAK inhibitor moieties in the CDP-JAK inhibitor conjugate can be replaced with another therapeutic agent, e.g., another anticancer agent or anti-inflammatory agent.

The reaction scheme as provided above includes embodiments where L-D is absent in one or more positions as provided above. This can be achieved, for example, when less than 100% yield is achieved when coupling the JAK inhibitor-linker to the polymer (e.g., 80-90%) and/or when less than an equivalent amount of JAK inhibitor-linker is used in the reaction. Accordingly, the loading of the JAK inhibitor, by weight of the polymer, can vary, for example, the loading of the JAK inhibitor can be at least about 3% by weight, e.g., at least about 5%, at least about 8%, at least about 10%, at least about 13%, at least about 15%, or at least about 20%.

In some embodiments, at least a portion of the L moieties of L-D is absent. In some embodiments, each L is independently an amino acid or derivative thereof (e.g., glycine).

In some embodiments, the coupling of the polymer with the plurality of L-D moieties results in the formation of a plurality of amide bonds.

In certain instances, the CDPs are random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

### Pharmaceutical Compositions

In another aspect, the disclosure provides a composition, e.g., a pharmaceutical composition, comprising a CDP-JAK inhibitor conjugate and a pharmaceutically acceptable carrier or adjuvant.

In some embodiments, a pharmaceutical composition may include a pharmaceutically acceptable salt of a CDP-JAK inhibitor conjugate, e.g., a CDP-JAK inhibitor conjugate described herein. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This disclosure also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a CDP-JAK inhibitor conjugate, which may be any liquid solution compatible with the CDP-JAK inhibitor conjugate, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, and mixtures thereof.

A composition described herein may also include another component, such as an antioxidant, antibacterial, buffer, bulking agent, chelating agent, an inert gas, a tonicity agent and/or a viscosity agent.

In one embodiment, the CDP-JAK inhibitor conjugate is provided in lyophilized form and is reconstituted prior to administration to a subject. The lyophilized CDP-JAK inhibitor conjugate can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the CDP-JAK inhibitor conjugate from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxomers, Tweens, lecithins, saccharides, oligosaccharides, polysaccharides and polyols (e.g., trehalose, mannitol, sorbitol, lactose, sucrose, glucose and dextran), salts and crown ethers.

In some embodiments, the lyophilized CDP-JAK inhibitor conjugate is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution having a concentration of about 2 mg/mL to about 4 mg/mL of the CDP-JAK inhibitor conjugate. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the disclosure to package the subject CDP-JAK inhibitor conjugate with a third vial containing sufficient parenteral diluent to prepare the final concentration for administration. A typical diluent is Lactated Ringer's Injection.

The final dilution of the reconstituted CDP-JAK inhibitor conjugate may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulzation, propellant or a dry powder device) or via an implanted reservoir.

Pharmaceutical compositions suitable for parenteral administration comprise one or more CDP-JAK inhibitor conjugate(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the CDP-JAK inhibitor conjugate then depends upon its rate of dissolution which, in turn, may depend upon crystal size and crystalline form. Alternatively, delayed absorption of a parenterally administered drug form is accomplished by dissolving or suspending the CDP-JAK inhibitor conjugate in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the CDP-JAK inhibitor conjugate, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-JAK inhibitor conjugate may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more CDP-JAK inhibitor conjugate described herein with one or more suitable non-irritating excipients which is solid at room temperature, but liquid at body temperature. The composition will therefore melt in the rectum or vaginal cavity and release the CDP-JAK inhibitor conjugate. Such materials include, for example, cocoa butter, polyethylene glycol, a suppository wax or a salicylate. Compositions of the disclosure, which are suitable for vaginal administration, also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the disclosure.

### Subcutaneous Administration

In another aspect, the disclosure features subcutaneous administration of a CDP-JAK inhibitor conjugate, e.g., a CDP-JAK inhibitor conjugate described herein to a subject, e.g., a human subject. For such purposes, the CDP-JAK inhibitor conjugates, e.g., the CDP-JAK inhibitor conjugates described herein described can be formulated using methods known in the art of formulation chemistry, and may be injected, e.g., using a syringe, as well as other devices including injection devices (e.g., the Inject-ease® and Genject® devices); injector pens (such as the GenPen®); needleless devices (e.g., Medi-Jector and Biojector® 2000); and subcutaneous patch delivery systems. In some embodiments, the device, e.g., a syringe, e.g., an autoinjector pen, contains a needle with a gauge ranging in size from 25 G or smaller in diameter. In some embodiments, the needle gauge ranges in size from 25 G to 33 G (including ranges intermediate thereto, e.g., 25 sG, 26, 26 sG, 27 G, 28 G, 29 G, 30 G, 31 G, 32 G, and 33 G). In one embodiment, the smallest needle diameter and appropriate length is chosen in accordance with the viscosity characteristics of the formulation and the device used to deliver the formulation of the CDP-JAK inhibitor conjugate described herein.

Examples of needleless devices include, but are not limited to, Biojector® 2000 (Bioject Medical Technologies), Cool.Click™ (Bioject Medical Technologies), Iject™ (Bioject Medical Technologies), Vitajet™ 3, (Bioject Medical Technologies), Mhi500 (The Medical House PLC), Injex 30 (INJEX-Equidyne Systems), Injex 50 (INJEX-Equidyne Systems), Injex 100 (INJEX-Equidyne Systems), Jet Syringe (INJEX-Equidyne Systems), Jetinjector (Becton-Dickinson), J-Tip® (National Medical Devices, Inc.), Medi-Jector VISION® (Antares Pharma), MED-JET® (MIT Canada, Inc.), DermoJet®(Akra Dermojet), Sonoprep® (Sontra Medical Corp.), PenJet® (PenJet Corp.), MicroPor (Altea Therapeutics), Zeneo® (Crossject Medical Technology), Mini-Ject® (Valeritas Inc), ImplaJect® (Caretek Medical LTD), Intraject® (Aradigm), and Serojet® (Bioject Medical Technologies).

In one embodiment, the CDP-JAK inhibitor conjugate, e.g., the CDP-JAK inhibitor conjugate described herein, is formulated for subcutaneous administration. In one embodiment, the subcutaneous formulation comprising the CDP-JAK inhibitor conjugate is a sterile, preservative-free solution that includes the CDP-JAK inhibitor conjugate. In one embodiment, the disclosure features an article of manufacture, e.g., a device described herein (e.g., a syringe or injector pen for subcutaneous administration) that contains a subcutaneous formulation comprising a CDP-JAK inhibitor conjugate described herein. In one embodiment, the article of manufacture is a single-use, prefilled pen or as a single-use, prefilled glass syringe (e.g., a pen or syringe described herein. In one embodiment, the article of manufacture is filled with 1 mL of a subcutaneous formulation comprising the CDP-JAK inhibitor conjugate. In one embodiment, the subcutaneous formulation includes in an amount of CDP-JAK inhibitor conjugate such that 15 mg, 20 mg, 25, mg, 30 mg, 35 mg, 40 mg, 45 mg or 50 mg of the JAK inhibitor is present in the formulation.

Also included in the disclosure are delivery devices that house a formulation comprising a CDP-JAK inhibitor conjugate, e.g., a CDP-JAK inhibitor conjugate described herein. Examples of such devices include, but are not limited to, a syringe, a pen (such as an autoinjector pen), an implant, an inhalation device, a needleless device, and a patch. An example of an autoinjection pen is described in U.S. application Ser. No. 11/824,516, filed Jun. 29, 2007.

### Dosages and Dosage Regimens

The CDP-JAK inhibitor conjugate can be formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular subject, composition, and mode of administration, without being toxic to the subject.

In one embodiment, the CDP-JAK inhibitor conjugate is administered to a subject at a dosage of, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.13 mg/kg, 0.15 mg/kg, 0.18 mg/kg, 0.20 mg/kg, 0.23 mg/kg, 0.25 mg/kg, 0.28 mg/kg, 0.30 mg/kg, 0.33 mg/kg, 0.35 mg/kg, 0.38 mg/kg, 0.40 mg/kg, 0.43 mg/kg, 0.45 mg/kg, 0.48 mg/kg, 0.50 mg/kg of the JAK inhibitor. Administration can be at regular intervals, such as every 1, 2, 3, 4, or 5 days, or weekly, or every 2, 3, 4, 5, 6, or 7 or 8 weeks.

The administration, e.g., intravenous administration, can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the CDP-JAK inhibitor conjugate is administered as a bolus infusion or intravenous push, e.g., over a period of 15 minutes, 10 minutes, 5 minutes or less. In one embodiment, the CDP-JAK inhibitor is administered in an amount such the desired dose of the agent is administered. Preferably the dose of the CDP-JAK inhibitor conjugate is a dose described herein.

The administration, e.g., subcutaneous administration, can be administered via injection under the skin. In one embodiment, the CDP-JAK inhibitor is administered in an amount such the desired dose of the agent is administered. Preferably the dose of the CDP-JAK inhibitor conjugate is a dose described herein.

In one embodiment, the subject receives 1, 2, 3, up to 10 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The CDP-JAK inhibitor conjugate can be administered as a first line therapy, e.g., alone or in combination with an additional agent or agents. In other embodiments, a CDP-JAK inhibitor is administered after a subject has developed resistance to, has filed to respond to or has relapsed after a first line therapy. The CDP-JAK inhibitor conjugate can be administered in combination with a second agent. Preferably, the CDP-JAK inhibitor is administered in combination with a second agent described herein.

### Kits

A CDP-JAK inhibitor described herein may be provided in a kit. The kit includes a CDP-JAK inhibitor conjugate described herein and, optionally, a container, a pharmaceutically acceptable carrier and/or informational material. The informational material can be descriptive, instructional, marketing or other material that relates to the methods described herein and/or the use of the CDP-JAK inhibitor conjugate for the methods described herein.

The informational material of the kits is not limited in its form. In one embodiment, the informational material can include information about production of the CDP-JAK inhibitor conjugate, physical properties of the CDP-JAK inhibitor conjugate, concentration, date of expiration, batch or production site information, and so forth. In one embodiment, the informational material relates to methods for administering the CDP-JAK inhibitor.

In one embodiment, the informational material can include instructions to administer a CDP-JAK inhibitor conjugate described herein in a suitable manner to perform the methods described herein, e.g., in a suitable dose, dosage form, or mode of administration (e.g., a dose, dosage form, or mode of administration described herein). In another embodiment, the informational material can include instructions to administer a CDP-JAK inhibitor conjugate described herein to a suitable subject, e.g., a human, e.g., a human having or at risk for a disorder described herein. In another embodiment, the informational material can include instructions to reconstitute a CDP-JAK inhibitor conjugate described herein into a pharmaceutically acceptable composition.

In one embodiment, the kit includes instructions to use the CDP-JAK inhibitor conjugate, such as for treatment of a subject. The instructions can include methods for reconstituting or diluting the CDP-JAK inhibitor conjugate for use with a particular subject or in combination with a particular agent. The instructions can also include methods for reconstituting or diluting the CDP-JAK inhibitor conjugate for use with a particular means of administration, such as by intravenous infusion or subcutaneous administration.

In another embodiment, the kit includes instructions for treating a subject with a particular indication, such as a particular cancer, a cancer at a particular stage, a particular autoimmune disorder, or a particular inflammatory disorder. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid. In another embodiment, the instructions will describe treatment of selected subjects with the CDP-JAK inhibitor conjugate. For example, the instructions can describe treatment of one or more of: a subject who has received an anticancer agent (e.g., a JAK inhibitor) and has a neutrophil or platelet count less than a standard; a subject who has moderate to severe neutropenia; a subject who has thrombocytopenia; a subject having hepatic impairment, e.g., having transaminase (ALT and/or AST levels) greater than the upper limit of normal (ULN) and/or bilirubin levels greater than ULN; a subject having hepatic impairment, e.g., ALP levels greater than the upper limit of normal (ULN), SGOT and/or SGPT levels greater the upper limit of normal (ULN) and/or bilirubin levels greater than the ULN; a subject who has experienced or is at risk for renal impairment, a subject who has or is at risk of having a gastroinstinal perforation (e.g., associated with the administration of a chemotherapeutic agent (e.g., a JAK inhibitor)), and a subject who has or is at risk for having an infection.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a CDP-JAK inhibitor conjugate described herein and/or its use in the methods described herein. The informational material can also be provided in any combination of formats.

In addition to a CDP-JAK inhibitor conjugate described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a CDP-JAK inhibitor described herein. In such embodiments, the kit can include instructions for admixing a CDP-JAK inhibitor conjugate described herein and the other ingredients, or for using a CDP-JAK inhibitor conjugate described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-JAK inhibitor conjugate and the second therapeutic agent are in separate containers, and in another embodiment, the CDP-JAK inhibitor conjugate and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A CDP-JAK inhibitor described herein can be provided in any form, e.g., liquid, frozen, dried or lyophilized form. It is preferred that a particle described herein be substantially pure and/or sterile. When a CDP-JAK inhibitor conjugate described herein is provided in a liquid solution, the liquid solution preferably is an aqueous solution, with a sterile aqueous solution being preferred. In one embodiment, the CDP-JAK inhibitor conjugate is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a CDP-JAK inhibitor conjugate described herein. In some embodiments, the kit contains separate containers, dividers or compartments for the composition and informational material. For example, the composition can be contained in a bottle, vial, IV admixture bag, IV infusion set, piggyback set or syringe, as well as other devices including injection devices (e.g., the Inject-ease® and Genject® devices); injector pens (such as the GenPen®); needleless devices (e.g., Medi-Jector and Biojector® 2000); and subcutaneous patch delivery systems, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a CDP-JAK inhibitor conjugate described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Combination Therapy

The CDP-JAK inhibitor conjugate may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-JAK inhibitor conjugate and the at least one additional therapeutic agent can be administered simultaneously, in the same or in separate compositions, or sequentially. For sequential administration, the CDP-JAK inhibitor conjugate can be administered first, and the additional agent can be administered second, or the order of administration can be reversed.

In some embodiments, the CDP-JAK inhibitor conjugate is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

### Combination Therapy—Cancer

In some embodiments, the CDP-JAK inhibitor conjugate is administered with at least one additional therapeutic agent, such as a chemotherapeutic agent. In certain embodiments, the CDP-JAK inhibitor is administered in combination with one or more additional chemotherapeutic agent, e.g., with one or more chemotherapeutic agents described herein. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., CRLX101).

taxanes: paclitaxel (Taxol®), docetaxel (Taxotere®), larotaxel, cabazitaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

proteosome inhibitors: bortezomib (Velcade®).

interferons (e.g., IFN-alpha (Alferon®, Roferon-A®, Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens, which include without limitation, nilutamide (Nilandron®) and bicalutamide (Caxodex®).

antiestrogens, which include without limitation, tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents, which include without limitation, gallium (III) nitrate hydrate (Ganite®) and pamidronate disodium (Aredia®).

apoptosis inducers, which include without limitation, ethanol, 2-[[3-(2,3-dichlorophenoxy)propyl]amino]-(9Cl), gambogic acid, embelin and arsenic trioxide (Trisenox®).

Aurora kinase inhibitors, which include without limitation, binucleine 2.

Bruton's tyrosine kinase inhibitors, which include without limitation, terreic acid.

calcineurin inhibitors, which include without limitation, cypermethrin, deltamethrin, fenvalerate and tyrphostin 8.

CaM kinase II inhibitors, which include without limitation, 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors, which include without limitation, phosphonic acid.

CDC25 phosphatase inhibitors, which include without limitation, 1,4-naphthalene dione, 2,3-bis[(2-hydroxyethyl)thio]-(9Cl).

CHK kinase inhibitors, which include without limitation, debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors, which include without limitation, 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors, which include without limitation, olomoucine and its derivatives, purvalanol B, roascovitine (Seliciclib®), indirubin, kenpaullone, purvalanol A and indirubin-3′-monooxime.

cysteine protease inhibitors, which include without limitation, 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators, which include without limitation, plicamycin (Mithracin®) and daptomycin (Cubicin®).

DNA strand breakers, which include without limitation, bleomycin (Blenoxane®).

E3 ligase inhibitors, which include without limitation, N-((3,3,3-trifluoro-2-trifluoromethyl)propionyl)sulfanilamide.

EGF Pathway Inhibitors, which include without limitation, tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors, which include without limitation, A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors, which include without limitation, 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors, which include without limitation, indirubin-3′-monooxime

histone deacetylase (HDAC) inhibitors, which include without limitation, suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK), which include without limitation, 2-propenenitrile, 3-[(4-methylphenyl)sulfonyl]-(2E)-(9Cl).

imidazotetrazinones, which include without limitation, temozolomide (Methazolastone®, Temodar® and its derivatives (e.g., as disclosed generically and specifically in U.S. Pat. No. 5,260,291) and Mitozolomide.

insulin tyrosine kinase inhibitors, which include without limitation, hydroxyl-2-naphthalenylmethylphosphonic acid.

c-Jun-N-terminal kinase (JNK) inhibitors, which include without limitation, pyrazoleanthrone and epigallocatechin gallate.

mitogen-activated protein kinase (MAP) inhibitors, which include without limitation, benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors, which include without limitation, trans-4-iodo, 4′-boranyl-chalcone.

MEK inhibitors, which include without limitation, butanedinitrile, bis[amino[2-aminophenyl)thio]methylene]-(9Cl).

MMP inhibitors, which include without limitation, Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors, which include without limitation, rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors, which include without limitation, tyrphostin AG 879.

p38 MAP kinase inhibitors, which include without limitation, Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors, which include without limitation, damnacanthal and tyrphostin 46.

PDGF pathway inhibitors, which include without limitation, tyrphostin AG 1296, tyrphostin 9,1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors, which include without limitation, wortmannin, and quercetin dihydrate.

phosphatase inhibitors, which include without limitation, cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors, which include without limitation, rottlerin.

polyamine synthesis inhibitors, which include without limitation, DMFO.

proteasome inhibitors, which include without limitation, aclacinomycin A, gliotoxin and bortezomib (Velcade®).

PTP1B inhibitors, which include without limitation, L-leucinamide.

protein tyrosine kinase inhibitors, which include without limitation, tyrphostin Ag 216, tyrphostin Ag 1288, tyrphostin Ag 1295, geldanamycin, genistein and 7H-pyrollo[2,3-d]pyrimidine derivatives of formula I as generically and specifically described in PCT Publication No.: WO 03/013541 and U.S. Publication No.: 2008/0139587:

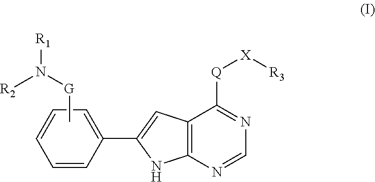

Publication No.: 2008/0139587 discloses the various substituents, e.g., R₁, R₂, etc.

SRC family tyrosine kinase inhibitors, which include without limitation, PP1 and PP2.

Syk tyrosine kinase inhibitors, which include without limitation, piceatannol.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors, which include without limitation, 2-aminopurine.

sterol biosynthesis inhibitors, which include without limitation, squalene epoxidase and CYP2D6.

VEGF pathway inhibitors, which include without limitation, anti-VEGF antibodies, e.g., bevacizumab, and small molecules, e.g., sunitinib (Sutent®), sorafinib (Nexavar®), ZD6474 (also known as vandetanib) (Zactima™, SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiment, the CDP-JAK inhibitor conjugate is administered instead of another tyrosine kinase inhibitor, e.g., a free JAK inhibitor, as a first line therapy or a second line therapy.

In some cases, a hormone and/or steriod can be administered in combination with a CDP-JAK inhibitor conjugate. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-JAK inhibitor conjugate is administered in combination with an anti-microbial (e.g., leptomycin B).

In another embodiment, the CDP-JAK inhibitor conjugate is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the disclosure are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-JAK inhibitor conjugate is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a CDP-JAK inhibitor conjugate is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

When formulating the pharmaceutical compositions featured in the disclosure the clinician may utilize preferred dosages as warranted by the condition of the subject being treated. For example, in one embodiment, a CDP-JAK inhibitor conjugate may be administered at a dosing schedule described herein, e.g., once every one, two three four, five, or six weeks.

Also, in general, a CDP-JAK inhibitor conjugate and an additional chemotherapeutic agent(s) do not have to be administered in the same pharmaceutical composition, and may, because of different physical and chemical characteristics, have to be administered by different routes. For example, the CDP-JAK inhibitor conjugate may be administered intravenously or subcutaneously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a CDP-JAK inhibitor conjugate is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, SN-38, camptothecin (e.g., CRLX101)); and a platinum-based agent (e.g., cisplatin (Platinol®), carboplatin (Paraplat®, Paraplatin®), oxaliplatin (Eloxatin®)).

In another embodiment, the CDP-JAK inhibitor conjugate is administered once every two weeks in combination with one or more additional chemotherapeutic agent that is administered orally. For example, the CDP-JAK inhibitor conjugate can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The disclosure also encompasses a method for the synergistic treatment of cancer wherein a CDP-JAK inhibitor conjugate is administered in combination with an additional chemotherapeutic agent or agents.

The particular choice of conjugate and anti-proliferative cytotoxic agent(s) or radiation will depend upon the diagnosis of the attending physicians and their judgment of the condition of the subject and the appropriate treatment protocol.

If the CDP-JAK inhibitor conjugate and the chemotherapeutic agent(s) and/or radiation are not administered simultaneously or essentially simultaneously, then the initial order of administration of the CDP-JAK inhibitor conjugate, and the chemotherapeutic agent(s) and/or radiation, may be varied. Thus, for example, the CDP-JAK inhibitor conjugate may be administered first followed by the administration of the chemotherapeutic agent(s) and/or radiation; or the chemotherapeutic agent(s) and/or radiation may be administered first followed by the administration of the CDP-JAK inhibitor conjugate. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-JAK inhibitor conjugate, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### Combination Therapy—Inflammation

In certain embodiments, a CDP-JAK inhibitor conjugate described herein may be administered alone or in combination with other compounds useful for treating or preventing inflammation. Exemplary anti-inflammatory agents include, for example, steroids (e.g., Cortisol, cortisone, fludrocortisone, prednisone, 6[alpha]-methylprednisone, triamcinolone, betamethasone or dexamethasone), nonsteroidal anti-inflammatory drugs (NSAIDS (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine) In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, [alpha]-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, caiprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide and cyclazocine.

Further examples of anti-inflammatory agents include deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocoitolone, fluoresone, fluorometholone, fluperolone, flupirtine, fluprednidene, fluprednisolone, fluproquazone, flurandrenolide, flurbiprofen, fluticasone, formocortal and fosfosal.

Further examples of anti-inflammatory agents include gentisic acid, glafenine, glucametacin, glycol salicylate, guaiazulene, halcinonide, halobetasol, halometasone, haloprednone, heroin, hydrocodone, hydro cortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate and myrophine.

Further examples of anti-inflammatory agents include nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide,norlevorphanol, normethadone, normorphine, norpipanone, olsalazine, opium, oxaceprol, oxametacine, oxaprozin, oxycodone, oxymorphone, oxyphenbutazone, papavereturn, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In one embodiment, a CDP-JAK inhibitor conjugate described herein may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, and lumiracoxib.

### Combination Therapy—Cardiovascular

In one embodiment, a CDP-JAK inhibitor conjugate described herein may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a CDP-JAK inhibitor conjugate described herein may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocamide, procainamide, encamide, flecanide, tocamide, phenyloin, propafenone, quinidine, disopyramide, and flecamide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltiazem, digitalis, adenosine, nickel chloride, and magnesium ions.

In another embodiment, a CDP-JAK inhibitor conjugate described herein may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; antiarrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex, or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this disclosure within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, fiunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin El and prostaglandin 12), an endothelin receptor blocking drug (such as bosentan), diltiazem, nicorandil, and nitroglycerin. Examples of cerebral protecting drugs include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of brain metabolic stimulants include amantadine, tiapride, and gamma-aminobutyric acid. Examples of anticoagulants include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of antiplatelet drugs include ticlopidine hydrochloride, dipyridamole, cilostazol, ethyl icosapentate, sarpogrelate hydrochloride, dilazep hydrochloride, trapidil, a nonsteroidal anti-inflammatory agent (such as aspirin), beraprostsodium, iloprost, and indobufene.

Examples of thrombolytic drugs include urokinase, tissue-type plasminogen activators (such as alteplase, tisokinase, nateplase, pamiteplase, monteplase, and rateplase), and nasaruplase. Examples of antihypertensive drugs include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of antianginal drugs include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline) trimetazidine, dipyridamole, etafenone, dilazep, trapidil, nicorandil, enoxaparin, and aspirin.

Examples of diuretics include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, andpotassiumcanrenoate), osmotic diuretics (such as isosorbide, D-mannitol, and glycerin), nonthiazide diuretics (such as meticrane, tripamide, chlorthalidone, and mefruside), and acetazolamide.

Examples of cardiotonics include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate. Examples of antiarrhythmic drugs include ajmaline, pirmenol, procainamide, cibenzoline, disopyramide, quinidine, aprindine, mexiletine, lidocaine, phenyloin, pilsicamide, propafenone, flecamide, atenolol, acebutolol, sotalol, propranolol, metoprolol, pindolol, amiodarone, nifekalant, diltiazem, bepridil, and verapamil Examples of antihyperlipidemic drugs include atorvastatin, simvastatin, pravastatin sodium, fluvastatin sodium, clinofibrate, clofibrate, simfibrate, fenofibrate, bezafibrate, colestimide, and colestyramine

Yet other exemplary cardiovascular agents include, for example, anti-angiogenic agents and vascular disrupting agents.

### Combination Therapy—Metabolic Disorder

In certain embodiments, a CDP-JAK inhibitor conjugate described herein may be administered alone or in combination with other compounds useful for treating or preventing a metabolic disorder, e.g., diabetes. Exemplary agents include, for example, alpha-glucosidase inhibitors such as miglitol (Glyset®), acarbose (Precose®); amylin analogs such as pramlintide (Symlin®); dipeptidyl peptidase 4 inhibitors such as sitagliptin (Januvia®), saxagliptin (Onglyza®), tolbutamide (Orinase®), linagliptin (Tradjenta®); insulin such as insulin glulisine (Apidra®, Apidra Solostar®), insulin glargine (Lantus®, Lantus Solostar®), insulin lispro (Humalog®, Humalog KwikPen®), insulin zinc (Humulin L®, Humulin U®, Iletin Lente®, Lente Iletin II®), Novolin LC)), insulin detemir (Levemir®), insulin aspart (Novolog®), insulin isophane (Humulin N®, Humulin N Pen®, Novolin N®, Relion Novolin N®), insulin (Exubera®, Humulin R®, Novolin R®, ReliOn/Novolin R®, Velosulin BR®); incretin mimetics such as exenatide (Bydureon®, Byetta®), liraglutide (Victoza®); meglitinides such as repaglinide (Prandin®), nateglinide (Starlix®), sulfonylureas such as glimepiride (Amaryl®), glyburide (DiaBeta®, Glycron®, Glynase®, Glynase PresTab®, Micronase®), chlorpropamide (Diabinese®), acetohexamide (Dymelor®), glipizide (GlipiZIDE XL®, Glucotrol®, Glucotrol XL®), tolbutamide (Tol-Tab®, Tolinase®); non-sulfonylureas such as metformin (Fortamet®, Glucophage®, Glucophage XR®, Glumetza®, Riomet®); thiazolidinediones such as pioglitazone (Actos®), rosiglitazone (Avandia®), troglitazone (Rezulin®), minerals and electrolytes such as chromium picolinate (Cr-GTF®, CRM®); and antidiabetic combinations such as metformin/pioglitazone (ActoPlus Met®, ActoPlus Met XR®); metformin/rosiglitazone (Avandamet®, Avandaryl®), metformin/saxagliptin (Kombiglyze XR®), glimepiride/pioglitazone (Duetact®), glyburide/metformin (Glucovance®), metformin/sitagliptin (Janumet®), simvastatin/sitagliptin (Juvisync®), glipizide/metformin (Metaglip®), metformin/repaglinide (PrandiMet®).

The actual dosage of the CDP-JAK inhibitor conjugate and/or any additional therapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a CDP-JAK inhibitor conjugate is administered in combination with one or more additional therapeutic agents, the additional therapeutic agent (or agents) is administered at a standard dose.

In some embodiments, when a CDP-JAK inhibitor conjugate is administered in combination with one or more additional chemotherapeutic agents, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml×min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

### Methods

The CDP-JAK inhibitor conjugates described herein can decrease the activity of one or more Janus kinases (JAKs). Accordingly, the CDP-JAK inhibitor conjugates can be used in methods of modulating a JAK by contacting the JAK with any one or more of the CDP-JAK inhibitor conjugates described herein.

JAKs to which the CDP-JAK inhibitor conjugates described herein bind and/or modulate include any member of the JAK family. In some embodiments, the JAK is JAK1, JAK2, JAK3 or TYK2. In some embodiments, the JAK is JAK1 or JAK2. In some embodiments, the JAK is JAK2. In some embodiments, the JAK is JAK3.

The CDP-JAK inhibitor conjugates described herein can be selective. By “selective” is meant that the CDP-JAK inhibitor conjugates described herein bind to or inhibit a JAK with greater affinity or potency, respectively, compared to at least one other JAK. In some embodiments, the CDP-JAK inhibitor conjugates described herein are selective inhibitors of JAK1 or JAK2 over JAK3 and/or TYK2. In some embodiments, the CDP-JAK inhibitor conjugates described herein are selective inhibitors of JAK2 (e.g., over JAK1, JAK3 and TYK2). Without wishing to be bound by theory, because inhibitors of JAK3 can lead to immunosuppressive effects, a CDP-JAK inhibitor conjugate which is selective for JAK2 over JAK3 and which is useful in the treatment of cancer, e.g., a cancer described herein, can offer the additional advantage of having fewer immunosuppressive side effects. Selectivity can be at least about 5-fold, 10-fold, at least about 20-fold, at least about 50-fold, at least about 100-fold, at least about 200-fold, at least about 500-fold, or at least about 1000-fold. Selectivity can be measured by methods routine in the art. In some embodiments, selectivity can be tested at the Kₘ of each enzyme. In some embodiments, selectivity of the CDP-JAK inhibitor conjugates described herein for JAK2 over JAK3 can be determined by the cellular ATP concentration.

The CDP-JAK inhibitor conjugates described herein are useful in evaluating or treating proliferative disorders, e.g., treating a tumor and metastases thereof wherein the tumor or metastases thereof is a cancer described herein. The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer, childhood; esophageal cancer; esophageal cancer, childhood; gallbladder cancer; gastric (stomach) cancer; gastric (stomach) cancer, childhood; hepatocellular (liver) cancer, adult (primary); hepatocellular (liver) cancer, childhood (primary); extrahepatic; pancreatic cancer; pancreatic cancer, childhood; sarcoma, rhabdomyosarcoma; pancreatic cancer, islet cell; rectal cancer; and small intestine cancer;

Endocrine cancers such as islet cell carcinoma (endocrine pancreas); adrenocortical carcinoma; adrenocortical carcinoma, childhood; gastrointestinal carcinoid tumor; parathyroid cancer; pheochromocytoma; pituitary tumor; thyroid cancer; thyroid cancer, childhood; multiple endocrine neoplasia syndrome, childhood; and carcinoid tumor, childhood;

Eye cancers such as intraocular melanoma; and retinoblastoma;

Musculoskeletal cancers such as Ewing's family of tumors; osteosarcoma/malignant fibrous histiocytoma of the bone; rhabdomyosarcoma, childhood; soft tissue sarcoma, adult; soft tissue sarcoma, childhood; clear cell sarcoma of tendon sheaths; and uterine sarcoma;

Breast cancer such as breast cancer and pregnancy; breast cancer, childhood; and breast cancer, male;

Neurologic cancers such as brain stem glioma, childhood; brain tumor, adult; brain stem glioma, childhood; cerebellar astrocytoma, childhood; cerebral astrocytoma/malignant glioma, childhood; ependymoma, childhood; medulloblastoma, childhood; pineal and supratentorial primitive neuroectodermal tumors, childhood; visual pathway and hypothalamic glioma, childhood; other childhood brain cancers; adrenocortical carcinoma; central nervous system lymphoma, primary; cerebellar astrocytoma, childhood; neuroblastoma; craniopharyngioma; spinal cord tumors; central nervous system atypical teratoid/rhabdoid tumor; central nervous system embryonal tumors; andsupratentorial primitive neuroectodermal tumors, childhood and pituitary tumor;

Genitourinary cancers such as bladder cancer; bladder cancer, childhood; kidney cancer; ovarian cancer, childhood; ovarian epithelial cancer; ovarian low malignant potential tumor; penile cancer; prostate cancer; renal cell cancer, childhood; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as extracranial germ cell tumor, childhood; extragonadal germ cell tumor; ovarian germ cell tumor; and testicular cancer;

Head and neck cancers such as lip and oral cavity cancer; oral cancer, childhood; hypopharyngeal cancer; laryngeal cancer; laryngeal cancer, childhood; metastatic squamous neck cancer with occult primary; mouth cancer; nasal cavity and paranasal sinus cancer; nasopharyngeal cancer; nasopharyngeal cancer, childhood; oropharyngeal cancer; parathyroid cancer; pharyngeal cancer; salivary gland cancer; salivary gland cancer, childhood; throat cancer; and thyroid cancer;

Hematologic/blood cell cancers such as a leukemia (e.g., acute lymphoblastic leukemia, adult; acute lymphoblastic leukemia, childhood; acute myeloid leukemia, adult; acute myeloid leukemia, childhood; chronic lymphocytic leukemia; chronic myelogenous leukemia; and hairy cell leukemia); a lymphoma (e.g., AIDS-related lymphoma; cutaneous T-cell lymphoma; Hodgkin's lymphoma, adult; Hodgkin's lymphoma, childhood; Hodgkin's lymphoma during pregnancy; non-Hodgkin's lymphoma, adult; non-Hodgkin's lymphoma, childhood; non-Hodgkin's lymphoma during pregnancy; mycosis fungoides; sezary syndrome; T-cell lymphoma, cutaneous; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; thymoma, childhood; thymic carcinoma; bronchial adenomas/carcinoids; pleuropulmonary blastoma; non-small cell lung cancer; and small cell lung cancer;

Skin cancers such as Kaposi's sarcoma; Merkel cell carcinoma; melanoma; and skin cancer, childhood;

Other childhood cancers and cancers of unknown primary site;

and metastases of the aforementioned cancers can also be treated or prevented in accordance with the methods described herein.

The CDP-JAK inhibitor conjugates described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In further embodiments, the proliferative disorder is a JAK-associated cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma etc.), hematological cancers (e.g., lymphoma, leukemia such as acute lymphoblastic leukemia, or multiple myeloma), cutaneous T-cell lymphoma (CTCL) and cutaneous B-cell lymphoma. Example cutaneous T-cell lymphomas include Sezary syndrome and mycosis fungoides.

In further embodiments, the proliferative disorder is a JAK-associated cancer including myeloproliferative disorders (MPDs) such as polycythemia vera (PV), essential thrombocythemia (ET), myeloid metaplasia with myelofibrosis (MMM), chronic myelogenous leukemia (CML), chronic myelomonocytic leukemia (CMML), hypereosinophilic syndrome (HES), systemic mast cell disease (SMCD), and the like.

In one embodiment, a method is provided for a combination treatment of a cancer, such as by treatment with a CDP-JAK inhibitor conjugate and a second therapeutic agent. Various combinations are described herein. The combination can reduce the development of tumors, reduces tumor burden, or produce tumor regression in a mammalian host.

In some embodiments, the proliferative disorder is a disease or disorder associated with inflammation. A CDP-JAK inhibitor conjugates described herein may be administered prior to the onset of, at, or after the initiation of inflammation. When used prophylactically, the CDP-JAK inhibitor conjugates described herein are preferably provided in advance of any inflammatory response or symptom. Administration of the CDP-JAK inhibitor conjugates described herein may prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In some embodiments, the inflammatory disorder is a JAK-associated inflammatory diseases. Example inflammatory diseases include inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis, and other inflammatory diseases.

In some embodiments, the CDP-JAK inhibitor conjugates described herein can be useful in treating a disease or disorder such as a cardiovascular disease as described herein. Examples of cardiovascular diseases include, but are not limited to: angina; arrhythmias (atrial or ventricular or both), or long-standing heart failure; arteriosclerosis; atheroma; atherosclerosis; cardiac hypertrophy including both atrial and ventricular hypertrophy; cardiac or vascular aneurysm; cardiac myocyte dysfunction; carotid obstructive disease; congestive heart failure; endothelial damage after PTCA (percutaneous transluminal coronary angioplasty); hypertension including essential hypertension, pulmonary hypertension and secondary hypertension (renovascular hypertension, chronic glomerulonephritis); myocardial infarction; myocardial ischemia; peripheral obstructive arteriopathy of a limb, an organ, or a tissue; peripheral artery occlusive disease (PAOD); reperfusion injury following ischemia of the brain, heart or other organ or tissue; restenosis; stroke; thrombosis; transient ischemic attack (TIA); vascular occlusion; vasculitis; and vasoconstriction.

In one embodiment, the cardiovascular disease can be an inflammatory disease of the heart such as cardiomyopathy, ischemic heart disease, hypercholesterolemia, and atherosclerosis.

In some embodiments, the CDP-JAK inhibitor conjugates described herein can be useful in treating a disease or disorder such as an autoimmune disease as described herein. Examples of autoimmune diseases include, but are not limited to: acute disseminated encephalomyelitis (ADEM); Addison's disease; antiphospholipid antibody syndrome (APS); aplastic anemia; autoimmune hepatitis; cancer; coeliac disease; Crohn's disease; Diabetes mellitus (type 1); Goodpasture's syndrome; Graves' disease; Guillain-Barre syndrome (GBS); Hashimoto's disease; lupus erythematosus; multiple sclerosis; myasthenia gravis; opsoclonus myoclonus syndrome (OMS); optic neuritis; Ord's thyroiditis; oemphigus; polyarthritis; primary biliary cirrhosis; psoriasis; rheumatoid arthritis; Reiter's syndrome; Takayasu's arteritis; temporal arteritis (also known as “giant cell arteritis”); warm autoimmune hemolytic anemia; Wegener's granulomatosis; alopecia universalis; Chagas disease; chronic fatigue syndrome; dysautonomia; endometriosis; hidradenitis suppurativa; interstitial cystitis; neuromyotonia; sarcoidosis; scleroderma; ulcerative colitis; vitiligo; and vulvodynia.

In some embodiments, the CDP-JAK inhibitor conjugates described herein can be useful in treating a disease or disorder such as an inflammatory disease as described herein. Examples of inflammatory disease include, but are not limited to: inflammation associated with acne; anemia (e.g., aplastic anemia, haemolytic autoimmune anaemia); asthma; arteritis (e.g., polyarteritis, temporal arteritis, periarteritis nodosa, Takayasu's arteritis); arthritis (e.g., crystalline arthritis, osteoarthritis, psoriatic arthritis, gouty arthritis, reactive arthritis, rheumatoid arthritis and Reiter's arthritis); ankylo sing spondylitis; amylosis; amyotrophic lateral sclerosis; allergies or allergic reactions; Alzheimer's disease; atherosclerosis; bronchitis; bursitis; chronic prostatitis; conjunctivitis; Chagas disease; chronic obstructive pulmonary disease; cermatomyositis; diverticulitis; diabetes (e.g., type I diabetes mellitus, type 2 diabetes mellitus); dermatitis; eosinophilic gastrointestinal disorders (e.g., eosinophilic esophagitis, eosinophilic gastritis, eosinophilic gastroenteritis, eosinophilic colitis); eczema; endometriosis; gastrointestinal bleeding; gastritis; gastroesophageal reflux disease (GORD, or its synonym GERD); Guillain-Barre syndrome; infection; ischaemic heart disease; Kawasaki disease; glomerulonephritis; gingivitis; hypersensitivity; headaches (e.g., migraine headaches, tension headaches); ileus (e.g., postoperative ileus and ileus during sepsis); idiopathic thrombocytopenic purpura; interstitial cystitis; inflammatory bowel disease (IBD) (e.g., Crohn's disease, ulcerative colitis, collagenous colitis, lymphocytic colitis, ischaemic colitis, diversion colitis, Behcet's syndrome, indeterminate colitis); inflammatory bowel syndrome (IBS); lupus; multiple sclerosis; morphea; myeasthenia gravis; myocardial ischemia; nephrotic syndrome; pemphigus vulgaris; pernicious anemia; peptic ulcers; psoriasis; polymyositis; primary biliary cirrhosis; Parkinson's disease; pelvic inflammatory disease; reperfusion injury; regional enteritis; rheumatic fever; systemic lupus erythematosus; schleroderma; scierodoma; sarcoidosis; spondyloarthopathies; Sjogren's syndrome; thyroiditis; transplantation rejection; tendonitis; trauma or injury (e.g., frostbite, chemical irritants, toxins, scarring, burns, physical injury); vasculitis; vitiligo; and Wegener's granulomatosis.

Examples of JAK-associated diseases include diseases involving the immune system including, for example, organ transplant rejection (e.g., allograft rejection and graft versus host disease).

Further examples of JAK-associated diseases include autoimmune diseases such as multiple sclerosis, rheumatoid arthritis, juvenile arthritis, type I diabetes, lupus, psoriasis, inflammatory bowel disease, ulcerative colitis, Crohn's disease, myasthenia gravis, immunoglobulin nephropathies, autoimmune thyroid disorders, and the like. In some embodiments, the autoimmune disease is an autoimmune bullous skin disorder such as pemphigus vulgaris (PV) or bullous pemphigoid (BP).

Further examples of JAK-associated diseases include allergic conditions such as asthma, food allergies, atopic dermatitis and rhinitis. Further examples of JAK-associated diseases include viral diseases such as Epstein Barr Virus (EBV), Hepatitis B, Hepatitis C, HIV, HTLV 1, Varicella-Zoster Virus (VZV) and Human Papilloma Virus (HPV).

Further examples of JAK-associated diseases or conditions include skin disorders such as psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, co-administration or sequential administration of at least one CDP-JAK inhibitor conjugate described herein together with the agent causing unwanted sensitization can be helpful in treating such unwanted sensitization or dermatitis. In some embodiments, the skin disorder is treated by topical administration of at least one CDP-JAK inhibitor conjugate described herein.

In some embodiments, the CDP-JAK inhibitor conjugates described herein can be useful in treating a disease or disorder such as a metabolic disorder. As described herein, the term “metabolic disorder” includes a disorder, disease or condition which is caused or characterized by an abnormal metabolism (i.e., the chemical changes in living cells by which energy is provided for vital processes and activities) in a subject. Examples of disorders include obesity, diabetes, a co-morbidity of obesity, and an obesity related disorder. The subject to whom the polymer-agent, particle or composition is administered may be overweight or obese. Alternatively, or in addition, the subject may be diabetic, for example having insulin resistance or glucose intolerance, or both. The subject may have diabetes mellitus, for example, the subject may have Type II diabetes. The subject may be overweight or obese and have diabetes mellitus, for example, Type II diabetes.

In addition, or alternatively, the subject may have, or may be at risk of having, a disorder in which obesity or being overweight is a risk factor. As used herein, “obesity” refers to a body mass index (BMI) of 30 kg/m² or more (National Institute of Health, Clinical Guidelines on the Identification, Evaluation, and Treatment of Overweight and Obesity in Adults (1998)). However, the disclosure is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, all of which are typically referred to as overweight (National Institute of Health, Clinical Guidelines on the Identification, Evaluation, and Treatment of Overweight and Obesity in Adults (1998)). Such disorders include, but are not limited to, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension; hypercholesterolemia; cholithiasis; osteoarthritis; orthopedic injury; insulin resistance, for example, type 2 diabetes and syndrome X; metabolic syndrome; and thromboembolic disease (see Kopelman (2000), Nature 404:635-43; Rissanen et al., British Med. J. 301, 835, 1990).

Other disorders associated with obesity include, but are not limited to, depression, anxiety, panic attacks, migraine headaches, PMS, chronic pain states, fibromyalgia, insomnia, impulsivity, obsessive-compulsive disorder, irritable bowel syndrome (IBS), and myoclonus. Furthermore, obesity is a recognized risk factor for increased incidence of complications of general anesthesia. (See e.g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatititis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odors and infections in the skin folds under the breasts, etc.; large anterior abdominal wall masses, for example abdominal panniculitis with frequent panniculitis, impeding walking, causing frequent infections, odors, clothing difficulties, lower back pain; musculoskeletal disease; pseudo tumor cerebri (or benign intracranial hypertension), and sliding hiatil hernia.

Conditions or disorders associated with increased caloric intake include, but are not limited to, insulin resistance, glucose intolerance, obesity, diabetes, including type 2 diabetes, eating disorders, insulin-resistance syndromes, metabolic syndrome X, and Alzheimer's disease.

In some embodiments, the CDP-JAK inhibitor conjugates described herein can be useful in treating a disease or disorder such as a central nervous system (CNS) disorder. Examples of central nervous system disorders include, but are not limited to: a myelopathy; an encephalopathy; central nervous system (CNS) infection; encephalitis (e.g., viral encephalitis, bacterial encephalitis, parasitic encephalitis); meningitis (e.g., spinal meningitis, bacterial meningitis, viral meningitis, fungal meningitis); neurodegenerative diseases (e.g., Huntington's disease; Alzheimer's disease; Parkinson's disease; multiple sclerosis; amyotrophic lateral sclerosis; traumatic brain injury); mental health disorder (e.g., schizophrenia, depression, dementia); pain and addiction disorders; brain tumors (e.g., intra-axial tumors, extra-axial tumors); adult brain tumors (e.g., glioma, glioblastoma); pediatric brain tumors (e.g., medulloblastoma); cognitive impairment; genetic disorders (e.g., Huntington's disease, neurofibromatosis type 1, neurofibromatosis type 2, Tay-Sachs disease, tuberous sclerosis); headache (e.g., tension headache; migraine headache, cluster headache, meningitis headache, cerebral aneurysm and subarachnoid hemorrhage headache, brain tumor headache); stroke (e.g., cerebral ischemia or cerebral infarction, transient ischemic attack, hemorrhagic (e.g., aneurysmal subarachnoid hemorrhage, hypertensive hemorrhage, other sudden hemorrhage)); epilepsy; spinal disease (e.g., degenerative spinal disease (e.g., herniated disc disease, spinal stenosis, and spinal instability), traumatic spine disease; spinal cord trauma; spinal tumors; hydrocephalus (e.g., communicating or non-obstructive hydrocephalus, non-communicating or obstructive hydrocephalus, adult hydrocephalus, pediatric hydrocephalus, normal pressure hydrocephalus, aqueductal stenosis, tumor associated hydrocephalus, pseudotumor cerebri); CNS vasculitis (e.g., primary angiitis of the central nervous system, benign angiopathy of the central nervous system; Arnold Chiari malformation; neuroAIDS; retinal disorders (e.g., age-related macular degeneration, wet age-related macular degeneration, myopic macular degeneration, retinitis pigmentosa, proliferative retinopathies); inner ear disorders; tropical spastic paraparesis; arachnoid cysts; locked-in syndrome; Tourette's syndrome; adhesive arachnoiditis; altered consciousness; autonomic neuropathy; benign essential tremor; brain anomalies; cauda equine syndrome with neurogenic bladder; cerebral edema; cerebral spasticity; cerebral vascular disorder; and Guillain-Barre syndrome.

In some embodiments, the CDP-JAK inhibitor conjugates described herein can be useful in treating a disease or disorder such as neurological deficits. As used herein, the phrase “neurological deficits” includes an impairment or absence of a normal neurological function or presence of an abnormal neurological function. Neurodegeneration of the brain can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

The CDP-JAK inhibitor conjugates described herein can be administered to a subject undergoing or who has undergone angioplasty. In one embodiment, the CDP-JAK inhibitor conjugates described herein can be administered to a subject undergoing or who has undergone angioplasty with a stent placement. In some embodiments, the CDP-JAK inhibitor conjugates described herein can be used as a strut of a stent or a coating for a stent.

The CDP-JAK inhibitor conjugates described herein can be used during the implantation of a stent, e.g., as a separate intravenous administration, as coating for a stent or as the strut of a stent.

Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The cyclodextrin containing polymer used in the following examples was of the following structure:

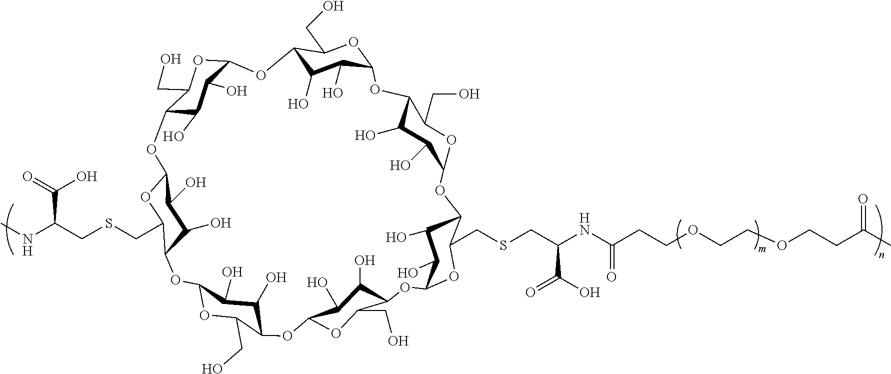

wherein the group

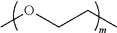

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)) and n is at least 4, 5, 6, 7, 8, 9, 10, 11, 12, 13, 14, 15, 16, 17, 18, 19 or 20.

### Example 1

### Synthesis of Ethylene Glycol Monoglycinate

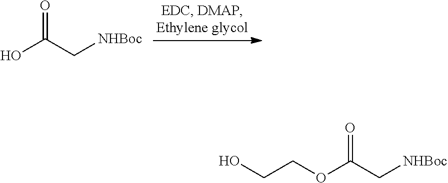 [SMILES: CC(C)(C)OC(=O)NCC(=O)O ; CC(C)(C)OC(=O)NCC(=O)OCCO]

A 300 mL round-bottom flask equipped with a magnetic stirrer was charged with N-Boc-glycine (2.547 g, 14.5 mmol), ethylene glycol (4.512 g, 72.7 mmol), N,N-dimethylaminopyridine (248 mg, 2.0 mmol) and dichloromethane (100 mL). The mixture was cooled in an ice bath and stirred for 5 min to produce a clear solution, to which 1-ethyl-3-(3-dimethyllaminopropyl)carbodiimide hydrochloride (EDC.HCl) (3.612 g, 18.9 mmol) was added. The mixture was stirred at ambient temperature for 18 h and then the reaction was diluted with DCM (200 mL) and washed with 1N HCl (150 mL), distilled water (150 mL) and brine (150 mL). The organic layer was separated, dried over sodium sulfate, and filtered. The filtrate was concentrated to a residue and loaded directly onto a 40-g silica column (Teledyne Isco). The product was eluted from the silica using a 0-100% gradient of ethyl acetate in hexane. The product was vacuum-dried at ambient temperature to afford the product, ethylene glycol monoglycinate as a clear oil [1.77 g, yield: 56%]. The ¹H NMR analysis was consistent with the assigned structure of the desired product.

### Example 2

### Synthesis of CDI Activated Ethylene Glycol Monoglycinate

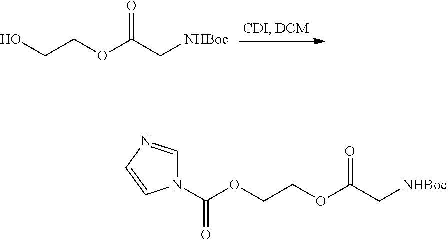 [SMILES: CC(C)(C)OC(=O)NCC(=O)OCCO ; CC(C)(C)OC(=O)NCC(=O)OCCOC(=O)n1ccnc1]

A 100 mL round-bottom flask equipped with a magnetic stir bar was charged with ethylene glycol monoglycinate (1.77 g, 8.07 mmol) and dichloromethane (20 mL). The mixture was stirred for 5 minutes to produce a clear solution, to which carbonyl diimidazole (2.618 g, 16.15 mmol). The reaction stirred at ambient temperature for 18 hours and then diluted with DCM (100 mL). The organic layer was washed with distilled water (2×100 mL) and brine (1×100 mL) and then concentrated under reduced pressure to afford the product as a white solid [2.40 g, yield: 95%]. The ¹H NMR analysis was consistent with the assigned structure of the desired product.

### Example 3

### Synthesis of Tofacitinib Ethylene Glycol Monoglycinate

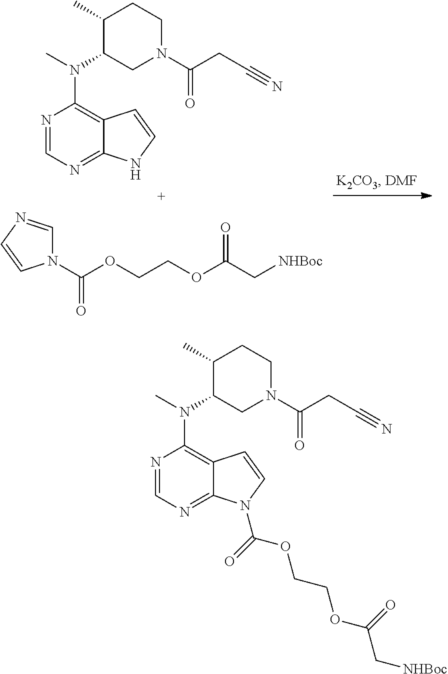 [SMILES: C[C@@H]1CCN(C(=O)CC#N)C[C@@H]1N(C)c1ncnc2[nH]ccc12 ; CC(C)(C)OC(=O)NCC(=O)OCCOC(=O)n1ccnc1 ; C[C@@H]1CCN(C(=O)CC#N)C[C@@H]1N(C)c1ncnc2c1ccn2C(=O)OCCOC(=O)CNC(=O)OC(C)(C)C]

A 20 mL reaction vial equipped with a magnetic stir bar was charged with Tofacitinib (1.041 g, 3.33 mmol), and carbonyl diimidazole activated ethylene glycol monoglycinate (1.043 mg, 3.33 mmol), and anhydrous dimethylformamide (10 mL). The mixture was stirred for 5 minutes to produce a clear solution, to which anhydrous potassium carbonate (1.378 mg, 10.0 mmol) was added. The mixture stirred at ambient temperature overnight and was then diluted with DCM (200 mL). The organic layer was washed with 1N HCl (1×00 mL), distilled water (2×100 mL), and brine (1×100 mL). The organic layer was separated, dried over sodium sulfate, and filtered. The filtrate was concentrated to a residue and loaded directly onto a 40-g silica column (Teledyne Isco). The product was eluted from the silica using a 0-15% gradient of methanol in dichloromethane. The product was vacuum-dried at ambient temperature to afford the product, tofacitinib ethylene glycol monoglycinate as a tan solid [1.21 g, yield: 65%].

### Example 4

### Deprotection of Tofacitinib Ethylene Glycol Monoglycinate

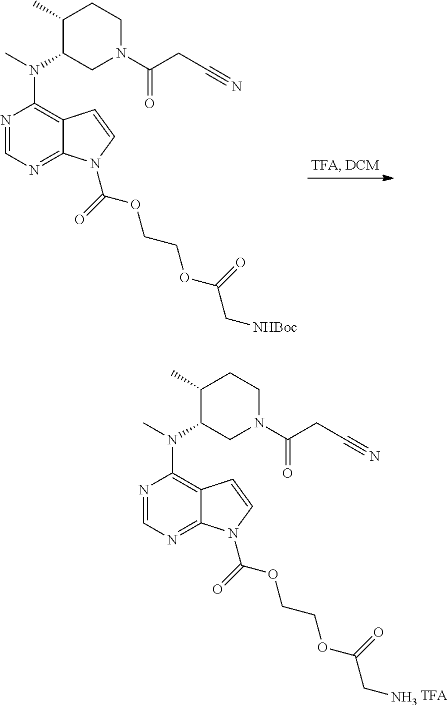 [SMILES: C[C@@H]1CCN(C(=O)CC#N)C[C@@H]1N(C)c1ncnc2c1ccn2C(=O)OCCOC(=O)CNC(=O)OC(C)(C)C ; C[C@@H]1CCN(C(=O)CC#N)C[C@@H]1N(C)c1ncnc2c1ccn2C(=O)OCCOC(=O)CN]

Tofacitinib ethylene glycol monoglycinate (300 mg, 0.54 mmol) was dissolved in a 1:1 mixture of dichloromethane and trifluoroacetic acid. The reaction mixture was stirred at ambient temperature for 30 minutes and was then evaporated under reduced pressure. The residue was triturated with three 5 mL portions of dichloromethane. The residue was then dissolved in methanol (1 mL) and precipitated into rapidly stirring diethyl ether (75 mL). The ether solution was stirred for 30 minutes and was then decanted. The residual gummy solid was dissolved in methanol and transferred to a tared 20 mL reaction vial. The product was vacuum-dried at ambient temperature to afford the product as a tan solid [280 mg, yield: 93%].

### Example 5

### Synthesis and Formulation of CDP-Tofacitinib Ethylene Glycol Monoglycinate Nanoparticles (CDP-Glycine-Tofacitinib Conjugate)

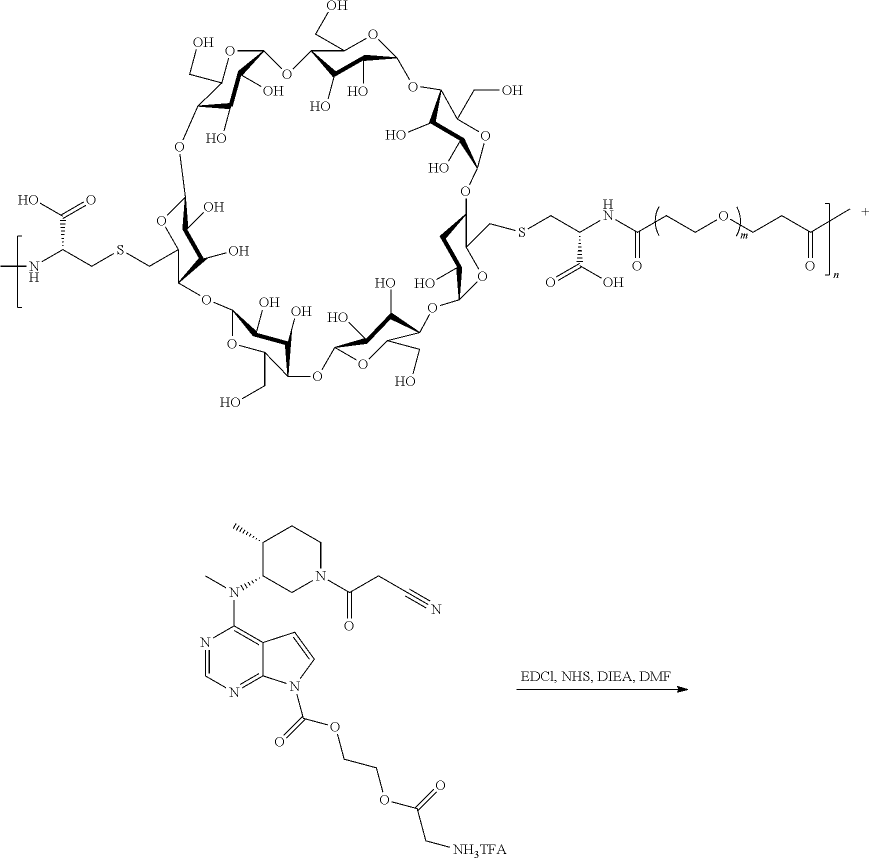

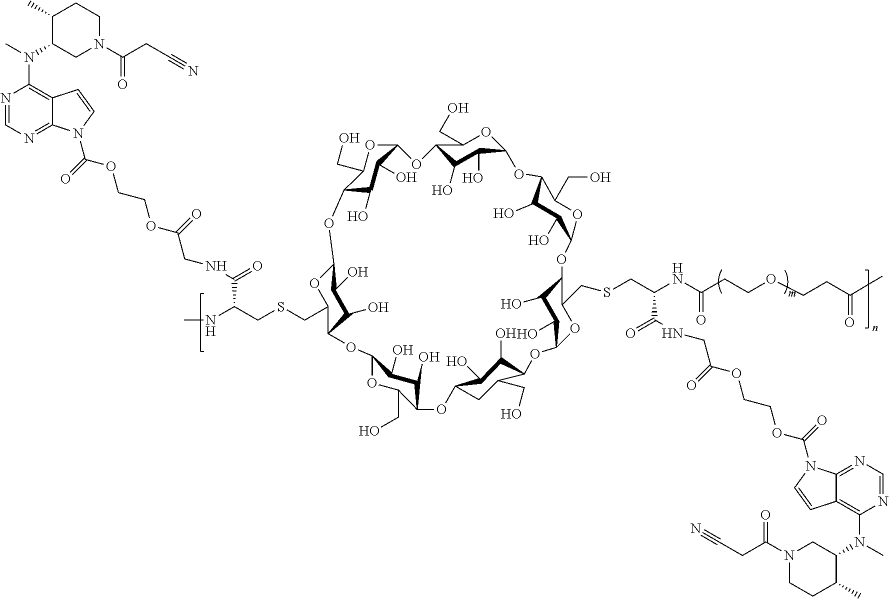

CDP (697 mg, 0.14 mmol) and deprotected tofacitinib ethylene glycol monoglycinate (270 mg, 0.49 mmol) were dissolved in anhydrous dimethylformamide (7 mL) and stirred for 30 minutes to dissolve the polymer. N-hydroxysuccinimide (NHS, 37 mg, 0.32 mmol), and 1-ethyl-3-(3-dimethylaminopropyl)carbodiimide (EDCI, 61 mg, 0.32 mmol) were added to the polymer solution. While stirring, N,N-diisopropylethylamine (DIEA, 164 mg, 1.27 mmol) was added and the stirring was continued for 5.5 hours.

The reaction mixture was worked up by precipitating the polymer in 15 volumes of acetone (150 mL). The polymer precipitated out immediately as a lump. The solution was stirred for 30 minutes and then the slightly turbid supernatant was decanted. The polymer precipitate was stirred in 10 additional volumes of acetone (200 mL) for 30 minutes and then dissolved 70 mL of water to prepare a ˜10 mg/mL polymer concentration. The polymer dissolved smoothly in water and the polymer solution was then filtered through a 0.22 μm PES membrane. This solution was then washed using TFF (3×30K capsules) using 10 volumes of ultrapure water at pH 3. After diafiltration, the solution was concentrated down to approximately half the volume and the concentrated solution was filtered with a 0.22 μm cellulose nitrate membrane. The filtered solution was analyzed for particle size using a particle sizer and Tofacitinib concentration using HPLC.

Particle properties, evaluated by using the resulting plurality of particles made in the method above:
Zavg=26.23 nm
Particle PDI=0.418
Dv50=11.4 nm
Dv90=19.8 nm

### Example 6

### Synthesis of Ethylene Glycol Monohexanoate

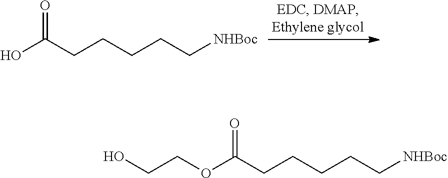 [SMILES: CC(C)(C)OC(=O)NCCCCCC(=O)O ; CC(C)(C)OC(=O)NCCCCCC(=O)OCCO]

A 300 mL round-bottom flask equipped with a magnetic stirrer was charged with N-Boc-6-amino hexanoic acid (2.5 g, 10.8 mmol), ethylene glycol (3.355 g, 54 mmol), N,N-dimethylaminopyridine (185 mg, 1.5 mmol) and dichloromethane (100 mL). The mixture was cooled in an ice bath and stirred for 5 minutes to produce a clear solution, to which 1-ethyl-3-(3-dimethyllaminopropyl)carbodiimide hydrochloride (EDC.HCl) (2.684 g, 14.1 mmol) was added. The mixture was stirred at ambient temperature for 18 hours and then the reaction mixture was diluted with DCM (200 mL) and washed with 1N HCl (150 mL), distilled water (150 mL) and brine (150 mL). The organic layer was separated, dried over sodium sulfate, and filtered. The filtrate was concentrated to a residue and loaded directly onto a 40-g silica column (Teledyne Isco). The product was eluted from the silica using a 0-100% gradient of ethyl acetate in hexane. The product was vacuum-dried at ambient temperature to afford the product, ethylene glycol monohexanoate as a clear oil [2.6 g, yield: 87%]. The ¹H NMR analysis was consistent with the assigned structure of the desired product.

### Example 7

### Synthesis of CDI Activated Ethylene Glycol Monohexanoate

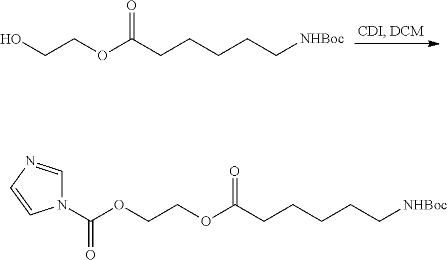 [SMILES: CC(C)(C)OC(=O)NCCCCCC(=O)OCCO ; CC(C)(C)OC(=O)NCCCCCC(=O)OCCOC(=O)n1ccnc1]

A 100 mL round-bottom flask equipped with a magnetic stir bar was charged with ethylene glycol monohexanoate (1.292 g, 4.69 mmol) and dichloromethane (10 mL). The mixture was stirred for 5 min to produce a clear solution, to which carbonyl diimidazole (1.522 g, 9.38 mmol). The reaction stirred at ambient temperature for 18 h and then diluted with DCM (100 mL). The organic layer was washed with distilled water (2×100 mL) and brine (1×100 mL) and then concentrated under reduced pressure to afford the product as a white solid [1.32 g, yield: 76%]. The ¹H NMR analysis was consistent with the assigned structure of the desired product.

### Example 8

### Synthesis of Tofacitinib Ethylene Glycol Monohexanoate

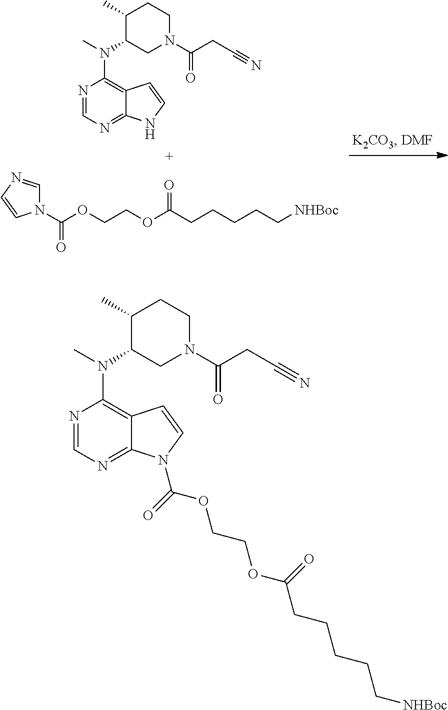 [SMILES: C[C@@H]1CCN(C(=O)CC#N)C[C@@H]1N(C)c1ncnc2[nH]ccc12 ; CC(C)(C)OC(=O)NCCCCCC(=O)OCCOC(=O)n1ccnc1 ; C[C@@H]1CCN(C(=O)CC#N)C[C@@H]1N(C)c1ncnc2c1ccn2C(=O)OCCOC(=O)CCCCCNC(=O)OC(C)(C)C]

A 20 mL reaction vial equipped with a magnetic stir bar was charged with Tofacitinib (827 mg, 2.65 mmol), and carbonyl diimidazole activated ethylene glycol monoglycinate (977 mg, 2.65 mmol), and anhydrous dimethylformamide (10 mL). The reaction mixture was stirred for 5 minutes to produce a clear solution, to which anhydrous potassium carbonate (1.095 g, 7.95 mmol) was added. The reaction mixture was stirred at ambient temperature overnight and was then diluted with DCM (200 mL). The organic layer was washed with 1N HCl (1×00 mL), distilled water (2×100 mL), and brine (1×100 mL). The organic layer was separated, dried over sodium sulfate, and filtered. The filtrate was concentrated to a residue and loaded directly onto a 40-g silica column (Teledyne Isco). The product was eluted from the silica using a 0-15% gradient of methanol in dichloromethane. The product was vacuum-dried at ambient temperature to afford the product, tofacitinib ethylene glycol monohexanoate as a tan solid [1.52 g, yield: 93%].

### Example 9

### Deprotection of Tofacitinib Ethylene Glycol Monohexanoate

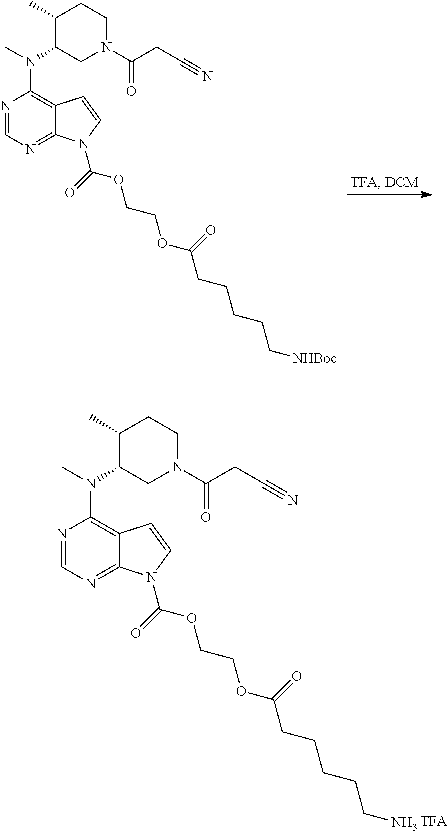 [SMILES: C[C@@H]1CCN(C(=O)CC#N)C[C@@H]1N(C)c1ncnc2c1ccn2C(=O)OCCOC(=O)CCCCCNC(=O)OC(C)(C)C ; C[C@@H]1CCN(C(=O)CC#N)C[C@@H]1N(C)c1ncnc2c1ccn2C(=O)OCCOC(=O)CCCCCN]

Tofacitinib ethylene glycol monohexanoate (170 mg, 0.28 mmol) was dissolved in a 1:1 mixture of dichloromethane and trifluoroacetic acid. The reaction mixture was stirred at ambient temperature for 30 minutes and was then evaporated under reduced pressure. The residue was triturated with three 5 mL portions of dichloromethane. The residual gummy solid was dissolved in methanol and transferred to a tared 20 mL reaction vial. The product was vacuum-dried at ambient temperature to afford the product as a tan solid [158 mg, yield: 92%].

### Example 10

### Synthesis and Formulation of CDP-Tofacitinib Ethylene Glycol Monohexanoate Nanoparticles (CDP-Hexanoate-Tofacitinib Conjugate)

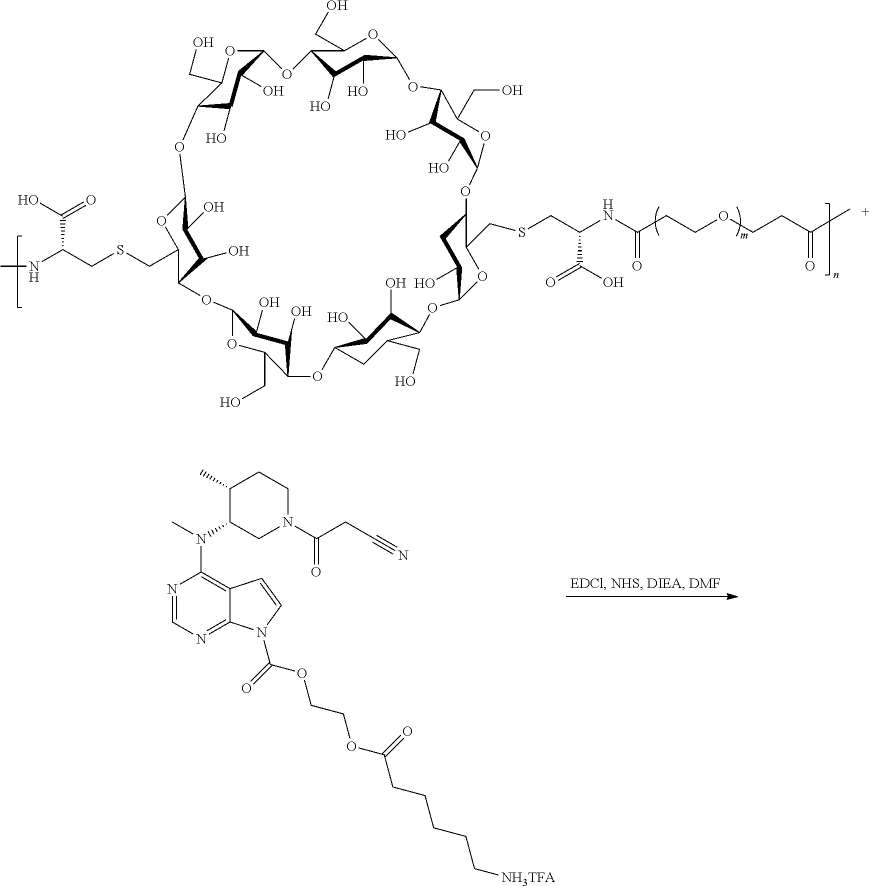

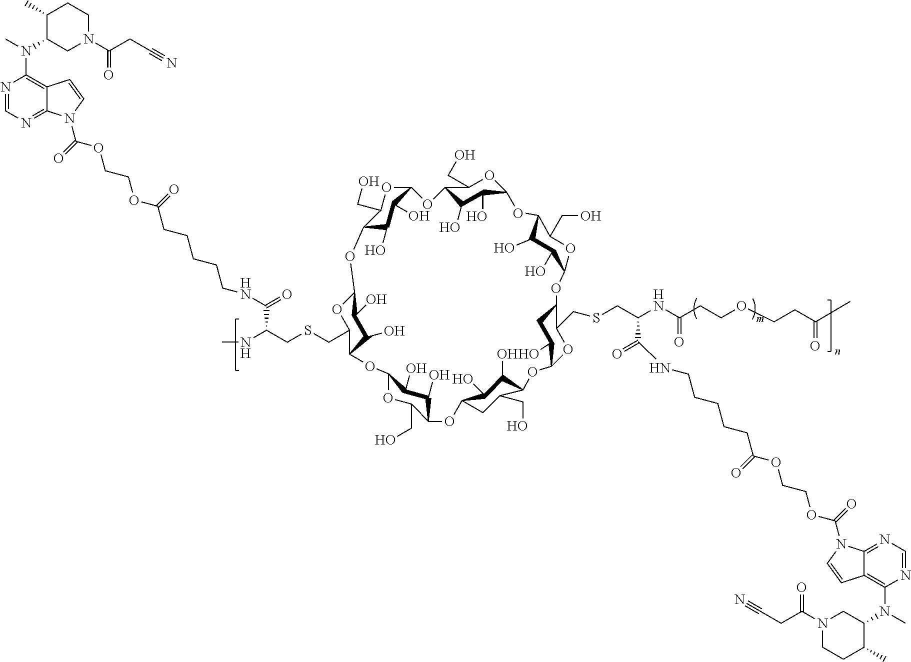

CDP (875 mg, 0.18 mmol) and deprotected tofacitinib ethylene glcol monohexanoate (360 mg, 0.59 mmol) were dissolved in anhydrous dimethylformamide (9 mL) and stirred for 30 minutes to dissolve the polymer. N-hydroxysuccinimide (NHS, 46 mg, 0.4 mmol), and 1-ethyl-3-(3-dimethylaminopropyl)carbodiimide (EDCI, 76 mg, 0.4 mmol) were added to the polymer solution. While stirring, N,N-diisopropylethylamine (DIEA, 206 mg, 1.6 mmol) was added and the stirring was continued for 5.5 hours.

The reaction mixture was worked up by precipitating the polymer in 15 volumes of acetone (150 mL). The polymer precipitated out immediately as a lump. The solution was stirred for 30 minutes and then the slightly turbid supernatant was decanted. The polymer precipitate was stirred in 10 additional volumes of acetone (200 mL) for 30 minutes and then dissolved in 90 mL of water to prepare a ˜10 mg/mL polymer concentration. The polymer dissolved smoothly in water and the polymer solution was then filtered through a 0.22 μm PES membrane. This solution was then washed using TFF (3×30K capsules) using 10 volumes of ultrapure water at pH 3. After diafiltration, the solution was concentrated down to approximately half the volume and the concentrated solution was filtered with a 0.22 μm cellulose nitrate membrane. The filtered solution was analyzed for particle size using a particle sizer and Tofacitinib concentration using HPLC.

Particle properties, evaluated by using the resulting plurality of particles made in the method above:
Zavg=20.64 nm
Particle PDI=0.218
Dv50=12.8 nm
Dv90=21.7 nm

### Example 11

### Drug Release and Stability Method for the CDP-Linker-Tofacitinib Conjugate Nanoparticles

The drug release and stability method experiments were run using the following CDP-linker-Tofacitinib nanoparticles: CDP-Gly-Tofac (prepared by the method described in Example 5) and CDP-Hex-Tofac) (prepared by the method described in Example 10).

A 2.5 mg/mL (with regard to polymer) solution of each CDP-linker-Tofac nanoparticle was prepared in 1×PBS buffer (pH=7.4). An aliquot of 150 μL was transferred into corresponding HPLC vials. A vial containing each CDP-linker-Tofac nanoparticle in PBS pH 7.4 for each designated time point was placed in a water bath at 37° C. Samples were mixed using a water bath shaker at 100 rpm during the experiments. At each designated time point, a vial was removed for each CDP-linker-Tofac nanoparticle and processed for HPLC using a sample preparation procedure.

To prepare a sample for HPLC analysis, each vial containing 150 μL of sample was mixed with 75 μL of 0.1% formic acid in acetonitrile. If there was any precipitated material in the vial, the contents were also stirred to dissolve the precipitate. If the sample was still opaque, an additional 25 μL of 0.1% formic acid in acetonitrile was added. HPLC analysis was used to determine the amount of free Tofacitinib and the amount of conjugated Tofacitinib in the sample for a given time point.

For the HPLC analysis at each time point, the peak areas of all relevant peaks from the chromatograms were retrieved and the concentration of free and conjugated Tofacitinib was calculated. The sample degradation was calculated based on the percentage of the amount of conjugated drug with regard to the initial starting point of the experiment (at t=0). The drug release was calculated based on the sum of free Tofacitinib and Tofacitinib main degradants at each time point. The drug release and degradation of given conjugate at 37° C. in 1×PBS after 24 h are presented in Table 1.

TABLE 1
Drug Release for CDP-Tofacitinib Conjugate Nanoparticles with
Glycine and Hexanoate Linker at 37° C. in 1x PBS at pH = 7.4

| | In vitro release | In vitro degradation |
|---|---|---|
| | of free drug | of conjugate |
| | (24 hrs in PBS | (24 hrs in PBS |
| Conjugate | at 37° C.) | at 37° C.) |
| CDP-Glycine-Tofacitinib | 9.45% | 15.23% |
| CDP-Hexanoate-Tofacitinib | 2.06% | 3.54% |

The data in Table 1 indicates that the hexanoate linker of the CDP-Hex-Tofac is relatively stable toward hydrolysis in vitro with 2.06% (free drug/conjugated drug) of free Tofacitinib detected after 24 hours. The glycine linker of the CDP-Glycine-Tofac was more susceptible to hydrolysis in vitro with 9.45% (free drug/conjugated drug) of free Tofacitinib detected after 24 hours. The data indicates that the relative stability of the CDP-Hexanoate-Tofacitinib conjugate was greater than the relative stability of the CDP-Glycine-Tofacitinib conjugate.

### Example 12

### Pharmacokinetic Studies for CDP-Tofacitinib Conjugate Nanoparticles with Glycine and Hexanoate Linkers

### Summary

Tofacitinib is a recently approved drug for rheumatoid arthritis which is an active JAK inhibitor. Tofacitinib is dosed 5 mg PO (orally) twice daily, and has a half-life of 3-5 hours. The goal of this work was to design nanoparticles which could be dosed subcutaneously to improve overall systemic exposure and reduce dose frequency. Two conjugates were synthesized and coupled to CDP with the intent to use the properties inherent in the nanoparticles formed to enable extended exposure via intravenous and subcutaneous administration. Two different linkers were tested, the hexanoate (CDP-Hexanoate-Tofacitinib Conjugate), and the glycine-based linker (CDP-Glycine-Tofacitinib Conjugate). Based on the PK data, CDP conjugates show substantial improvements over the approved PO route of administration for the unconjugated tofacitinib parent drug.

### Pharmacokinetics

Mice (C57 B16 strain) were treated by single bolus injection of each linker at 3 mg/kg. Blood samples were collected from individual animals and plasma made using EDTA as an anticoagulant. Levels of free and bound Tofacitinib were measured in the plasma samples using HPLC/MS/MS.

CDP-Hexanoate-Tofacitinib Conjugate Nanoparticles:

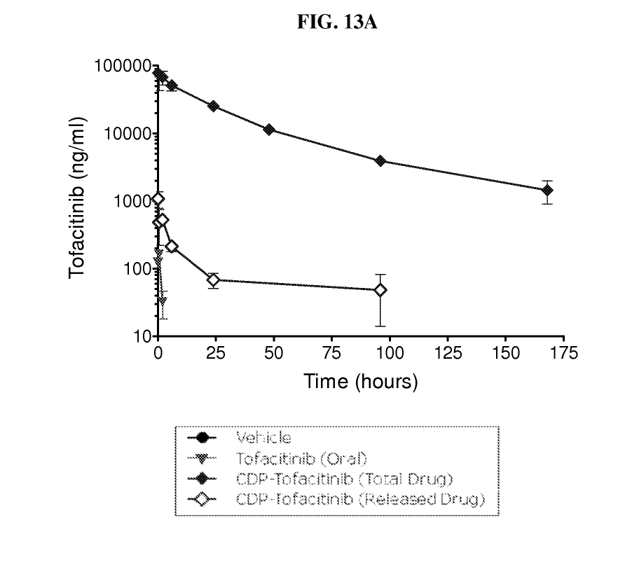

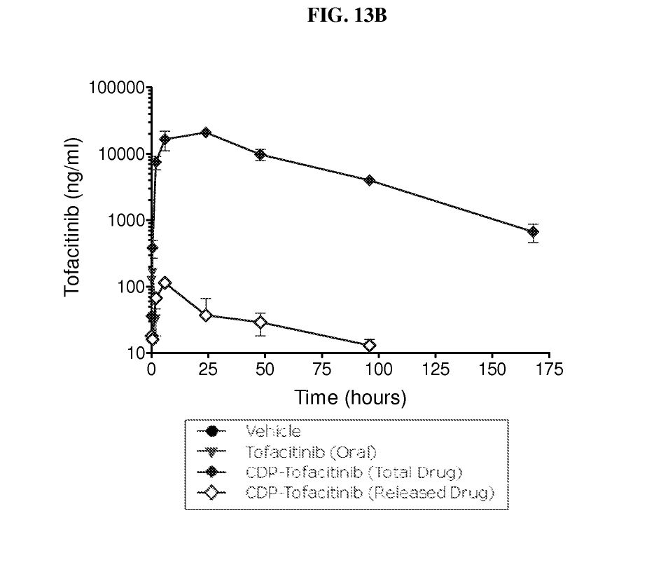

Concentration-time curves and PK parameters for the CDP-Hexanoate-Tofacitinib Conjugate are shown in FIG. 13A and FIG. 13B. Both the intravenous (IV) (FIG. 13A) and subcutaneous (SC) (FIG. 13B) routes showed substantial improvement in systemic exposure relative to the parent drug dosed orally. The CDP-hexanoate-tofacitinib conjugate nanoparticles showed low clearance, increased area under curve (AUC), and long half-life (Table 2). Based on the low volume of distribution for the conjugate, it was apparent that the nanoparticle did not have extensive tissue distribution. Levels of released drug were approximately 5% of the total and exhibited similar behaviors. SC dosing arms were included in this study to assess the feasibility of this route as it may be more patient-friendly. The half-life for conjugate increased by two fold relative to the IV dose, and the bioavailability was approximately 60% of the IV based on AUC.

TABLE 2
Plasma half-life and AUC for CDP-Hexanoate-Tofacitinib
Conjugate vs. the parent tofacitinib drug

| | | AUC, | | | |
|---|---|---|---|---|---|
| | Route | ng*hr/mL | Cl, ng/mL | V_{d,} mL | t_{1/2,} hr |
| Conjugate | IV | 1030865 | 0.07 | 2.4 | 23.2 |
| Free Drug | IV | 52472 | 1.4 | 45 | 22 |
| Conjugate | SC | 569866 | 0.132 | 8.9 | 47 |
| Free Drug | SC | 33055 | 2.27 | 63.3 | 19.3 |

CDP-Glycine-Tofacitinib Conjugate Nanoparticle:

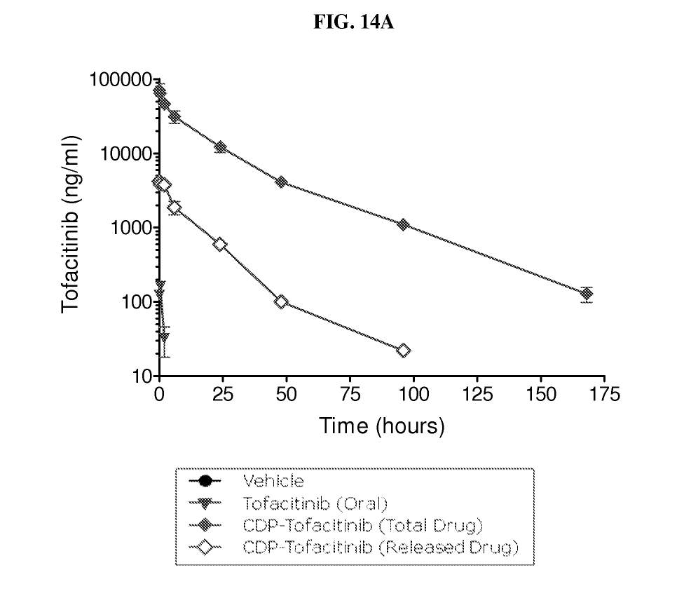

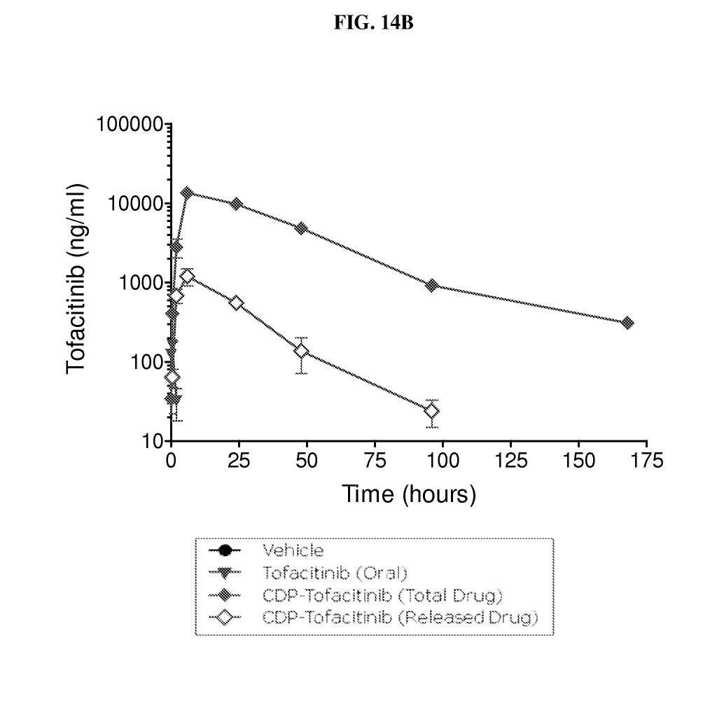

Concentration-time curves and PK parameters for the CDP-glycine-tofacitinib conjugate nanoparticles are shown in FIG. 14A and FIG. 14B. The curves were similar to the CDP-Hexanoate-tofacitinib conjugate nanoparticles in that they showed the same general behavior, but the glycine-linked material clearly circulated longer and released parent drug more slowly. Both the intravenous (IV) (FIG. 14A) and subcutaneous (SC) (FIG. 14B) routes showed substantial improvement in systemic exposure relative to the tofacitinib parent drug dosed orally. In this case, the CDP-glycine-tofacitinib conjugate showed free levels less than 1% of the circulating conjugate. The conjugate showed low clearance, increased area under curve (AUC), and long half-life (Table 3). The SC route for the CDP-glycine-tofacitinib conjugate nanoparticle showed the same lowered Cmaxand long half-life as with the hexanoate linker A lowered Cmax is correlated to a lower toxicity as concentrations of the drug can be kept below the minimally toxic concentration (MTC). Bioavailability of the CDP-glycine-tofacitinib conjugate nanoparticle via SC was similar at 60% of the total IV AUC and also showed the same doubling of half-life.

TABLE 3
Plasma half-life and AUC for CDP-Glycine-Tofacitinib
Conjugate nanoparticle vs. the parent tofacitinib drug

| | | AUC, | | | |
|---|---|---|---|---|---|
| | Route | ng*hr/mL | Cl, ng/mL | V_{d,} mL | t_{1/2,} hr |
| Conjugate | IV | 2061850 | 0.04 | 2.6 | 50 |
| Free Drug | IV | 9207 | 8.15 | 1661 | 141 |
| Conjugate | SC | 1254162 | 0.06 | 2.4 | 28 |
| Free Drug | SC | 3621 | 20.7 | 1224 | 41 |

Critical Features Exhibited with the CDP-Glycine-Tofacitinib and CDP-Hexanoate-Tofacitinib Conjugates:

Tofacitinib exposure was substantially greater when linked to CDP. Relative to the drug dosed PO alone; the IV AUC was on the order of 1000× greater. Both the CDP-Glycine-Tofacitinib and the CDP-Hexanoate-Tofacitinib conjugate nanoparticles showed good (60%) bioavailability and extended half-life. Different linkers to CDP exhibited control over drug release and thus systemic exposure, allowing the potential to stay within a therapeutic window. Because the SC route showed a blunted Cmax, this data could result in lowered toxicity as the free drug could be kept below the minimally toxic concentration (MTC).

### Example 13

### Comparison of CDP-Hexanoate-Tofacitinib Conjugate Nanoparticles and Unconjugated Tofacitinib Parent Drug in a Lewis Rat Adjuvant-Induced Arthritis (AIA) Model

### Animals and Materials

Animal and Facility:

75 female Lewis Rats at the age of 6 weeks were allowed to acclimate for 7 days in the animal facility (Cephrim Biosciences, Inc., Wellesley, Mass.). Animal housing, handling and procedures followed protocols and guidelines approved by the Institutional Animal Care and Use Committee (IACUC) of Cephrim Biosciences, Inc.

### Methods

In Vivo Adjuvant-Induced Arthritis (AIA) in Lewis Rat:

The adjuvant used for induction of arthritis was purchased from Chondrex Inc. (Redmond, Wash.), a suspension that contains 10 mg/mL of heat-killed mycobacteria in incomplete Freund's adjuvant. On the day of induction, the rat was first anesthetized and 0.05 mL of the Complete Freund's Adjuvant (CFA) was subcutaneously injected into the sub-plantar area of the rat footpad. The needle was inserted just under the skin of the footpad pointing toward the ankle to maximize the immune response to the CFA. The rat body weight, paw volumes and clinical assessments were monitored closely after the induction. Once the secondary swelling appeared around the opposite rat paws or ankles, the rats were assigned in one of 10 groups (N=5) according to the severity of arthritis (e.g. the paw volume) and the treatments started at that time (see Table 4).

Rat arthritis was evaluated for body weight, paw volumes and clinical score once every other day. Special care including placing water and food gel supplements in the cages were given for those rats once it became difficult to move around due to their hind limb arthritis. The severity of arthritis was scored by visual inspection. Three un-injected paws were scored on a scale of 0 to 4, where 0=normal; 1, only on few digits; 2, on the paw; 3, on the ankle and 4, all swelling below ankle. The maximum score was 12. In addition to daily scores, the maximum arthritis index (MAI) was calculated at the end of the study for each rat by adding the greatest score recorded for each paw. Foot swelling was also determined objectively by measuring paw volume with a plethysmograph for the secondary arthritis (un-injected foot) and thickness using a caliper for the primary arthritis (injected foot).

TABLE 4
Study Design

| | | Dosing | | |
|---|---|---|---|---|
| Group | N | (mg/kg) | Schedule | Route |
| Vehicle PBS | 5 | 0 | q7dx2 | subcutaneous |
| Dexamethasone | 5 | 1 | q2dx7 | subcutaneous |
| Tofacitinib | 5 | 10 | bidx14 | oral |
| (unconjugated parent drug) | | | | |
| CDP-hexanoate- | 5 | 3 | q7dx2 | subcutaneous |
| tofacitinib conjugate | | | | |
| CDP-hexanoate- | 5 | 1 | q7dx2 | subcutaneous |
| tofacitinib conjugate | | | | |
| CDP-hexanoate- | 5 | 0.3 | q7dx2 | subcutaneous |
| tofacitinib conjugate | | | | |

Treatment Preparation:

Dexamethasone stock was first dissolved in dimethyl sulfoxide at 1 mg/ml and kept in dark at room temperature. The corresponding dosing solution was prepared prior to each treatment by a 1:10 fold dilution with phosphate buffered saline (PBS). The dexamethasone dosing solution was given to the rats subcutaneously (SC) at 10 mL/kg. Tofacitinib (unconjugated parent drug) was added in 5% methylcellulose at 1 mg/mL and 0.25% of Tween-80. A homogeneous dosing suspension of tofacitinib was prepared using a mechanical homogenizer. The tofacitinib suspension was prepared daily and was given to rats by an oral gavage at 10 mL/kg. CDP-hexanoate-tofacitinib conjugate nanoparticles were kept in 4° C. until the end of the dosing period. They were given to their respective groups of rats by subcutaneous route at 10 mL/kg.

Results:

Ankle swelling was ameliorated with CDP-hexanoate-tofacitinib conjugate treatment to a level of healthy rats (i.e., 100% of initial paw volume), with a slower response time relative to the unconjugated parent drug. The spleen weight declined with dexamethasone and tofacitinib treatment, but was maintained by the CDP-hexanoate-tofacitinib conjugate nanoparticles (Table 5). The results show that the average spleen weight did not decline with CDP-hexanoate-tofacitinib conjugate treatment, whereas it did decline with dexamethasone and unconjugated parent drug treatment.

TABLE 5
Summary of Treatments on Rat Adjuvant-induced Arthritis (AIA)

| | | | % Max | % Max | |
|---|---|---|---|---|---|
| | | | Change in | Change in Paw | AVE Spleen |
| Group | Dose/Schedule/Route | MAI | body weight | Volume | Weight (g) |
| Vehicle/PBS | --- , q7dx2, sc | 46 | −17.8% (d18) | +53.1% (d18) | 0.593 |
| Dexamethasone | 1 mg/kg, q2dx7, sc | 29 | −13.5% (d18) | +26.5% (d13) | 0.253 |
| Tofacitinib | 10 mg/kg, bidx14, po | 30 | −9.0% (d13) | +30.7% (D14) | 0.392 |
| Tofacitinib/CDP | 3 mg/kg, q7dx2, sc | 39 | −9.3% (d13) | +42.5% (d16) | 0.606 |
| Tofacitinib/CDP | 1 mg/kg, q7dx2, sc | 46 | −14.5% (d18) | +45.2% (d16) | 0.642 |
| Tofacitinib/CDP | 0.3 mg/kg, q7dx2, sc | 41 | −12.9% (d16) | +48.0% (d16) | 0.643 |

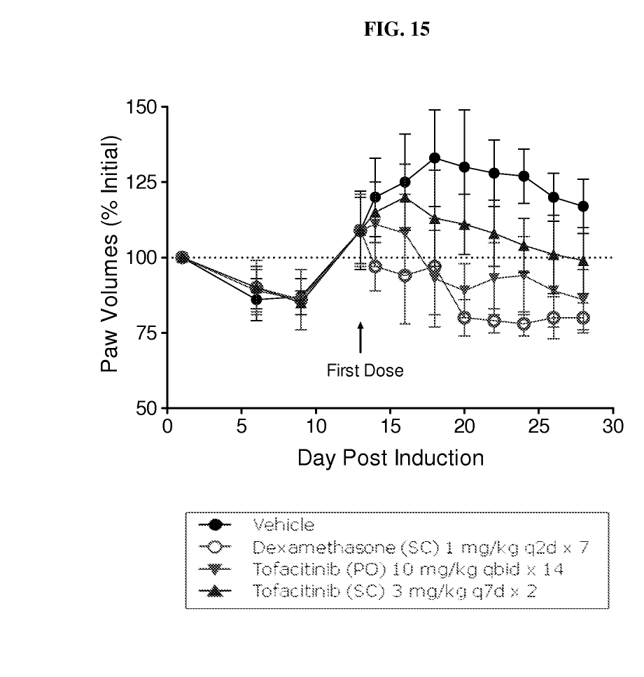

CDP-hexanoate-tofacitinib conjugate nanoparticles achieved similar anti-inflammatory activity as the unconjugated tofacitinib parent drug, despite being dosed at a cumulative dose of 3 mg/kg/week vs. 140 mg/kg/week for tofacitinib (Table 5). These results indicate that CDP-hexanoate-tofacitinib conjugate nanoparticles could achieve similar efficacy as unconjugated tofacitinib parent drug at 2% of the cumulative dose. (FIG. 15) Anti-inflammatory activity was achieved despite starting at a more inflamed state (due to slow release of drug from the nanoparticles). In addition, anti-inflammatory activity was achieved with subcutaneous dosing of CDP-hexanoate-tofacitinib conjugate nanoparticles, demonstrating that the nanoparticles could be dosed subcutaneously on a less frequent basis.

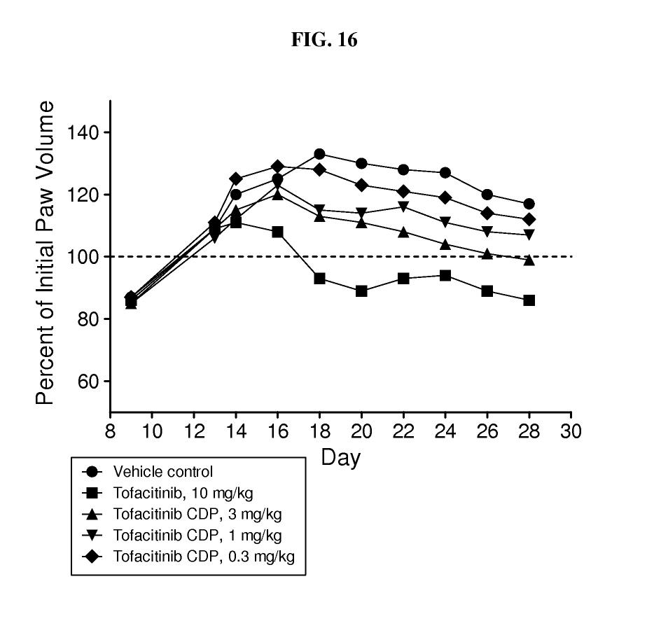

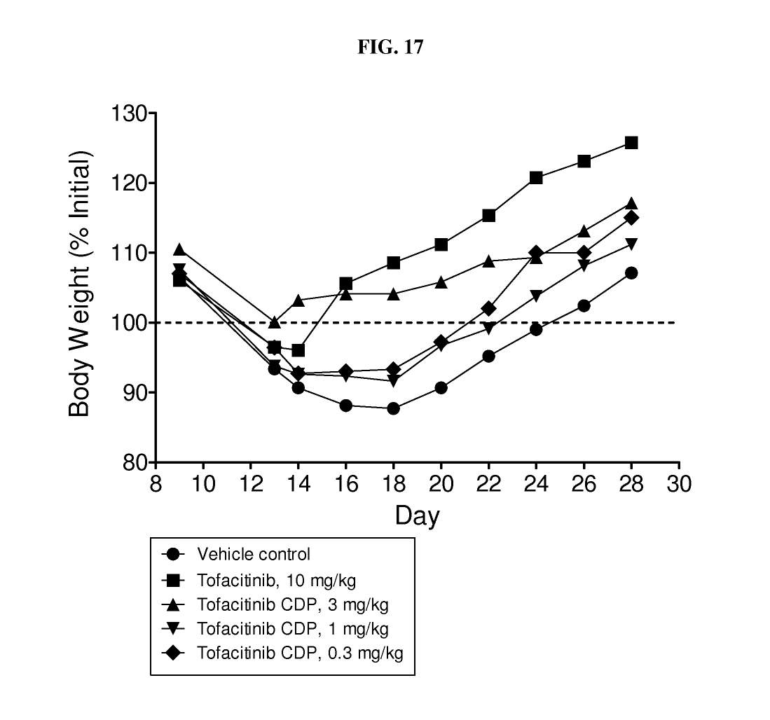

The effects of CDP-hexanoate-tofacitinib conjugate nanoparticles on rat paw volume as a percent of the initial paw volume at the time of arthritis induction (Day 1) is shown in FIG. 16, which indicated that paw volumes decreased to below 100% with CDP-hexanoate-tofacitinib conjugate nanoparticles dosed at 3 mg/kg q7d×2 after two cycles (28 days). The effects of CDP-hexanoate-tofacitinib conjugate nanoparticles on rat body weight in the AIA model as a percent of the initial body weight at the time of arthritis induction (Day 1) are shown in FIG. 17. The lack of weight loss observed in the group that received the highest dose (3 mg/kg q7d×2) was not related to the drug, since the dose was so low. Rather, the weight loss was related to the arthritic inflammation, and because the highest dose demonstrated the greatest anti-inflammatory acitivity, the weight loss was lower in that group.

Other embodiments are in the claims.

We claim:

1. A cyclodextrin-containing polymer-janus kinase (CDP-JAK) inhibitor conjugate.

2. The CDP-JAK inhibitor conjugate of claim 1, wherein the conjugate has the following formula: 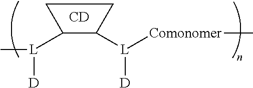
wherein
L is independently a linker;
each D is independently a janus kinase (JAK) inhibitor or absent, wherein the conjugate comprises at least one JAK inhibitor;
each comonomer comprises a hydrocarbylene group wherein one or more methylene groups of the hydrocarbylene group is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl;
and n is at least 4.

3. The CDP-JAK inhibitor conjugate of claim 2, wherein the comonomer comprises polyethylene glycol.

4. The CDP-JAK inhibitor conjugate of claim 2, wherein 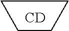
is alpha, beta or gamma cyclodextrin.

5. The CDP-JAK inhibitor conjugate of claim 2, wherein 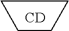
is beta cyclodextrin.

6. The CDP-JAK inhibitor conjugate of claim 2, wherein the linker comprises an alkylene chain, a polyethylene glycol (PEG) chain, polysuccinic anhydride, poly-L-glutamic acid, poly(ethyleneimine), an oligosaccharide, an amino acid, or an amino acid chain.

7. The CDP-JAK inhibitor conjugate of claim 2, wherein the linker comprises one or more amino acid.

8. The CDP-JAK inhibitor conjugate of claim 2, wherein the linker comprises cysteine or glycine or both.

9. The CDP-JAK inhibitor conjugate of claim 2, wherein the linker comprises glycine.

10. The CDP-JAK inhibitor conjugate of claim 2, wherein the linker comprises 6-aminohexanoic acid.

11. The CDP-JAK inhibitor conjugate of claim 2, wherein the linker comprises a self-cyclizing moiety and a selectivity-determining moiety.

12. The CDP-JAK inhibitor conjugate of claim 1, wherein the JAK inhibitor is conjugated to the cyclodextrin-containing polymer (CDP) through the self-cyclizing moiety of the linker.

13. The CDP-JAK inhibitor conjugate of claim 11, wherein the self-cyclizing moiety is covalently attached to the JAK inhibitor and comprises a structure: 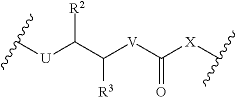
wherein
U is selected from O, NR¹ and S;
X represents a portion of the JAK inhibitor;
V is selected from O, S and NR⁴, preferably O or NR⁴;
R² and R³ are independently selected from hydrogen, alkyl, and alkoxy; or R² and R³ together with the carbon atoms to which they are attached form a ring; and
R¹, R⁴, and R⁵ are independently selected from hydrogen and alkyl.

14. The CDP-JAK inhibitor conjugate of claim 13, wherein U and V are each independently oxygen or NR¹, wherein R¹ is independently selected from hydrogen and alkyl.

15. The CDP-JAK inhibitor conjugate of claim 13, wherein U and V are each oxygen.

16. The CDP-JAK inhibitor conjugate of claim 13, wherein the self-cyclizing moiety covalently attached to the JAK inhibitor is selected from 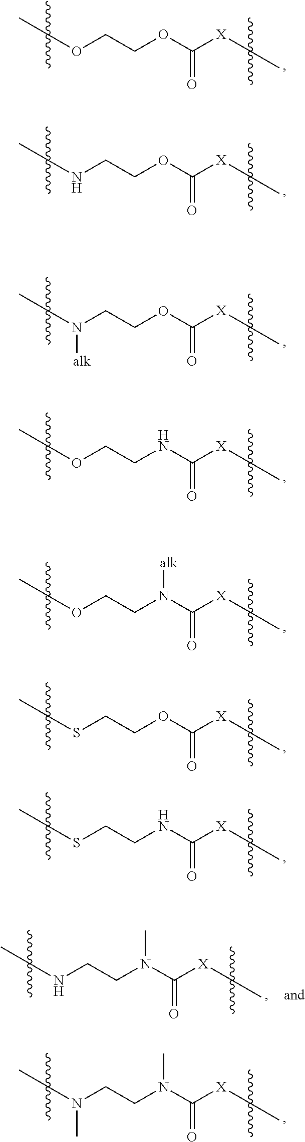
wherein “alk” is a C₁₋₆ alkyl group.

17. The CDP-JAK inhibitor conjugate of claim 16, wherein the self-cyclizing moiety attached to the JAK inhibitor is

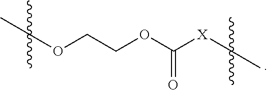

18. The CDP-JAK inhibitor conjugate of claim 12, wherein the self-cyclizing moiety is covalently attached to the JAK inhibitor and comprises a structure: 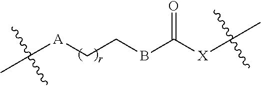
wherein A and B are heteroatoms independently selected from O, N, or S; X represents a portion of the JAK inhibitor; and r is 1, 2, or 3.

19. The CDP-JAK inhibitor conjugate of claim 12, wherein the selectivity-determining moiety comprises a structure: 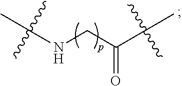
wherein p is 1 to 6.

20. The CDP-JAK inhibitor conjugate of claim 2, wherein the linker comprises a structure: 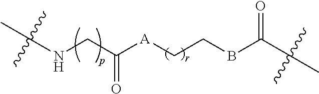
wherein
A and B are heteroatoms independently selected from O, N, or S;
p is 1 to 6; and
r is 1, 2, or 3.

21. The CDP-JAK inhibitor conjugate of claim 1, wherein the conjugate has the following formula: 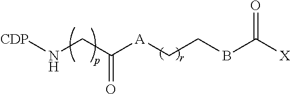
wherein
CDP is a cyclodextrin-containing polymer;
A and B are heteroatoms independently selected from O, N, or S;
X represents the JAK inhibitor;
p is 1 to 6; and
r is 1, 2, or 3.

22. The CDP-JAK inhibitor conjugate of claim 20 or 21, wherein at least one of A and B is oxygen.

23. The CDP-JAK inhibitor conjugate of claim 20 or 21, wherein A and B are each oxygen.

24. The CDP-JAK inhibitor conjugate of claim 1, wherein the JAK inhibitor is a JAK1 inhibitor, a JAK2 inhibitor, a JAK3 inhibitor, or a Tyk2 inhibitor.

25. The CDP-JAK inhibitor conjugate of claim 1, wherein the JAK inhibitor is selected from the group consisting of ruxolitinib, baricitinib, tofacitinib, GLPG0634, GSK2586184, VX-509, lestaurtinib, INCB16562, XL019, pacritinib, CYT387, AZD1480, TG101348, NVP-BSK805, CEP33779, R-348, AC-430, CDP-R723 and BMS 911543.

26. The CDP-JAK inhibitor conjugate of claim 1, wherein the JAK inhibitor is ruxolitinib, baricitinib, or tofacitinib.

27. The CDP-JAK inhibitor conjugate of claim 1, wherein the JAK inhibitor is tofacitinib.

28. A method of treating a disorder in a subject, the method comprising, administering a composition that comprises a cyclodextrin-containing polymer-janus kinase (CDP-JAK) inhibitor conjugate to a subject in an amount effective to treat the subject.

29. The method of claim 19, wherein the disorder is an inflammatory disorder, an autoimmune disorder, or a proliferative disorder.

30. The method of claim 29, wherein the proliferative disorder is a cancer.

31. The method of claim 28, wherein the CDP-JAK inhibitor conjugate is selected from the group consisting of a CDP-JAK1 conjugate, CDP-JAK2 conjugate, CDP-JAK3 conjugate, and a CDP-Tyk2 conjugate.

32. The method of claim 28, wherein the CDP-JAK inhibitor conjugate is selected from the group consisting of a CDP-ruxolitinib conjugate, a CDP-baricitinib conjugate, a CDP-tofacitinib conjugate, a CDP-GLPG0634 conjugate, a CDP-GSK2586184 conjugate, a CDP-VX-509 conjugate, a CDP-lestaurtinib conjugate, a CDP-INCB16562 conjugate, a CDP-XL019 conjugate, a CDP-pacritinib conjugate, a CDP-CYT387 conjugate, a CDP-AZD1480 conjugate, a CDP-TG101348 conjugate, a CDP-NVP-BSK805 conjugate, a CDP-CEP33779 conjugate a CDP-R-348 conjugate, a CDP-AC-430 conjugate, a CDP-R723 conjugate, and a CDP-BMS 911543 conjugate.

33. The method of claim 28, wherein the CDP-JAK inhibitor conjugate is a CDP-ruxolitinib conjugate, a CDP-baricitinib conjugate, or a CDP-tofacitinib conjugate.

34. The method of claim 28, wherein the disorder is an autoimmune or an inflammatory disorder.

35. The method of claim 28, wherein the disorder is selected from the group consisting of arthritis, diabetes, inflammatory bowel disease (IBD), and organ transplant rejection.

36. The method of claim 35, wherein the arthritis is crystalline arthritis, osteoarthritis, psoriatic arthritis, gouty arthritis, reactive arthritis, rheumatoid arthritis, or Reiter's arthritis.

37. The method of claim 35, wherein the diabetes is type I diabetes mellitus or type 2 diabetes mellitus.

38. The method of claim 35, wherein the IBD is Crohn's disease, ulcerative colitis, collagenous colitis, lymphocytic colitis, ischaemic colitis, diversion colitis, Behcet's syndrome, or indeterminate colitis.

39. The method of claim 28, wherein the method comprises administering the CDP-JAK inhibitor conjugate in combination with another therapy.

40. The method of claim 28, wherein the method comprises administering the CDP-JAK inhibitor conjugate in combination with an anti-metabolite or an anti-folate.

41. The method of claim 28, wherein the method comprises administering the CDP-JAK inhibitor conjugate in combination with methotrexate.

42. The method of claim 28, wherein the CDP-JAK inhibitor conjugate is administered subcutaneously.

43. The method of claim 28, wherein the CDP-JAK inhibitor conjugate is administered intravenously.
